(19) United States
(12) Patent Application Publication
(10) Pub. No.: US 2026/0052829 A1
(43) Pub. Date: Feb. 19, 2026

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Le Wang, Beijing (CN); Qiang Wang, Beijing (CN); Ziyan Zhang, Beijing (CN); Gaoqi Zhang, Beijing (CN); Jing Yan, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 19/294,578

(22) Filed: Aug. 8, 2025

(30) Foreign Application Priority Data
Aug. 16, 2024 (CN) 202411129917.1

(51) Int. Cl. H10K 50/11; C07D 413/10; C09K 11/06; H10K 85/30; H10K 85/40; H10K 85/60; H10K 101/00

(52) U.S. Cl. CPC H10K 50/11; C07D 413/10; C09K 11/06; H10K 85/342; H10K 85/40; H10K 85/626; H10K 85/633; H10K 85/636; H10K 85/654; H10K 85/656; H10K 85/6572; H10K 85/6574; H10K 85/6576; C09K 2211/185; H10K 2101/90

(57) ABSTRACT

Provided are an organic electroluminescent material and device thereof. The organic electroluminescent material is a compound having a structure of Formula 1, which may be used as a host material in the organic electroluminescent device. These novel compounds can provide better device performance such as high power efficiency and a longer lifetime. Further provided are the organic electroluminescent device comprising the compound, a compound composition comprising the compound, and an electronic apparatus comprising the compound.

### CROSS-REFERENCE TO RELATED APPLICATION(S)

This application claims priority to Chinese Patent Application No. 202411129917.1 filed on Aug. 16, 2024, the disclosure of which is incorporated herein by reference in its entirety.

### TECHNICAL FIELD

The present disclosure relates to compounds for organic electronic devices such as organic light-emitting devices. More particularly, the present disclosure relates to a compound having a structure of Formula 1, an organic electroluminescent device comprising the compound, a compound composition comprising the compound, and an electronic apparatus comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation (VTE method). Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

US20200010460A1 has disclosed a compound having a general formula of

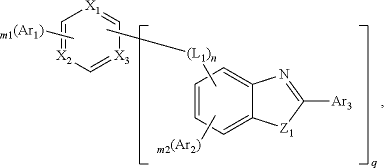

wherein the following structure is disclosed among specific structures of the compound:

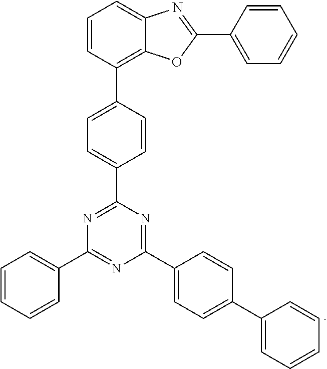 [SMILES: c1ccc(-c2ccc(-c3nc(-c4ccccc4)nc(-c4ccc(-c5cccc6nc(-c7ccccc7)oc56)cc4)n3)cc2)cc1]

This application focuses on the use of the compound as an electron transporting material to improve device performance and has disclosed or taught neither the use of the compound as a host material nor unique advantages due to the connection of a particular naphthylphenyl fragment to the triazinyl group of the compound.

US20230145469A1 has disclosed a general formula of

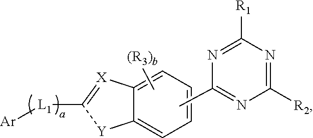

wherein a is an integer from 0 to 2, Ar is selected from hydrogen, deuterium, cyano, aryl, or heteroaryl, and R₁ and R₂ are each independently selected from aryl, fluorenyl, or a heterocyclic group. This application focuses on compounds formed by directly connecting benzoxazole and a triazinyl group by a single bond and has disclosed or taught neither compounds formed by connecting benzoxazole and a triazinyl group having a particular structure by a particular connection group nor that such compounds can improve device performance.

However, the currently reported host materials are still to be improved. To satisfy increasing requirements of the industry, especially requirements for performance such as higher device efficiency and a longer device lifetime, new materials still need to be further studied and developed.

### SUMMARY

The present disclosure aims to provide a series of compounds each having a structure of Formula 1 to solve at least part of the above-mentioned problems. These compounds may be used as host materials in organic electroluminescent devices. These novel compounds can provide better device performance.

According to an embodiment of the present disclosure, disclosed is a compound having a structure of Formula 1:

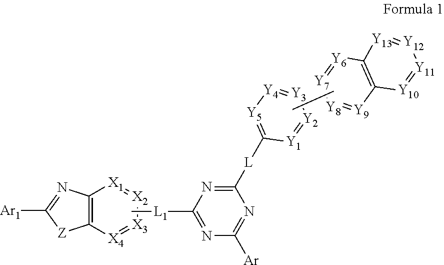

wherein
X₁ to X₄ are, at each occurrence identically or differently, selected from C, N, or CRₓ, and one of X₁ to X₄ is selected from C and joined to L₁;
Y₁ to Y₉ are, at each occurrence identically or differently, selected from C, N, or CR_{y}, one of Y₁ to Y₅ is selected from C, and one of Y₆ to Y₉ is selected from C;
Y₁₀ to Y₁₃ are, at each occurrence identically or differently, selected from N or CR_{y};
Z is, at each occurrence identically or differently, selected from O, S, or Se;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and when Rₓ is selected from substituted aryl having 6 to 30 carbon atoms, the aryl is substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar is selected from substituted or unsubstituted aryl having 6 to 30 carbon atoms, substituted or unsubstituted heteroaryl having 3 to 30 carbon atoms, or a combination thereof;
Ar₁ is selected from substituted or unsubstituted aryl having 6 to 30 carbon atoms;
L is selected from a single bond, substituted or unsubstituted arylene having 6 to 30 carbon atoms, substituted or unsubstituted heteroarylene having 3 to 30 carbon atoms, or a combination thereof;
L₁ is selected from substituted or unsubstituted arylene having 6 to 30 carbon atoms, substituted or unsubstituted heteroarylene having 3 to 30 carbon atoms, or a combination thereof;
adjacent substituents R_{y} in Y₁ to Y₅ can be optionally joined to form a ring; and
adjacent substituents R_{y} in Y₆ to Y₁₃ can be optionally joined to form a ring.

According to another embodiment of the present disclosure, further disclosed is an organic electroluminescent device comprising an anode, a cathode, and an organic layer disposed between the anode and the cathode, wherein the organic layer comprises a compound having a structure of Formula 1, and the compound has a specific structure described in the preceding embodiment.

According to another embodiment of the present disclosure, further disclosed is a compound composition comprising a compound having a structure of Formula 1, wherein the compound has a specific structure described in the preceding embodiment.

According to another embodiment of the present disclosure, further disclosed is an electronic apparatus comprising an organic electroluminescent device, wherein the organic electroluminescent device has a specific structure described in the preceding embodiment.

The novel compounds each having the structure of Formula 1 disclosed in the present disclosure may be used as the host materials in the electroluminescent devices. These novel compounds can provide better device performance such as higher power efficiency and a longer lifetime.

### BRIEF DESCRIPTION OF DRAWINGS

FIG. 1 is a schematic diagram of an organic light-emitting device that may comprise a compound and a compound composition disclosed herein.

FIG. 2 is a schematic diagram of another organic light-emitting device that may comprise a compound and a compound composition disclosed herein.

### DETAILED DESCRIPTION

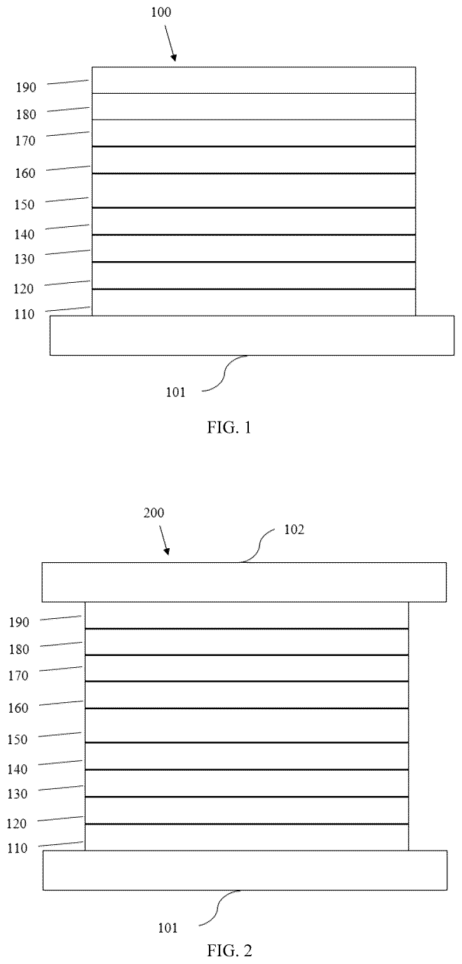

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbomyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl, and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

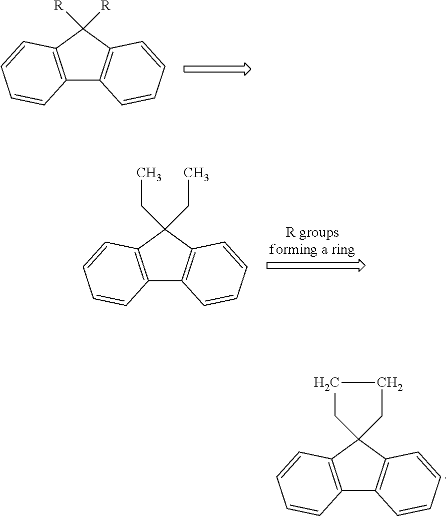

The expression that adjacent substituents can be optionally joined to form a ring is also intended to mean that two substituents bonded to carbon atoms which are directly bonded to each other are joined to each other via a chemical bond to form a ring, which can be exemplified by the following formula:

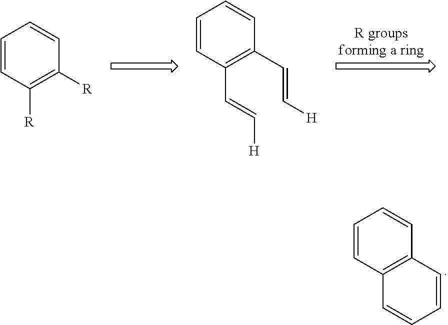

The expression that adjacent substituents can be optionally joined to form a ring is also intended to mean that two substituents bonded to further distant carbon atoms are joined to each other via a chemical bond to form a ring, which can be exemplified by the following formula:

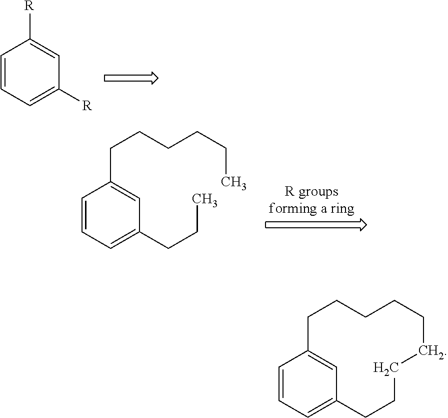

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

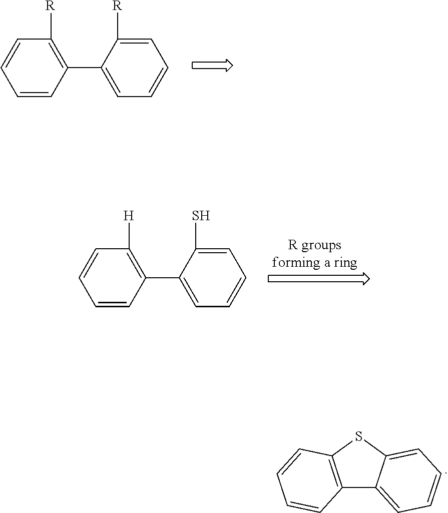

According to an embodiment of the present disclosure, disclosed is a compound having a structure of Formula 1:

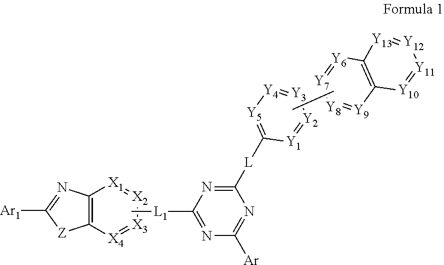

wherein
X₁ to X₄ are, at each occurrence identically or differently, selected from C, N, or CRₓ, and one of X₁ to X₄ is selected from C and joined to L₁;
Y₁ to Y₉ are, at each occurrence identically or differently, selected from C, N, or CR_{y}, one of Y₁ to Y₅ is selected from C, and one of Y₆ to Y₉ is selected from C;
Y₁₀ to Y₁₃ are, at each occurrence identically or differently, selected from N or CR_{y};
Z is, at each occurrence identically or differently, selected from O, S, or Se;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and when Rₓ is selected from substituted aryl having 6 to 30 carbon atoms, the aryl is substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar is selected from substituted or unsubstituted aryl having 6 to 30 carbon atoms, substituted or unsubstituted heteroaryl having 3 to 30 carbon atoms, or a combination thereof;
Ar₁ is selected from substituted or unsubstituted aryl having 6 to 30 carbon atoms;
L is selected from a single bond, substituted or unsubstituted arylene having 6 to 30 carbon atoms, substituted or unsubstituted heteroarylene having 3 to 30 carbon atoms, or a combination thereof;
L₁ is selected from substituted or unsubstituted arylene having 6 to 30 carbon atoms, substituted or unsubstituted heteroarylene having 3 to 30 carbon atoms, or a combination thereof;
adjacent substituents R_{y} in Y₁ to Y₅ can be optionally joined to form a ring; and
adjacent substituents R_{y} in Y₆ to Y₁₃ can be optionally joined to form a ring.

In the present disclosure, the expression that “adjacent substituents R_{y} in Y₁ to Y₅ can be optionally joined to form a ring” is intended to mean that any adjacent substituents R_{y} in Y₁ to Y₅ can be joined to form a ring. Obviously, it is also possible that any adjacent substituents R_{y} in Y₁ to Y₅ are not joined to form a ring.

In the present disclosure, the expression that “adjacent substituents R_{y} in Y₆ to Y₁₃ can be optionally joined to form a ring” is intended to mean that any adjacent substituents R_{y} in Y₆ to Y₁₃ can be joined to form a ring. Obviously, it is also possible that any adjacent substituents R_{y} in Y₆ to Y₁₃ are not joined to form a ring.

According to an embodiment of the present disclosure, none of substituents Rₓ in X₁ to X₄ can be joined to form a ring.

According to an embodiment of the present disclosure, the compound has a structure represented by any one of Formula 1-1 to Formula 1-4:

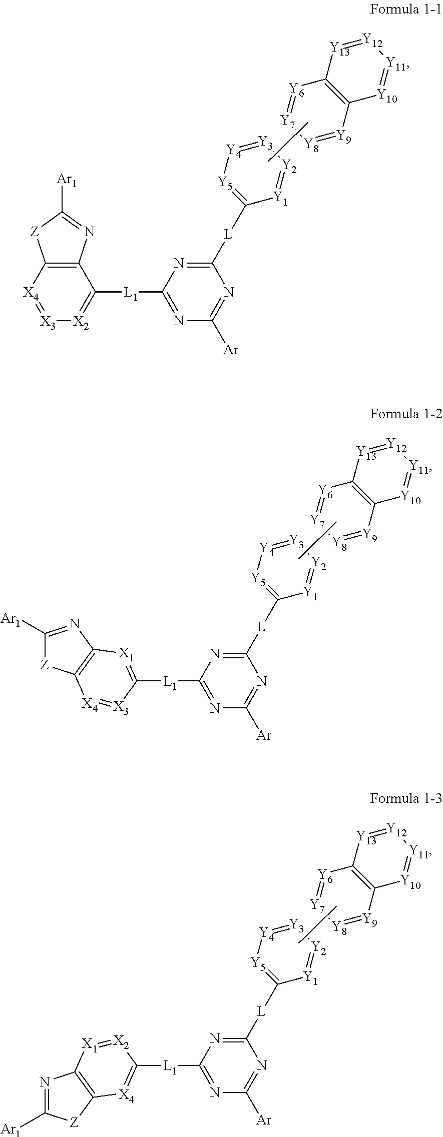

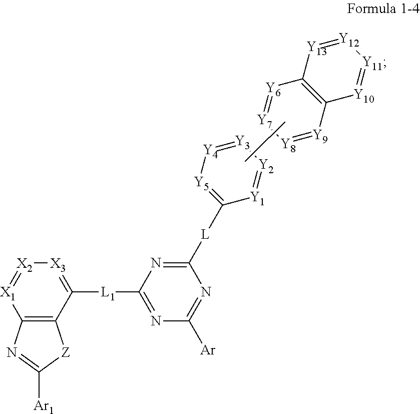

wherein
X₁ to X₄ are, at each occurrence identically or differently, selected from N or CRₓ;
Y₁ to Y₉ are, at each occurrence identically or differently, selected from C, N, or CR_{y}, one of Y₁ to Y₅ is selected from C, and one of Y₆ to Y₉ is selected from C;
Y₁₀ to Y₁₃ are, at each occurrence identically or differently, selected from N or CR_{y};
Z is, at each occurrence identically or differently, selected from O, S, or Se;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, a cyano group, an isocyano group, a hydroxyl group, a sulfanyl group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, a cyano group, an isocyano group, a hydroxyl group, a sulfanyl group, and combinations thereof; and when Rₓ is selected from substituted aryl having 6 to 30 carbon atoms, the aryl is substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar is selected from substituted or unsubstituted aryl having 6 to 30 carbon atoms, substituted or unsubstituted heteroaryl having 3 to 30 carbon atoms, or a combination thereof;
Ar₁ is selected from substituted or unsubstituted aryl having 6 to 30 carbon atoms;
L is selected from a single bond, substituted or unsubstituted arylene having 6 to 30 carbon atoms, substituted or unsubstituted heteroarylene having 3 to 30 carbon atoms, or a combination thereof;
L₁ is selected from substituted or unsubstituted arylene having 6 to 30 carbon atoms, substituted or unsubstituted heteroarylene having 3 to 30 carbon atoms, or a combination thereof;
adjacent substituents R_{y} in Y₁ to Y₅ can be optionally joined to form a ring; and
adjacent substituents R_{y} in Y₆ to Y₁₃ can be optionally joined to form a ring.

According to an embodiment of the present disclosure, R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, a cyano group, an isocyano group, a hydroxyl group, a sulfanyl group, and combinations thereof; and
Rₓ, is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, a cyano group, an isocyano group, a hydroxyl group, a sulfanyl group, and combinations thereof.

According to an embodiment of the present disclosure, R_{y} is, at each occurrence identically or differently, selected from the group consisting of: hydrogen, deuterium, fluorine, methyl, phenyl, deuterated naphthyl, deuterated phenyl, pyridyl, vinyl, naphthyl, biphenyl, phenanthryl, triphenylenyl, dibenzofuryl, dibenzothienyl, cyano, adamantyl, t-butyl, trifluoromethyl, carbazolyl, 9,9-dimethylfluorenyl, and combinations thereof; and
Rₓ, is, at each occurrence identically or differently, selected from the group consisting of: hydrogen, deuterium, fluorine, methyl, phenyl, deuterated naphthyl, deuterated phenyl, naphthyl, biphenyl, phenanthryl, triphenylenyl, cyano, adamantyl, t-butyl, trifluoromethyl, 9,9-dimethylfluorenyl, and combinations thereof.

According to an embodiment of the present disclosure, Z is selected from O or S.

According to an embodiment of the present disclosure, Z is selected from O.

According to an embodiment of the present disclosure, X₁ to X₄ are, at each occurrence identically or differently, selected from C or CRₓ, Y₁ to Y₉ are, at each occurrence identically or differently, selected from C or CR_{y}, and Y₁₀ to Y₁₃ are, at each occurrence identically or differently, selected from CR_{y}.

According to an embodiment of the present disclosure, Ar is selected from substituted or unsubstituted aryl having 6 to 18 carbon atoms, substituted or unsubstituted heteroaryl having 3 to 12 carbon atoms, or a combination thereof; and
Ar₁ is selected from substituted or unsubstituted aryl having 6 to 18 carbon atoms.

According to an embodiment of the present disclosure, Ar is selected from the group consisting of: substituted or unsubstituted naphthyl, substituted or unsubstituted phenyl, substituted or unsubstituted naphthylphenyl, substituted or unsubstituted terphenyl, substituted or unsubstituted biphenyl, substituted or unsubstituted phenanthryl, substituted or unsubstituted triphenylenyl, substituted or unsubstituted pyrimidinyl, substituted or unsubstituted pyridyl, substituted or unsubstituted dibenzofuryl, substituted or unsubstituted dibenzothienyl, substituted or unsubstituted 9,9-dimethylfluorenyl, and combinations thereof; and
Ar₁ is selected from the group consisting of: substituted or unsubstituted naphthyl, substituted or unsubstituted phenyl, substituted or unsubstituted naphthylphenyl, substituted or unsubstituted terphenyl, substituted or unsubstituted biphenyl, substituted or unsubstituted phenanthryl, substituted or unsubstituted triphenylenyl, substituted or unsubstituted 9,9-dimethylfluorenyl, and combinations thereof.

According to an embodiment of the present disclosure, L is selected from a single bond or substituted or unsubstituted arylene having 6 to 30 carbon atoms; and
L₁ is selected from substituted or unsubstituted arylene having 6 to 30 carbon atoms.

According to an embodiment of the present disclosure, L is selected from the group consisting of: a single bond, substituted or unsubstituted phenylene, substituted or unsubstituted biphenylylene, substituted or unsubstituted naphthylene, substituted or unsubstituted phenanthrylene, substituted or unsubstituted triphenylenylene, and combinations thereof; and
L₁ is selected from the group consisting of: substituted or unsubstituted phenylene, substituted or unsubstituted biphenylylene, substituted or unsubstituted naphthylene, substituted or unsubstituted phenanthrylene, substituted or unsubstituted triphenylenylene, and combinations thereof.

According to an embodiment of the present disclosure, L is selected from a single bond, and L₁ is selected from substituted or unsubstituted phenylene or substituted or unsubstituted naphthylene.

According to an embodiment of the present disclosure, one of Y₁ to Y₃ is selected from C and joined to Y₆ or Y₇.

According to an embodiment of the present disclosure, Y₃ is selected from C and joined to Y₆ or Y₇.

According to an embodiment of the present disclosure, the compound is selected from the group consisting of Compound 1-1 to Compound 1-230, wherein the specific structures of Compound 1-1 to Compound 1-230 are referred to claim 9.

According to an embodiment of the present disclosure, hydrogens in the structures of Compound 1-1 to Compound 1-230 can be partially or fully substituted with deuterium.

According to another embodiment of the present disclosure, further disclosed is an organic electroluminescent device comprising:
an anode,
a cathode, and
an organic layer disposed between the anode and the cathode, wherein the organic layer comprises a compound having a structure of Formula 1, and the compound has a specific structure described in any one of the preceding embodiments.

According to an embodiment of the present disclosure, in the organic electroluminescent device, the organic layer is an emissive layer, and the compound is a host material.

According to an embodiment of the present disclosure, in the organic electroluminescent device, the organic layer is an emissive layer and comprises a second compound, and the second compound is selected from a structure represented by any one of Formula 2-1 to Formula 2-3:

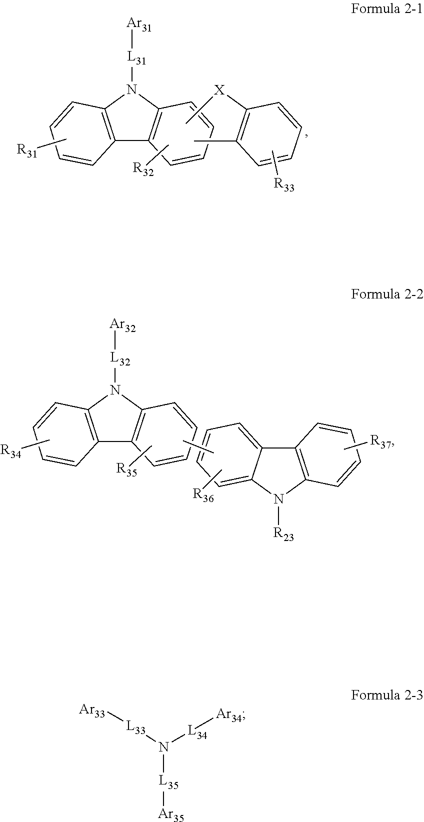

wherein
in Formula 2-1 to Formula 2-3, Ar₃₁ to Ar₃₅ are, at each occurrence identically or differently, selected from substituted or unsubstituted aryl having 6 to 30 carbon atoms or substituted or unsubstituted heteroaryl having 3 to 30 carbon atoms;
L₃₁ to L₃₅ are, at each occurrence identically or differently, selected from a single bond, substituted or unsubstituted arylene having 6 to 30 carbon atoms, substituted or unsubstituted heteroarylene having 3 to 30 carbon atoms, or a combination thereof;
X is, at each occurrence identically or differently, selected from O, S, CR₂₁R₂₂, or NR₂₃;
R₃₁ to R₃₇ represent, at each occurrence identically or differently, mono-substitution, multiple substitutions, or non-substitution;
R₃₁ to R₃₇ and R₂₁ to R₂₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₃₁ to R₃₇ can be optionally joined to form a ring.

In the present disclosure, the expression that “adjacent substituents R₃₁ to R₃₇ can be optionally joined to form a ring” is intended to mean that any one or more of groups of adjacent substituents, such as adjacent substituents R₃₁, adjacent substituents R₃₂, adjacent substituents R₃₃, adjacent substituents R₃₄, adjacent substituents R₃₅, adjacent substituents R₃₆, and adjacent substituents R₃₇, can be joined to form a ring. Obviously, it is also possible that none of these groups of adjacent substituents are joined to form a ring.

According to an embodiment of the present disclosure, Ar₃₁ to Ar₃₅ and R₂₁ to R₂₃ are, at each occurrence identically or differently, selected from the group consisting of: substituted or unsubstituted aryl having 6 to 18 carbon atoms, substituted or unsubstituted heteroaryl having 3 to 18 carbon atoms, and combinations thereof.

According to an embodiment of the present disclosure, Ar₃₁ to Ar₃₅ and R₂₁ to R₂₃ are, at each occurrence identically or differently, selected from the group consisting of: phenyl, deuterated phenyl, methylphenyl, fluorophenyl, t-butylphenyl, trideuteromethylphenyl, biphenyl, naphthyl, deuterated naphthyl, dibenzofuryl, dibenzothienyl, 9,9-dimethylfluorenyl, carbazolyl, pyridyl, pyrimidinyl, 4-cyanophenyl, 3-cyanophenyl, triphenylenyl, and combinations thereof.

According to an embodiment of the present disclosure, in the organic electroluminescent device, the organic layer is an emissive layer, and the second compound is a host material.

According to an embodiment of the present disclosure, in the organic electroluminescent device, the second compound has a structure represented by any one of Formula 2-4 to Formula 2-14:

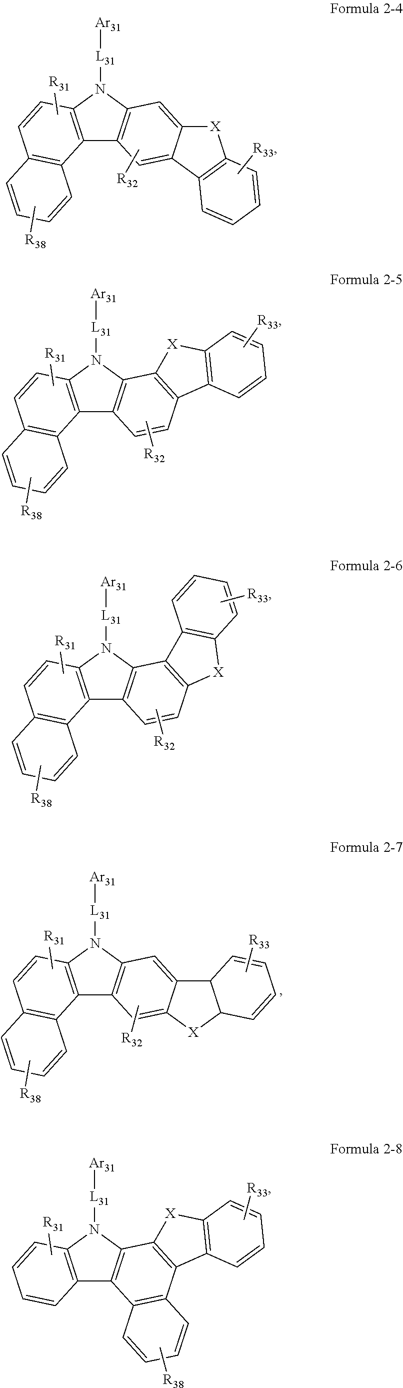

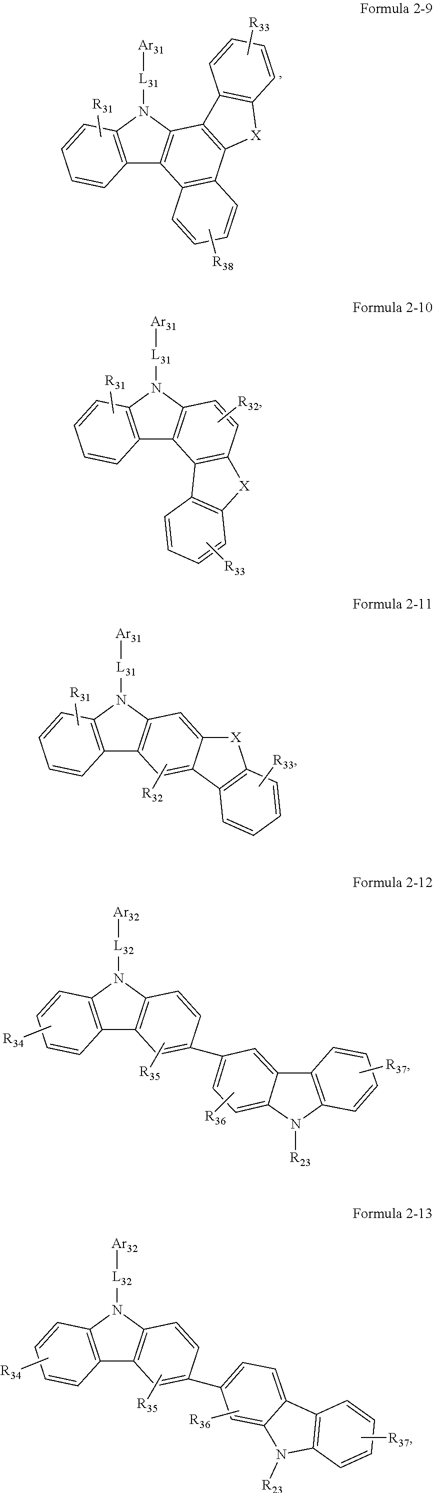

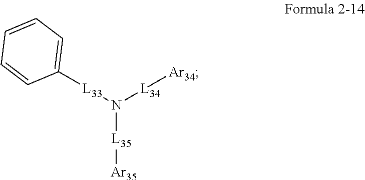

wherein in Formula 2-4 to Formula 2-14,
Ar₃₁, Ar₃₂, Ar₃₄, and Ar₃₅ are, at each occurrence identically or differently, selected from substituted or unsubstituted aryl having 6 to 30 carbon atoms or substituted or unsubstituted heteroaryl having 3 to 30 carbon atoms;
L₃₁ to L₃₅ are, at each occurrence identically or differently, selected from a single bond, substituted or unsubstituted arylene having 6 to 30 carbon atoms, substituted or unsubstituted heteroarylene having 3 to 30 carbon atoms, or a combination thereof;
X is, at each occurrence identically or differently, selected from O, S, CR₂₁R₂₂, or NR₂₃;
R₃₁ to R₃₈ represent, at each occurrence identically or differently, mono-substitution, multiple substitutions, or non-substitution;
R₃₁ to R₃₈ and R₂₁ to R₂₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₃₁ to R₃₈ can be optionally joined to form a ring.

In the present disclosure, the expression that “adjacent substituents R₃₁ to R₃₈ can be optionally joined to form a ring” is intended to mean that any one or more of groups of adjacent substituents, such as adjacent substituents R₃₁, adjacent substituents R₃₂, adjacent substituents R₃₃, adjacent substituents R₃₄, adjacent substituents R₃₅, adjacent substituents R₃₆, adjacent substituents R₃₇, and adjacent substituents R₃₈, can be joined to form a ring. Obviously, it is also possible that none of these groups of adjacent substituents are joined to form a ring.

According to an embodiment of the present disclosure, wherein the X is, at each occurrence identically or differently, selected from O, S, or NR₂₃.

According to an embodiment of the present disclosure, wherein the X is, at each occurrence identically or differently, selected from NR₂₃.

According to an embodiment of the present disclosure, wherein the R₃₁ to R₃₈ are, at each occurrence identically or differently, selected from the group consisting of: hydrogen, deuterium, halogen, cyano, hydroxyl, sulfanyl, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, wherein the R₃₁ to R₃₈ are, at each occurrence identically or differently, selected from the group consisting of: hydrogen, deuterium, fluorine, cyano, hydroxyl, sulfanyl, methyl, trideuteromethyl, vinyl, phenyl, biphenyl, naphthyl, 4-cyanophenyl, dibenzofuryl, dibenzothienyl, triphenylenyl, carbazolyl, 9-phenylcarbazolyl, 9,9-dimethylfluorenyl, pyridyl, phenylpyridyl, and combinations thereof.

According to an embodiment of the present disclosure, wherein the L₃₁ to L₃₅ are, at each occurrence identically or differently, selected from the group consisting of: a single bond, substituted or unsubstituted arylene having 6 to 18 carbon atoms, substituted or unsubstituted heteroarylene having 3 to 18 carbon atoms, and combinations thereof.

According to an embodiment of the present disclosure, wherein the L₃₁ to L₃₅ are, at each occurrence identically or differently, selected from the group consisting of: a single bond, phenylene, naphthylene, biphenylylene, terphenylene, triphenylenylene, pyridylene, thienylene, dibenzofurylene, dibenzothienylene, and combinations thereof.

According to an embodiment of the present disclosure, wherein the Ar₃₁ to Ar₃₅ and R₂₁ to R₂₃ are, at each occurrence identically or differently, selected from the group consisting of: substituted or unsubstituted aryl having 6 to 18 carbon atoms, substituted or unsubstituted heteroaryl having 3 to 18 carbon atoms, and combinations thereof.

According to an embodiment of the present disclosure, wherein the Ar₃₁ to Ar₃₅ and R₂₁ to R₂₃ are, at each occurrence identically or differently, selected from the group consisting of: phenyl, deuterated phenyl, methylphenyl, fluorophenyl, t-butylphenyl, trideuteromethylphenyl, biphenyl, naphthyl, deuterated naphthyl, dibenzofuryl, dibenzothienyl, 9,9-dimethylfluorenyl, carbazolyl, pyridyl, pyrimidinyl, 4-cyanophenyl, 3-cyanophenyl, triphenylenyl, and combinations thereof.

According to an embodiment of the present disclosure, wherein the second compound is selected from the group consisting of Compound H-1 to Compound H-179, wherein the specific structures of Compound H-1 to Compound H-179 are referred to claim 12.

According to an embodiment of the present disclosure, hydrogens in the structures of Compound H-1 to Compound H-179 can be partially or fully substituted with deuterium.

According to an embodiment of the present disclosure, during device preparation, when the compound of the present disclosure and the second compound together with an emissive material are to be co-evaporated to form an emissive layer, the compound of the present disclosure and the second compound may be co-evaporated from respective evaporation sources to form the emissive layer, or a premixed mixture of the compound of the present disclosure and the second compound may be loaded in a single evaporation source and evaporated to form the emissive layer.

According to an embodiment of the present disclosure, in the organic electroluminescent device, the organic layer is an emissive layer, and the emissive layer comprises at least one phosphorescent material.

According to an embodiment of the present disclosure, the phosphorescent material is a metal complex having a general formula of M(Lₐ)ₘ(L_{b})ₙ(L_{c})_{q};
M is selected from a metal with a relative atomic mass greater than 40;
Lₐ, L_{b}, and L_{c} are a first ligand, a second ligand, and a third ligand coordinated to M, respectively, and Lₐ, L_{b}, and L_{c} can be optionally joined to form a multidentate ligand;
Lₐ, L_{b}, and L_{c} are identical or different; m is 1, 2, or 3, n is 0, 1, or 2, q is 0, 1, or 2, and m+n+q is equal to the oxidation state of M; when m is greater than or equal to 2, multiple Lₐ are identical or different; when n is 2, two L_{b} are identical or different; when q is 2, two L_{c} are identical or different;
Lₐ is, at each occurrence identically or differently, selected from a structure represented by Formula 3:

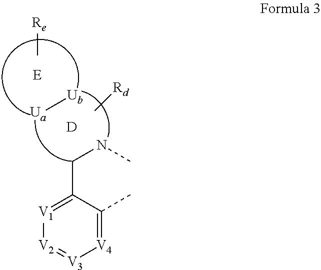

wherein
the ring D is selected from a five-membered heteroaromatic ring or a six-membered heteroaromatic ring;
the ring E is selected from a five-membered unsaturated carbocyclic ring, a benzene ring, a five-membered heteroaromatic ring, or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions, or non-substitution;
V₁ to V₄ are, at each occurrence identically or differently, selected from CRᵥ or N;
R_{d}, Rₑ, and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ, and Rᵥ can be optionally joined to form a ring;
L_{b} and L_{c} are, at each occurrence identically or differently, selected from any one of the following structures:

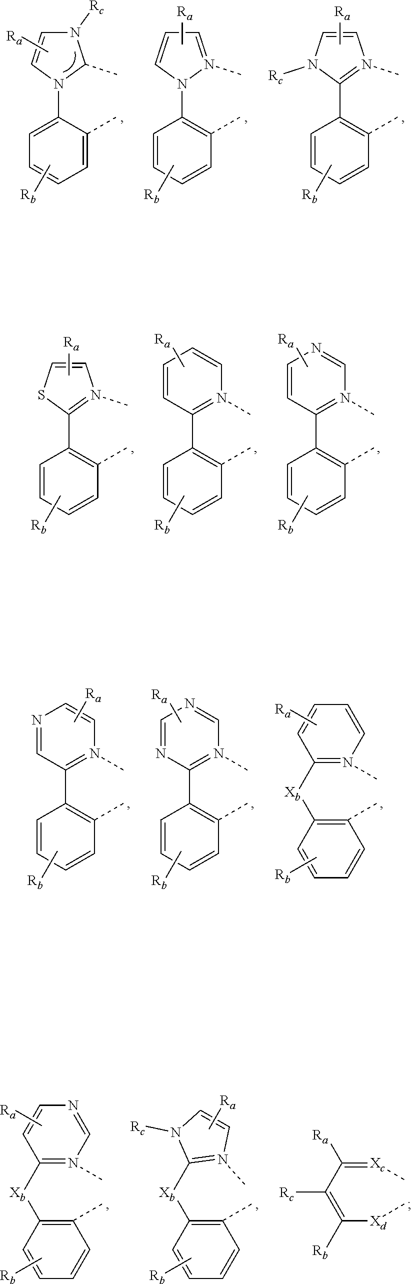

wherein
Rₐ, R_{b}, and R_{c} represent, at each occurrence identically or differently, mono-substitution, multiple substitutions, or non-substitution;
X_{b} is, at each occurrence identically or differently, selected from the group consisting of: O, S, Se, NR_{N1}, and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1}, and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
in the structures of the ligands L_{b} and L_{c}, adjacent substituents Rₐ, R_{b}, R_{c}, R_{N1}, R_{N2}, R_{C1}, and R_{C2} can be optionally joined to form a ring.

In the present disclosure, the expression that “adjacent substituents R_{d}, Rₑ, and Rᵥ can be optionally joined to form a ring” is intended to mean that when substituents R_{d}, Rₑ, and Rᵥ exist, any one or more of groups of adjacent substituents, such as adjacent substituents R_{d}, adjacent substituents Rₑ, adjacent substituents Rᵥ, adjacent substituents R_{d} and Rₑ, adjacent substituents R_{d} and Rᵥ, and adjacent substituents Rₑ and Rᵥ, can be joined to form a ring. Obviously, when substituents R_{d}, Rₑ, and Rᵥ exist, it is also possible that none of these groups of substituents are joined to form a ring.

In the present disclosure, the expression that “adjacent substituents Rₐ, R_{b}, R_{c}, R_{N1}, R_{N2}, R_{C1}, and R_{C2} can be optionally joined to form a ring” is intended to mean that any one or more of groups of adjacent substituents, such as two substituents Rₐ, two substituents R_{b}, two substituents R_{c}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents Rₐ and R_{N2}, substituents R_{b} and R_{N2}, and substituents R_{C1} and R_{C2}, can be joined to form a ring. For example, adjacent substituents Rₐ and Rₑ in

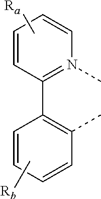

can be optionally joined to form a ring, which can form one or more of the following structures including, but not limited to,

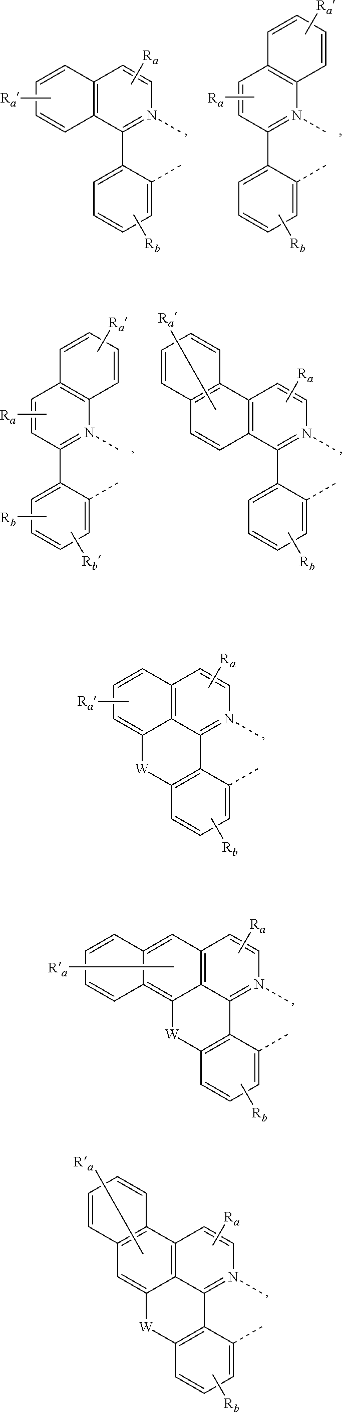

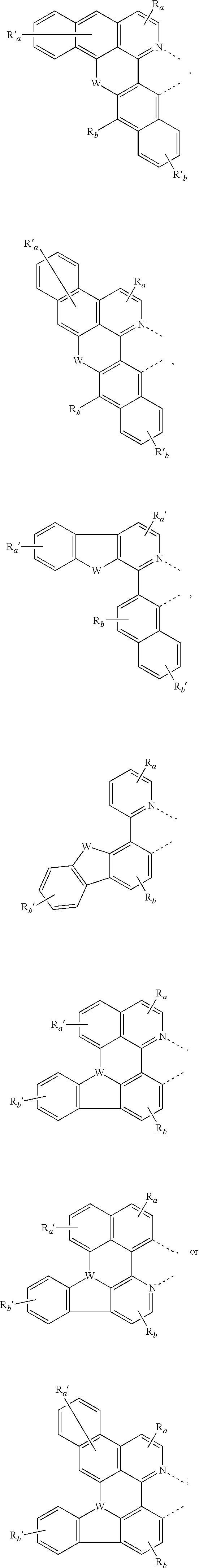

Wherein W is selected from O, S, Se, NR_{w}, or CR_{w}R_{w}, and R_{w}, Rₐ′, and R_{b}′ are defined the same as Rₐ. Obviously, it is also possible that none of these substituents are joined to form a ring.

According to an embodiment of the present disclosure, in Formula 3, two adjacent substituents Rₑ are joined to form a ring.

According to an embodiment of the present disclosure, in Formula 3, two adjacent substituents Rₑ are joined to form a five-membered unsaturated carbocyclic ring, a five-membered heteroaromatic ring, or a benzene ring.

According to an embodiment of the present disclosure, in Formula 3, the ring D is a six-membered heteroaromatic ring, and the ring E is a benzene ring or a six-membered heteroaromatic ring.

According to an embodiment of the present disclosure, in Formula 3, the ring D is a six-membered heteroaromatic ring, and the ring E is a five-membered heteroaromatic ring or a five-membered unsaturated carbocyclic ring.

According to an embodiment of the present disclosure, in Formula 3, the ring D is a six-membered heteroaromatic ring, the ring E is a benzene ring or a six-membered heteroaromatic ring, and two adjacent substituents Rₑ are joined to form a benzene ring or a six-membered heteroaromatic ring.

According to an embodiment of the present disclosure, in Formula 3, the ring D is a six-membered heteroaromatic ring, the ring E is a five-membered heteroaromatic ring or a five-membered unsaturated carbocyclic ring, and two adjacent substituents Rₑ are joined to form a benzene ring or a six-membered heteroaromatic ring.

According to an embodiment of the present disclosure, in Formula 3, at least one or two of groups of adjacent substituents R_{d}, Rₑ, and Rᵥ are joined to form a ring. For example, two substituents R_{d} are joined to form a ring, two substituents Rₑ are joined to form a ring, two substituents Rᵥ are joined to form a ring, substituents R_{d} and Rₑ are joined to form a ring, substituents R_{d} and Rᵥ are joined to form a ring, substituents Rₑ and Rᵥ are joined to form a ring, two substituents Rₑ are joined to form a ring while two substituents R_{d} are joined to form a ring, two substituents Rᵥ are joined to form a ring while two substituents R_{d} are joined to form a ring, two substituents Rᵥ are joined to form a ring while two substituents Rₑ are joined to form a ring, two substituents Rᵥ are joined to form a ring while substituents Rₑ and Rᵥ are joined to form a ring, or two substituents Rᵥ are joined to form a ring while substituents R_{d} and Rᵥ are joined to form a ring; more groups of adjacent substituents R_{d}, Rₑ, and Rᵥ are joined to form a ring similarly.

According to an embodiment of the present disclosure, in the organic electroluminescent device, the phosphorescent material is a metal complex having a general formula of M(Lₐ)ₘ(L_{b})ₙ;
M is selected from a metal with a relative atomic mass greater than 40;
Lₐ and L_{b} are a first ligand and a second ligand coordinated to M, respectively, and Lₐ and L_{b} can be optionally joined to form a multidentate ligand;
m is 1, 2, or 3, n is 0, 1, or 2, and m+n is equal to the oxidation state of M; when m is greater than or equal to 2, multiple Lₐ may be identical or different; when n is 2, two L_{b} may be identical or different;
Lₐ is, at each occurrence identically or differently, selected from a structure represented by Formula 3:

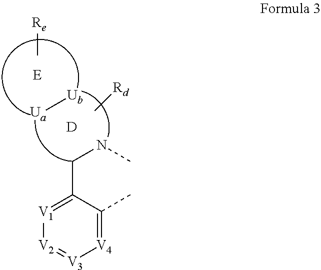

wherein
the ring D is selected from a five-membered heteroaromatic ring or a six-membered heteroaromatic ring;
the ring E is selected from a five-membered unsaturated carbocyclic ring, a benzene ring, a five-membered heteroaromatic ring, or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions, or non-substitution;
V₁ to V₄ are, at each occurrence identically or differently, selected from CRᵥ or N;
R_{d}, Rₑ, and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ, and Rᵥ can be optionally joined to form a ring;
the ligand L_{b} is, at each occurrence identically or differently, selected from the following structure:

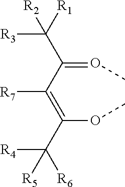

wherein R₁ to R₇ are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group, and combinations thereof.

According to an embodiment of the present disclosure, in the organic electroluminescent device, the ligand L_{b} is, at each occurrence identically or differently, selected from the following structure:

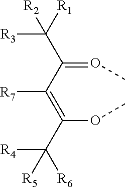

wherein at least one of R₁ to R₃ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof; and/or at least one of R₄ to R₆ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof.

According to an embodiment of the present disclosure, in the organic electroluminescent device, the ligand L_{b} is, at each occurrence identically or differently, selected from the following structure:

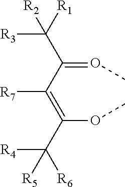

wherein at least two of R₁ to R₃ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof; and/or at least two of R₄ to R₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof.

According to an embodiment of the present disclosure, in the organic electroluminescent device, the ligand L_{b} is, at each occurrence identically or differently, selected from the following structure:

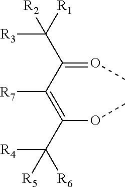

wherein at least two of R₁ to R₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms, or a combination thereof; and/or at least two of R₄ to R₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms, or a combination thereof.

According to an embodiment of the present disclosure, in the organic electroluminescent device, the phosphorescent material is an Ir complex, a Pt complex, or an Os complex.

According to an embodiment of the present disclosure, in the organic electroluminescent device, the phosphorescent material is an Ir complex and has a structure represented by any one of Ir(Lₐ)(L_{b})(L_{c}), Ir(Lₐ)₂(L_{b}), Ir(Lₐ)(L_{b})₂, Ir(Lₐ)₂(L_{c}), or Ir(Lₐ)(L_{c})₂.

According to an embodiment of the present disclosure, Lₐ has the structure represented by Formula 3 and comprises at least one structural unit selected from the group consisting of an aromatic ring formed by fusing a six-membered ring to a six-membered ring, a heteroaromatic ring formed by fusing a six-membered ring to a six-membered ring, an aromatic ring formed by fusing a six-membered ring to a five-membered ring, and a heteroaromatic ring formed by fusing a six-membered ring to a five-membered ring.

According to an embodiment of the present disclosure, in the organic electroluminescent device, Lₐ has the structure represented by Formula 3 and comprises at least one structural unit selected from the group consisting of naphthalene, phenanthrene, quinoline, isoquinoline, and azaphenanthrene.

According to an embodiment of the present disclosure, in the organic electroluminescent device, the phosphorescent material is an Ir complex and comprises the ligand Lₐ, wherein Lₐ is, at each occurrence identically or differently, selected from any one of the group consisting of the following structures:

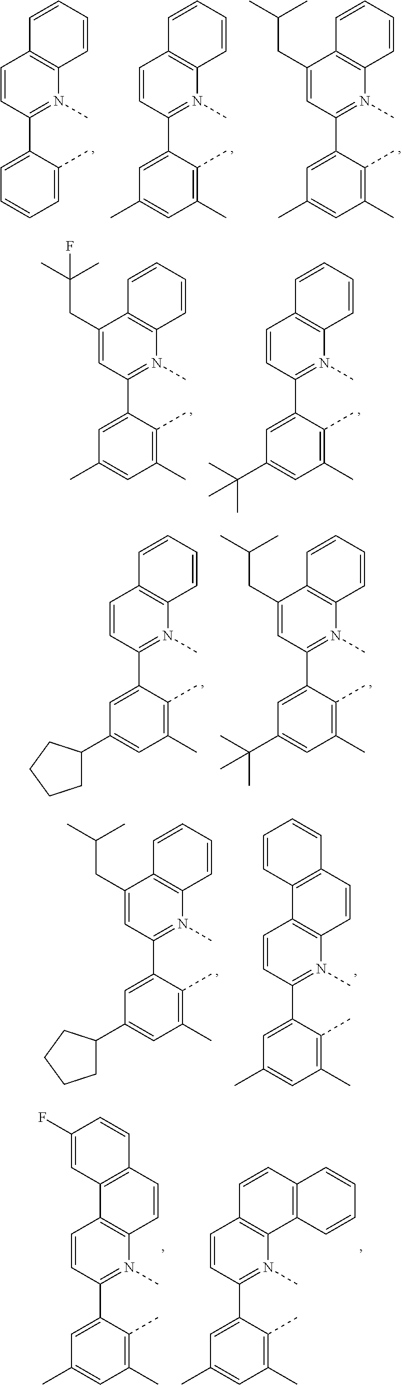

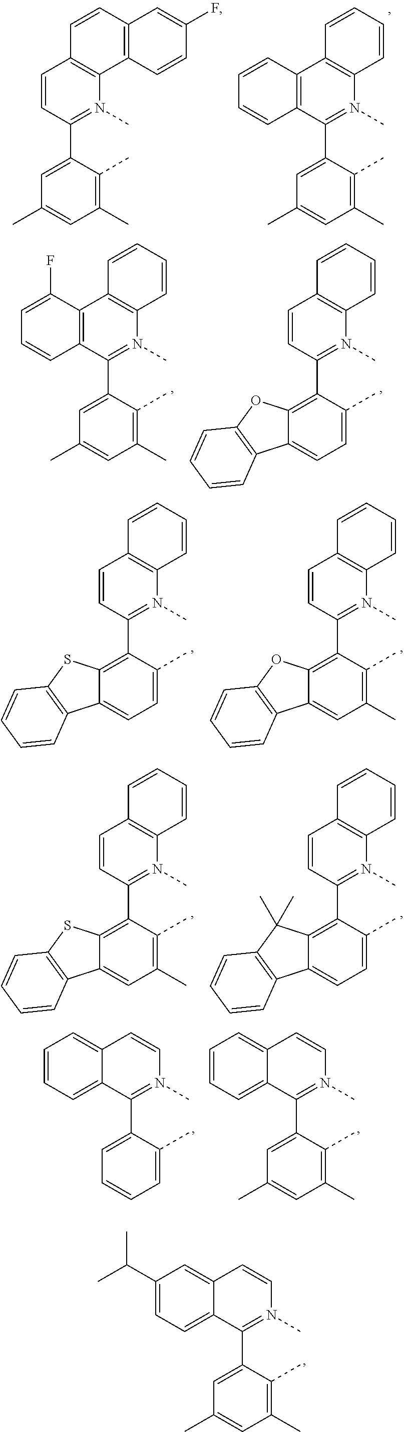

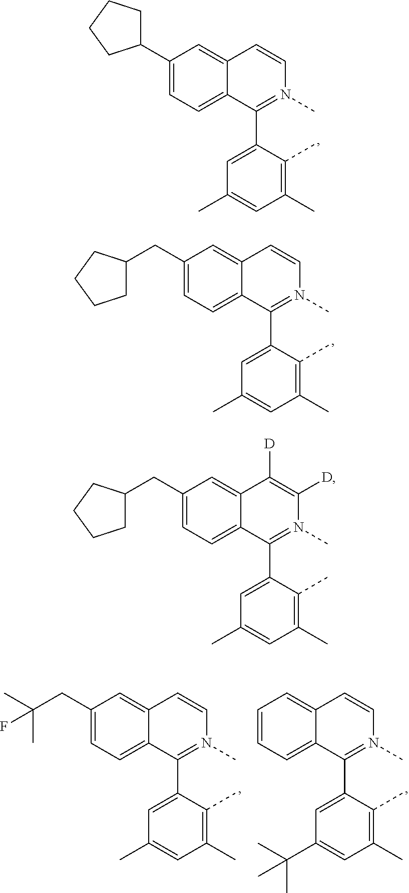

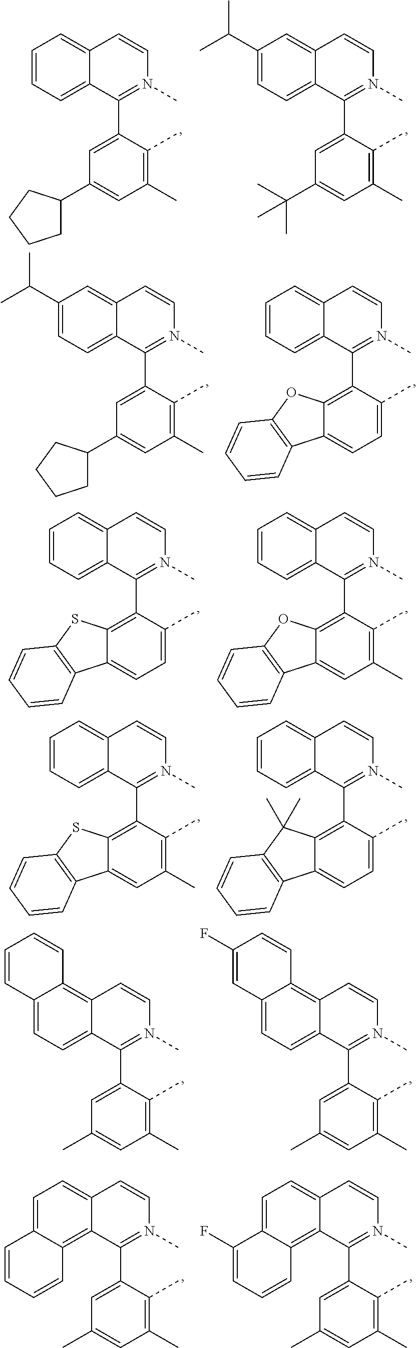

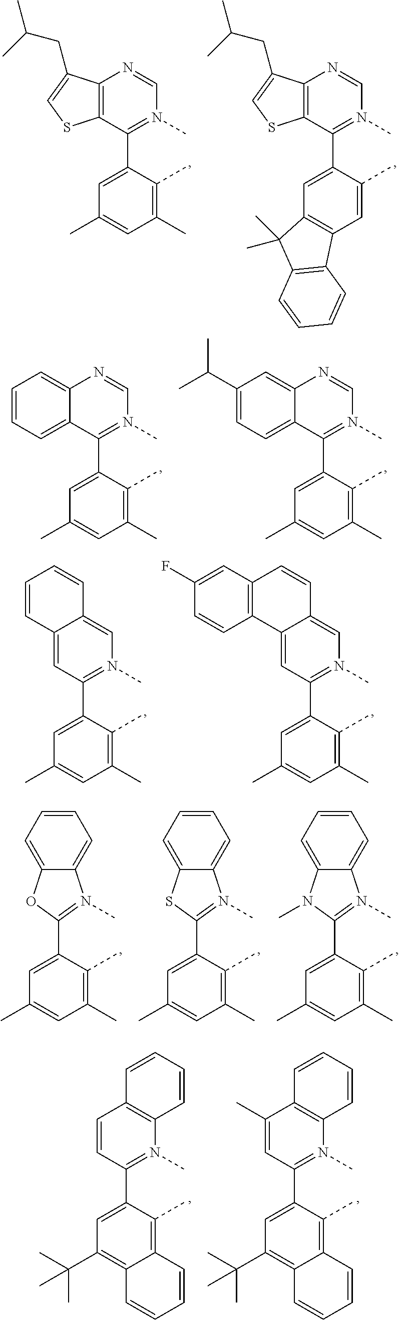

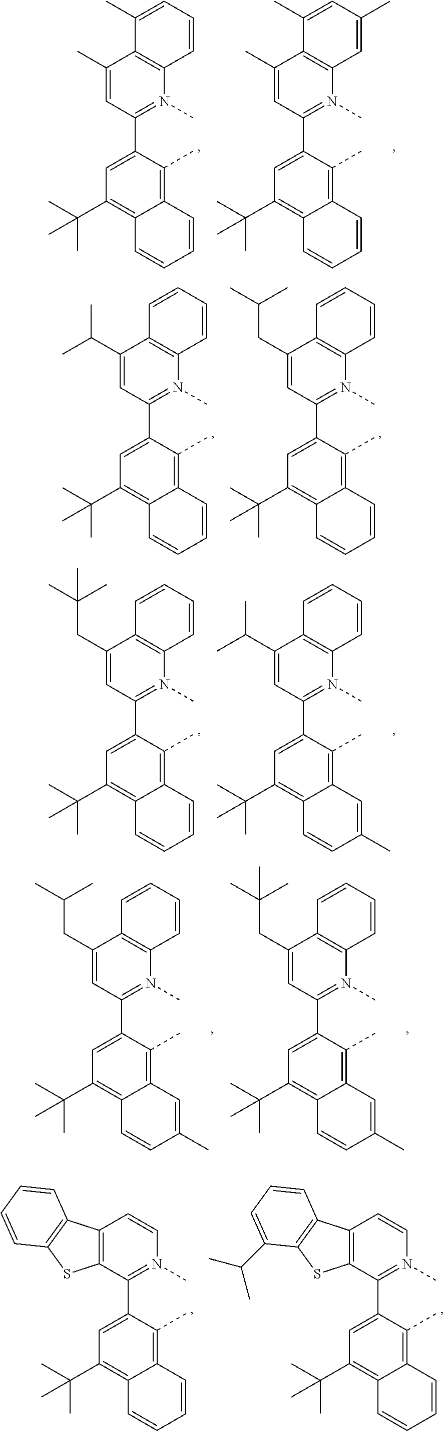

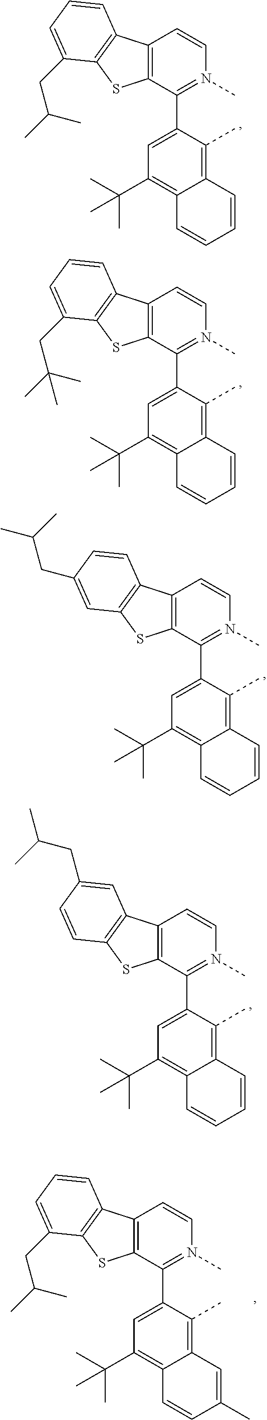

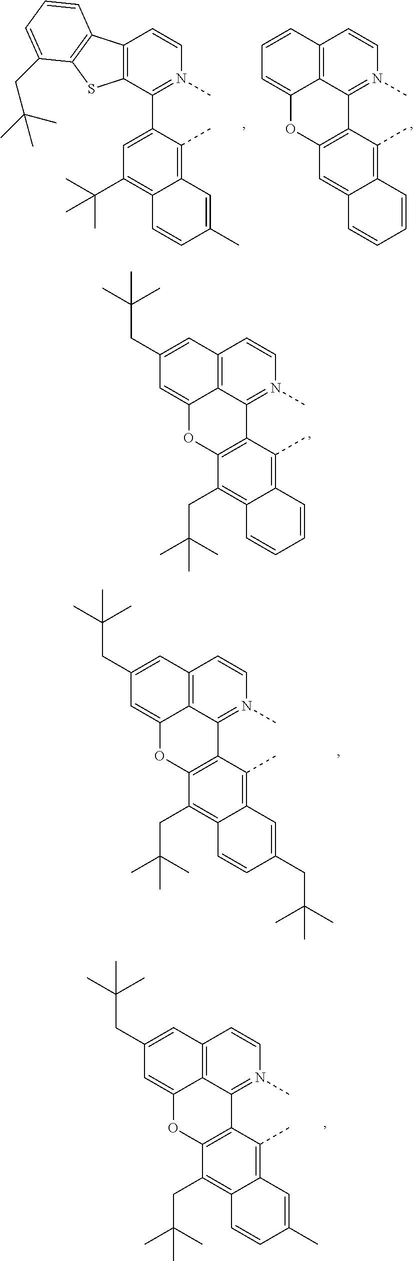

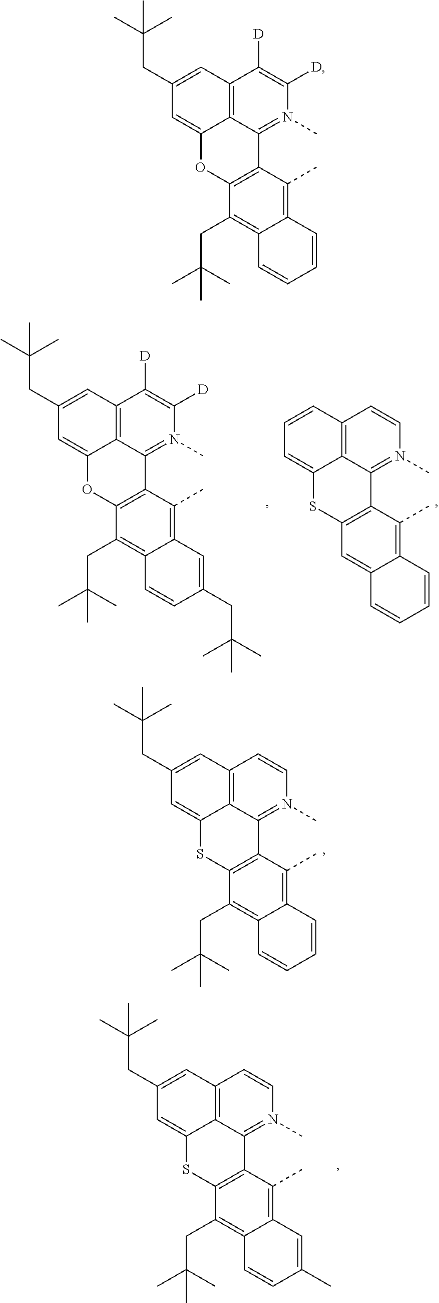

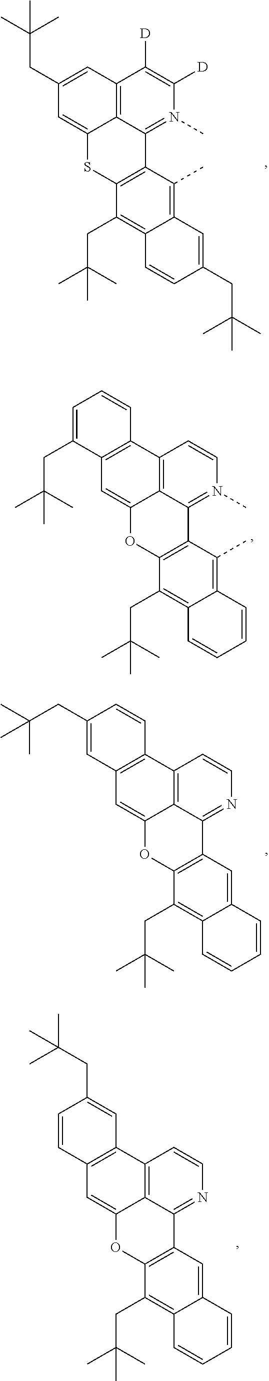

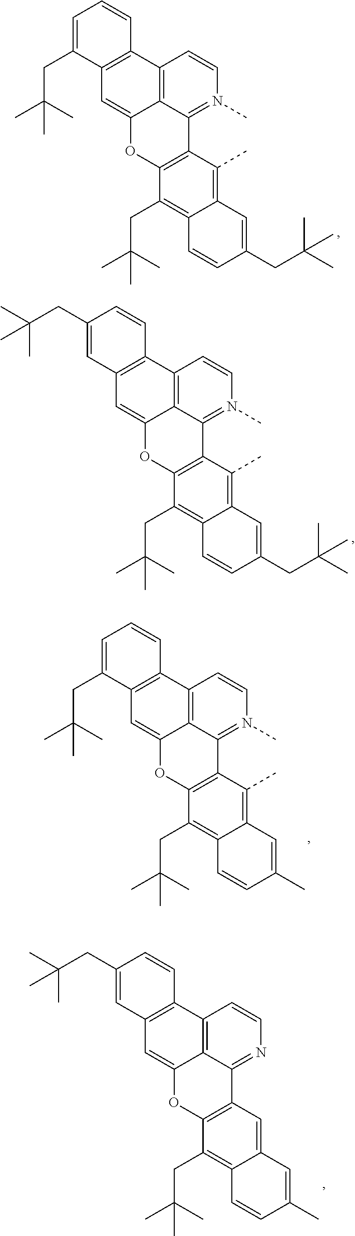

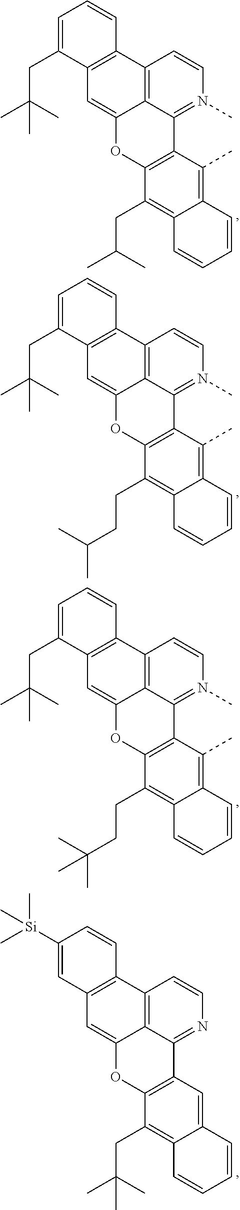

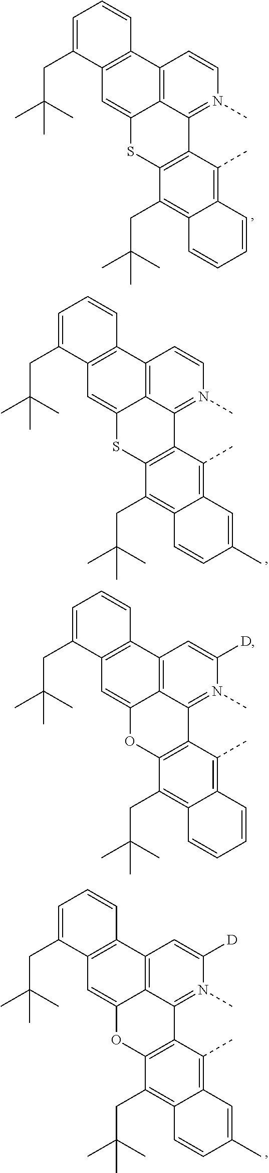

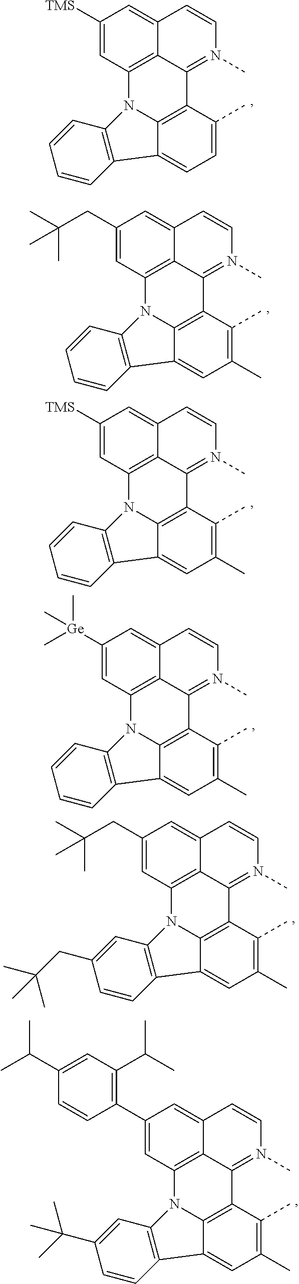

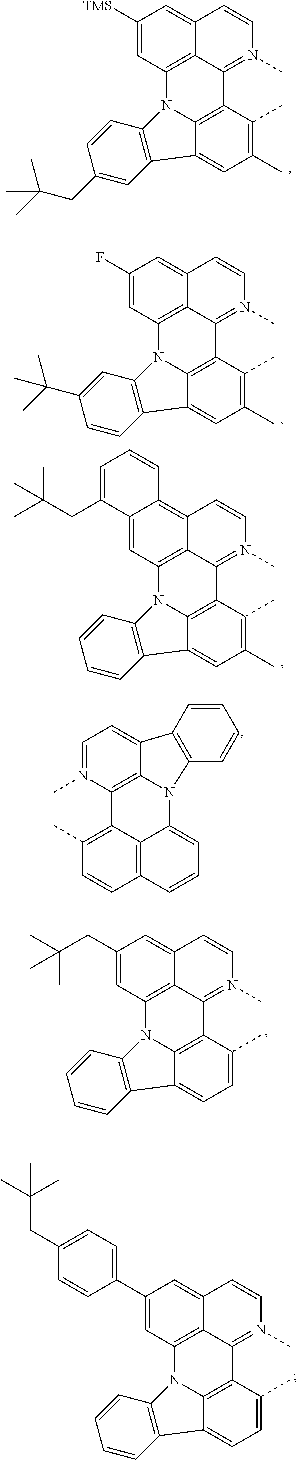

wherein in the above structures, TMS represents trimethylsilyl.

According to an embodiment of the present disclosure, in the organic electroluminescent device, the phosphorescent material is an Ir complex and comprises the ligand L_{b}, wherein L_{b} is, at each occurrence identically or differently, selected from any one of the group consisting of the following structures:

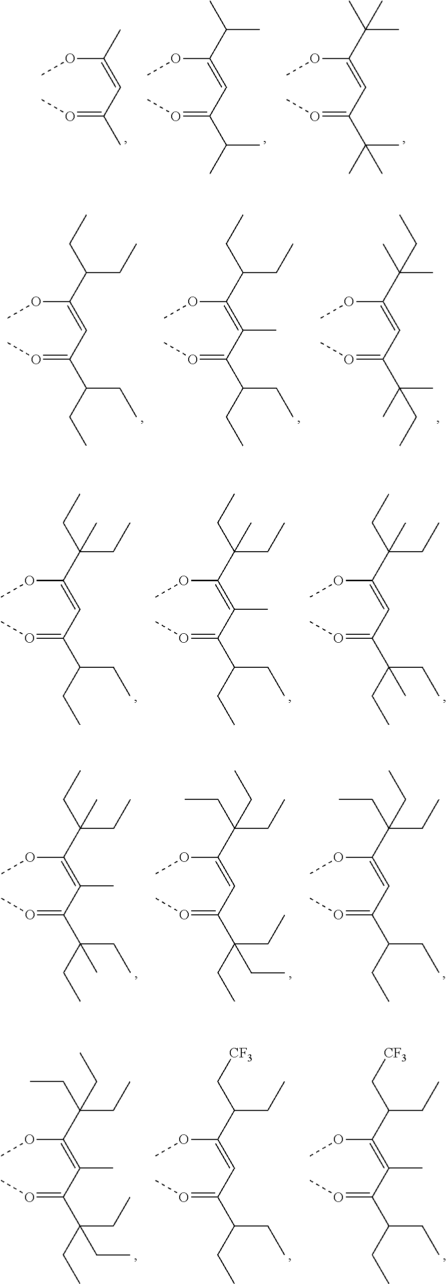

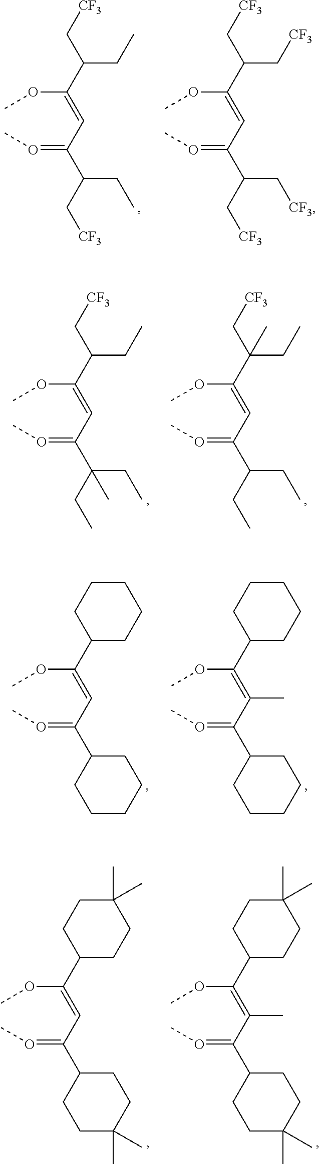

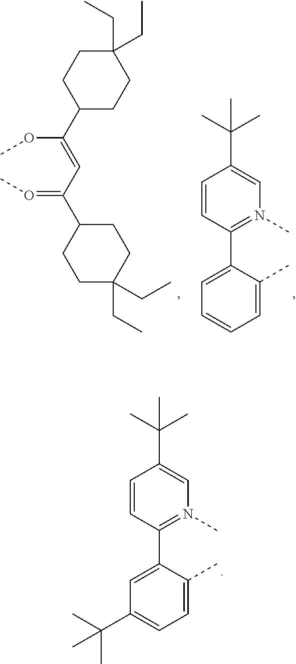

According to an embodiment of the present disclosure, in the organic electroluminescent device, the phosphorescent material is selected from the group consisting of the following structures:

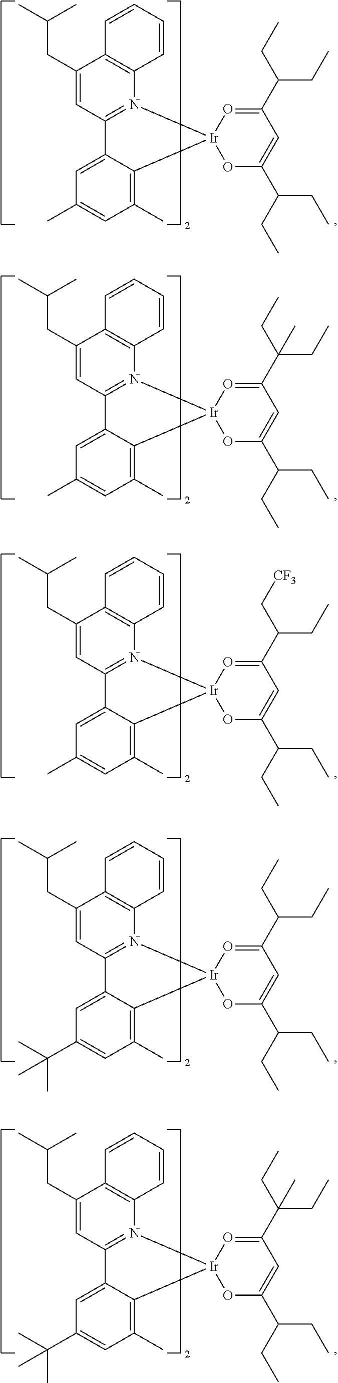

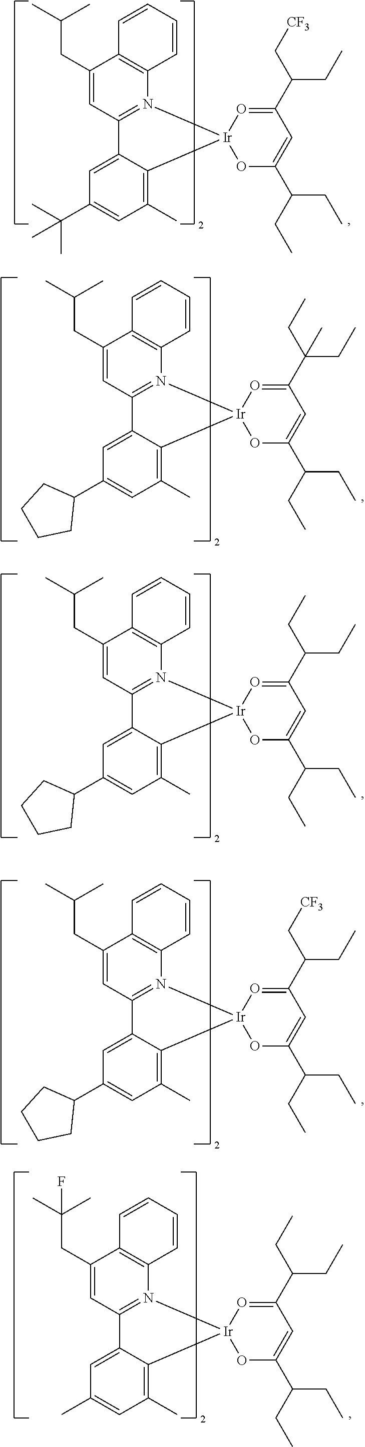

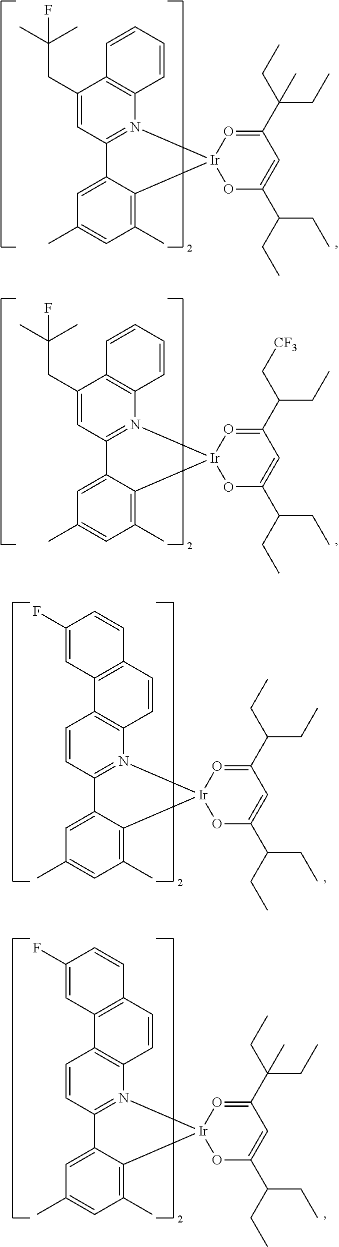

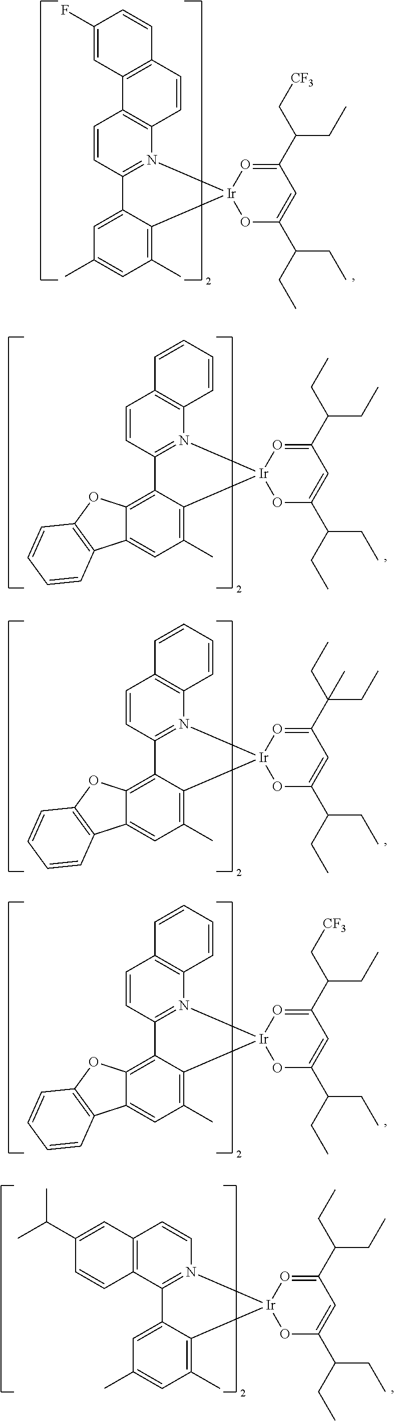

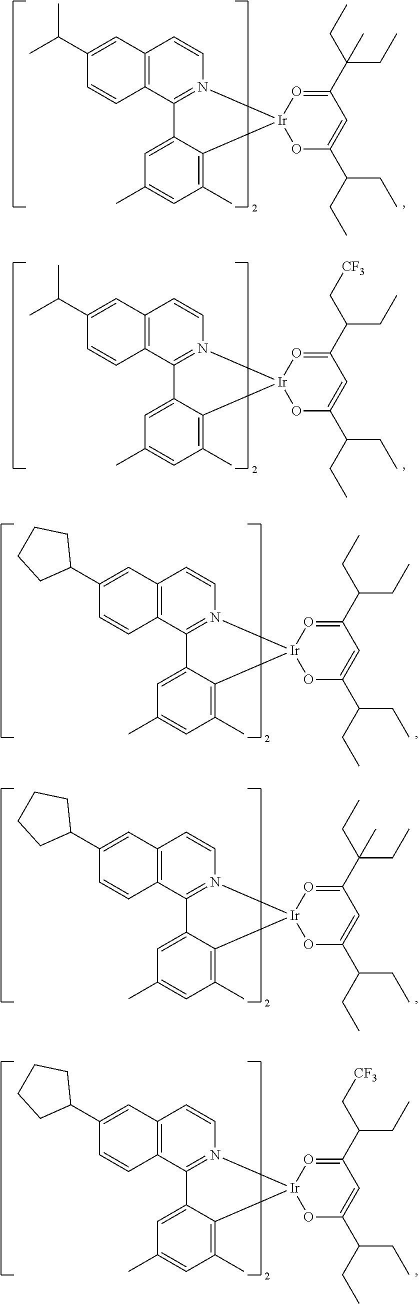

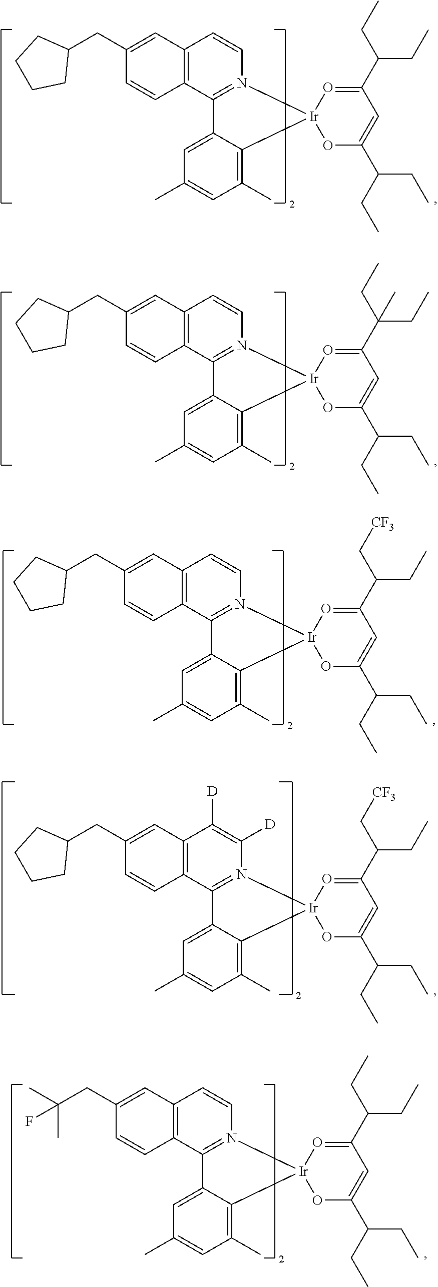

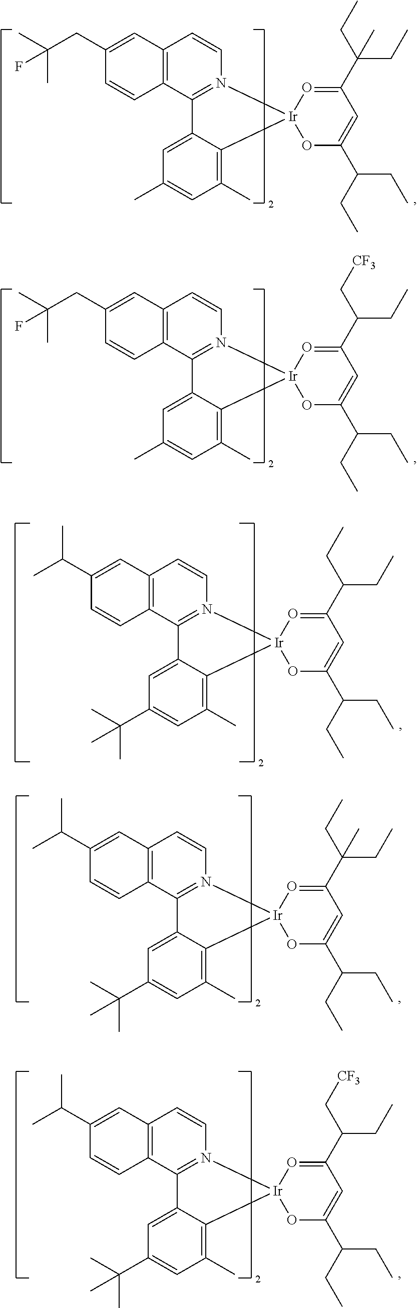

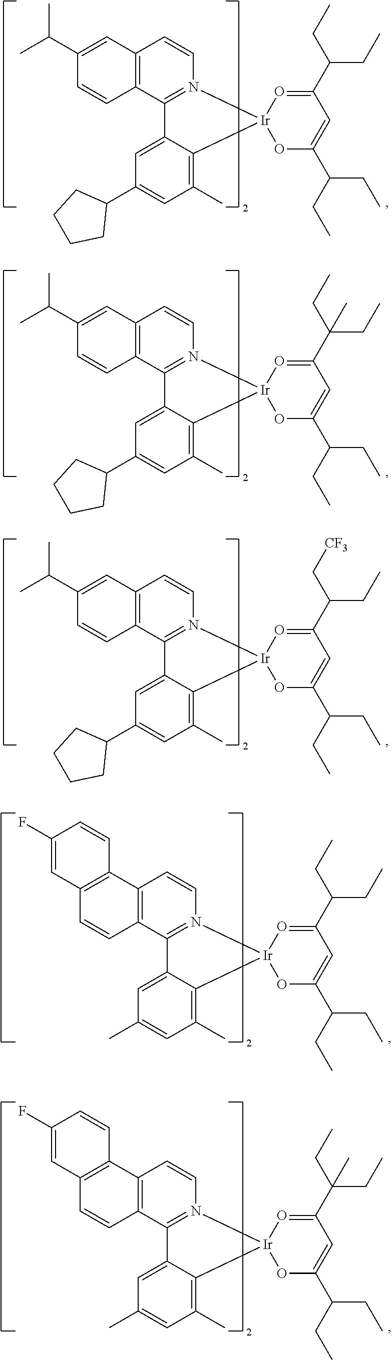

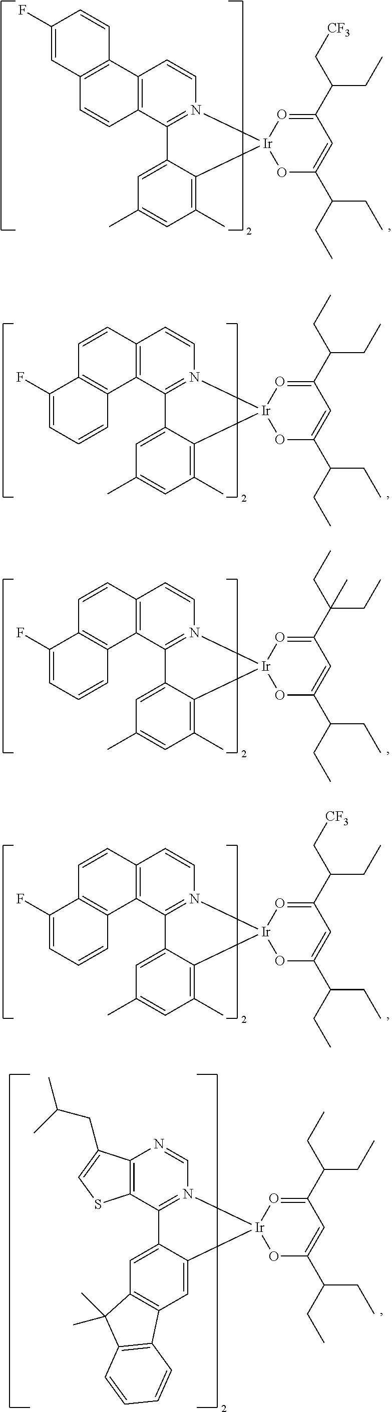

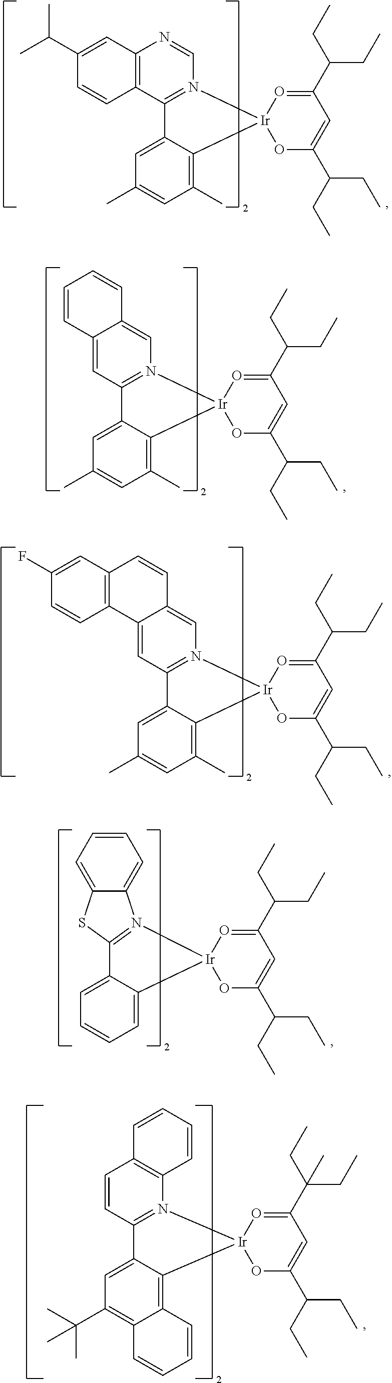

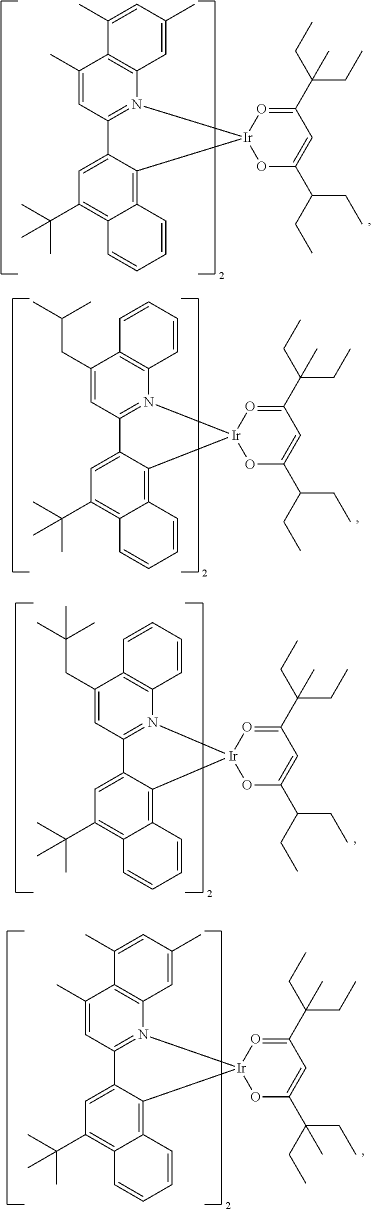

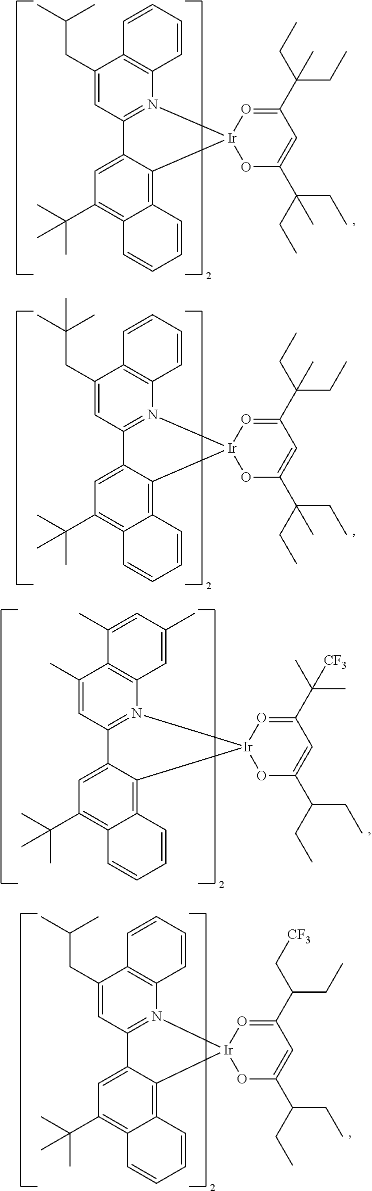

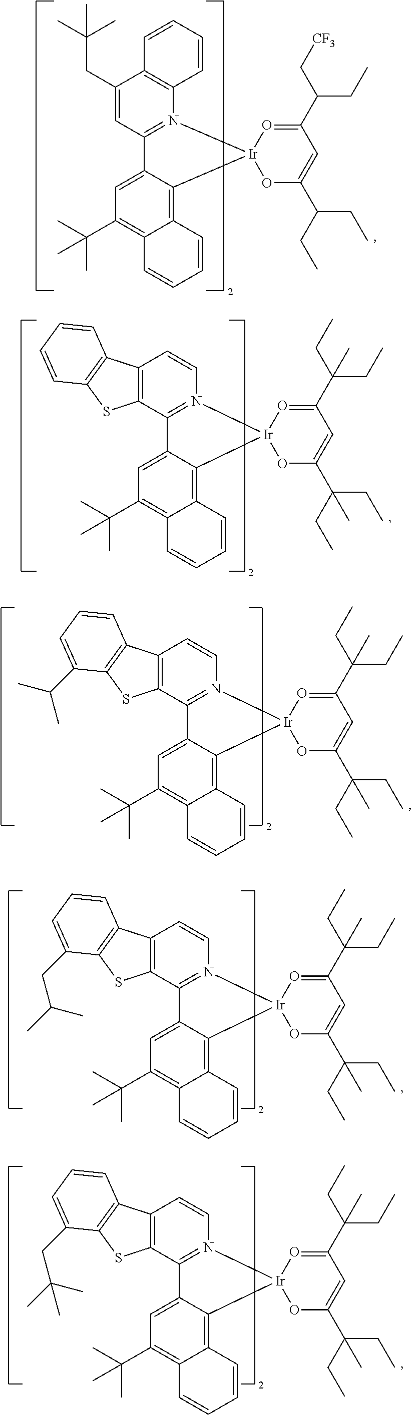

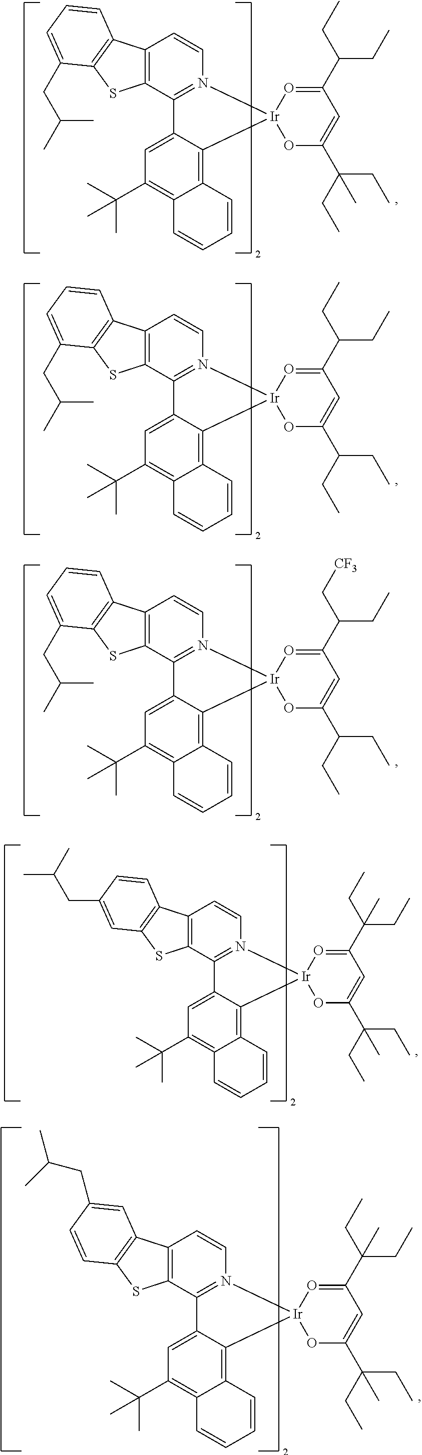

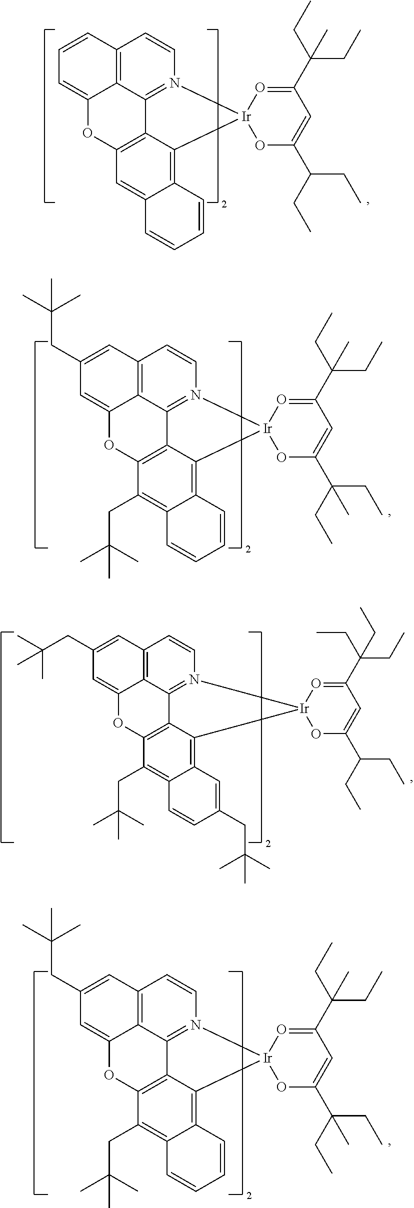

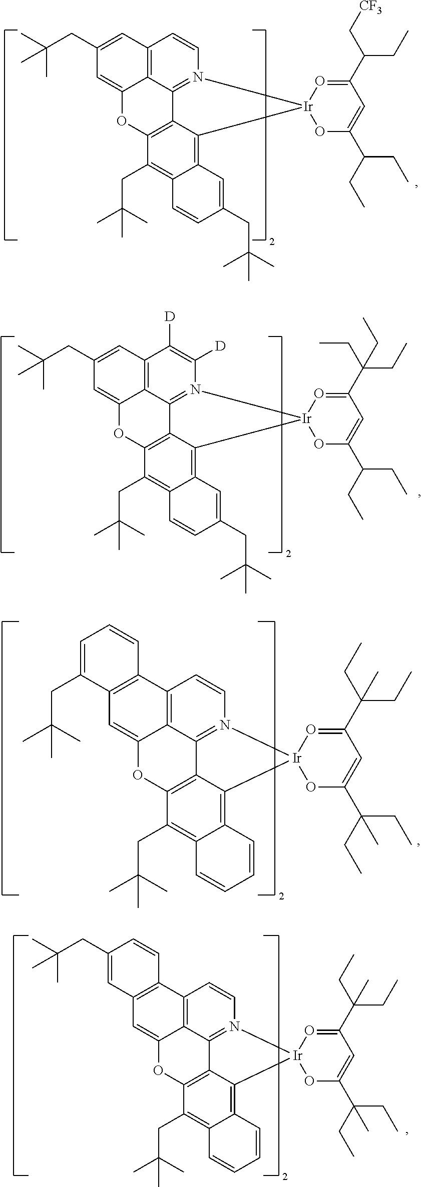

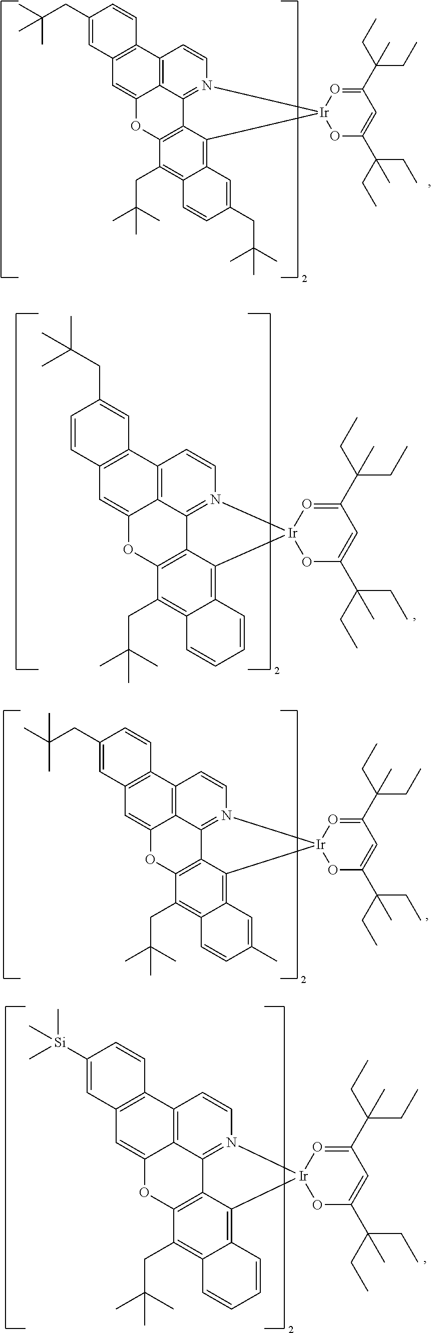

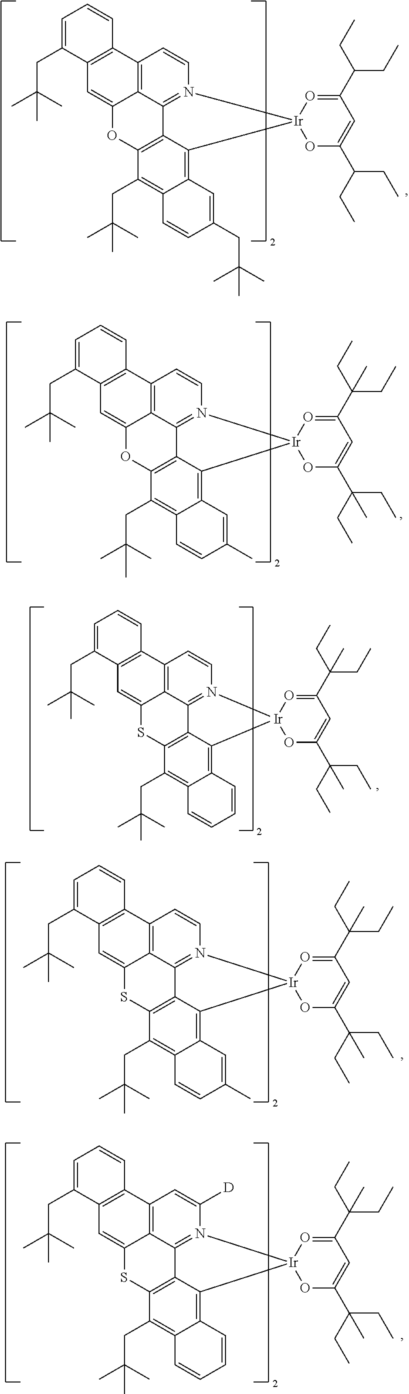

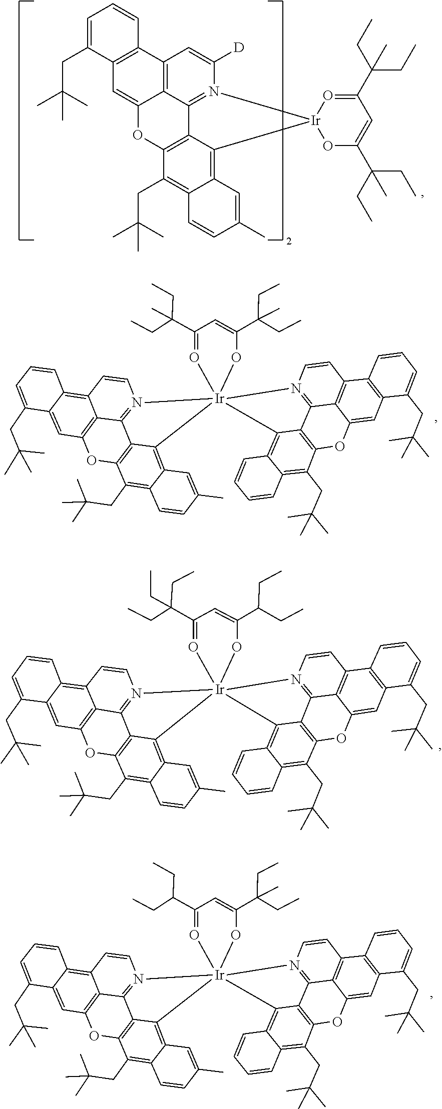

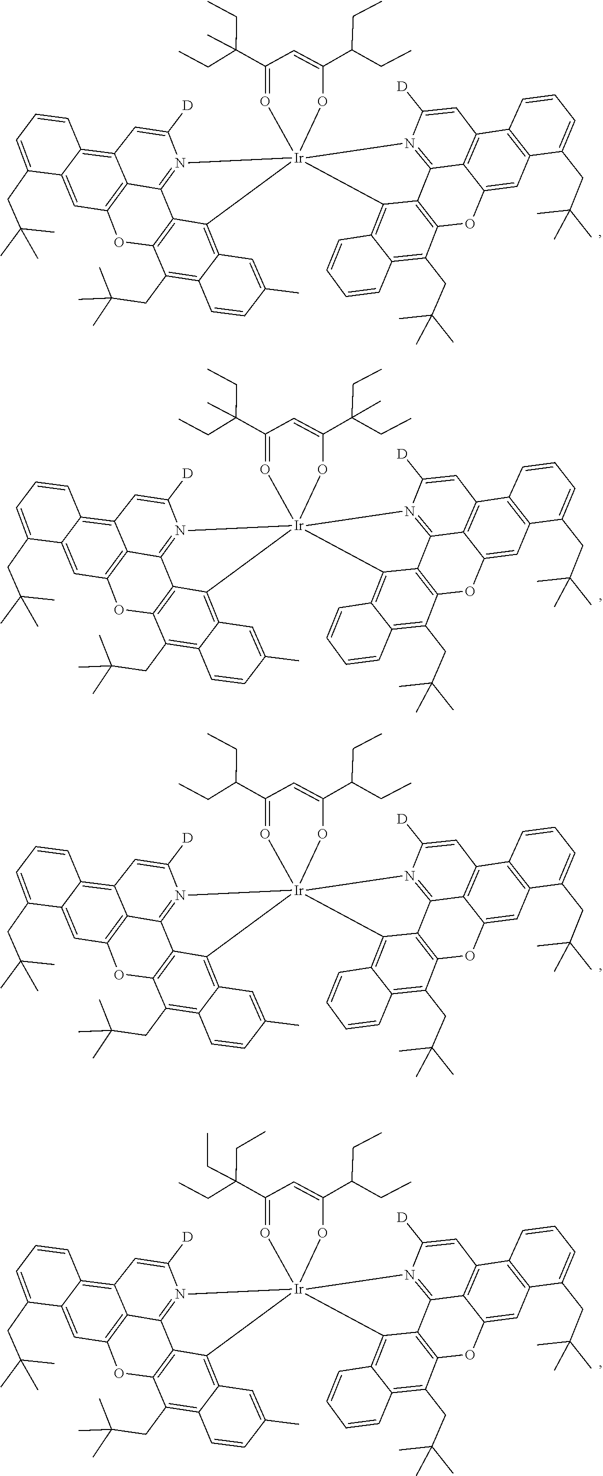

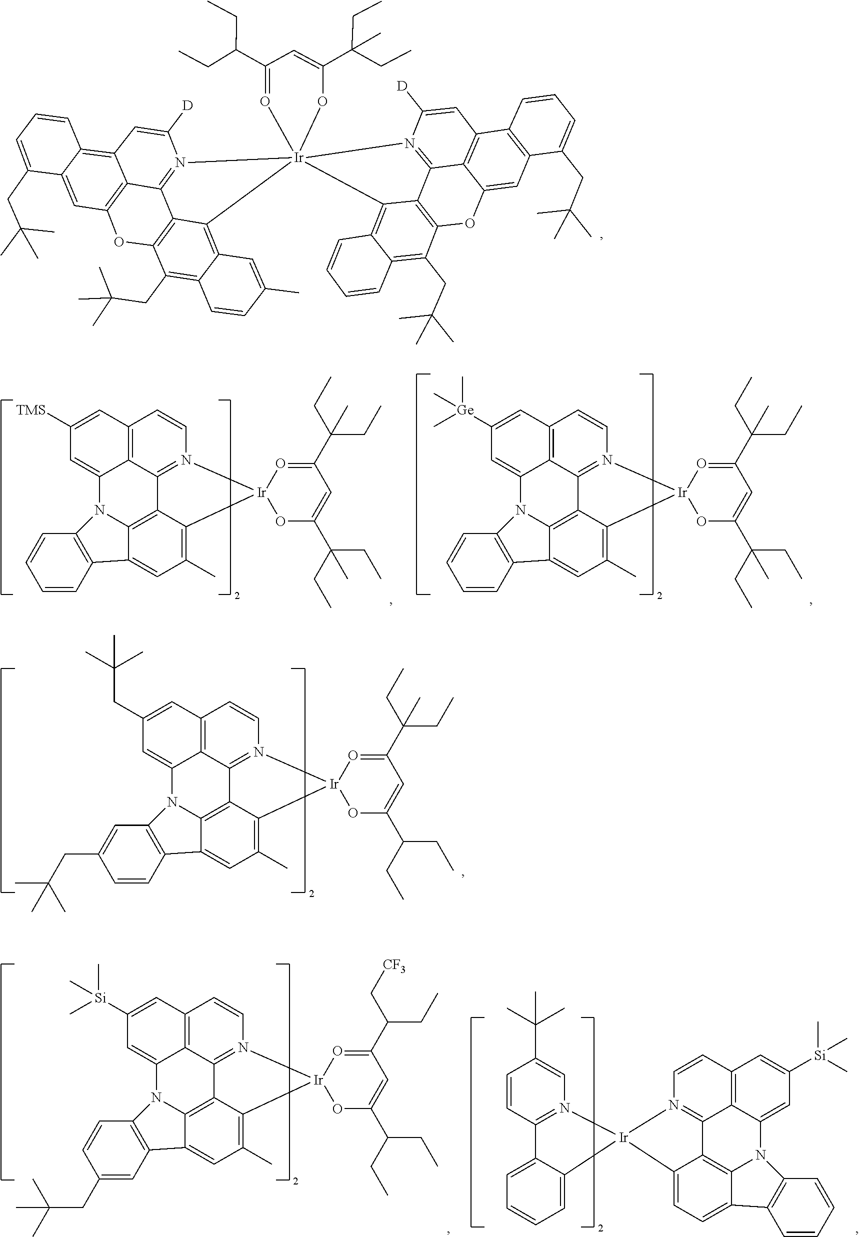

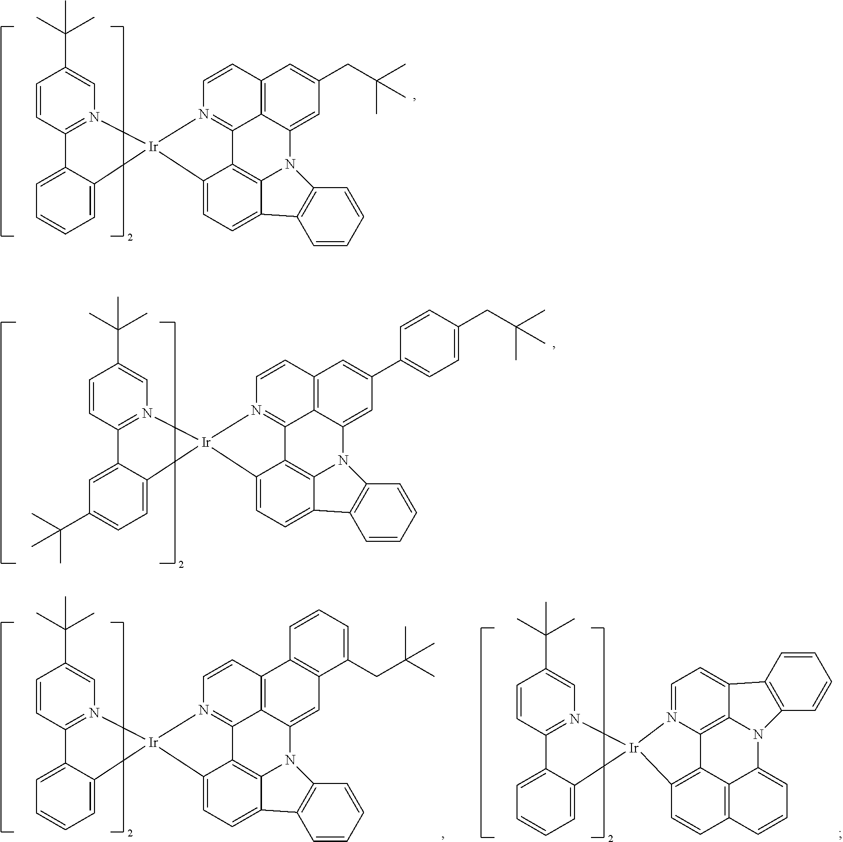

wherein in the above structures, TMS represents trimethylsilyl.

According to another embodiment of the present disclosure, further disclosed is a compound composition comprising a compound having a structure represented by Formula 1, wherein the compound has a specific structure described in any one of the preceding embodiments.

According to an embodiment of the present disclosure, the compound composition comprises a second compound having a structure represented by any one of Formula 2-1 to Formula 2-3:

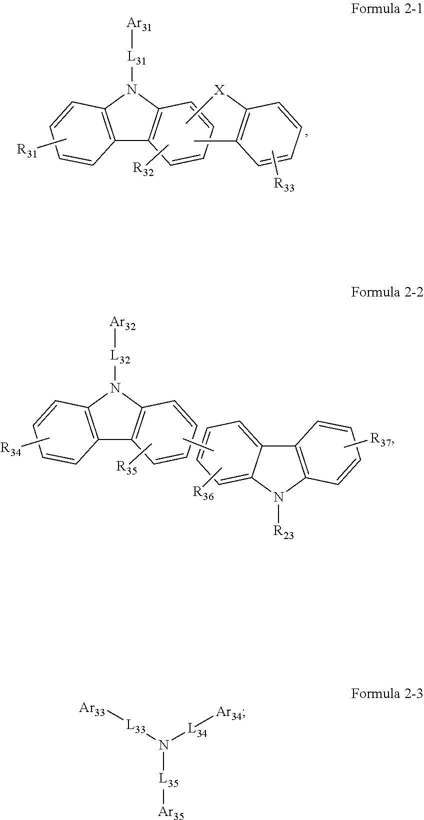

wherein
in Formula 2-1 to Formula 2-3, Ar₃₁ to Ar₃₅ are, at each occurrence identically or differently, selected from substituted or unsubstituted aryl having 6 to 30 carbon atoms or substituted or unsubstituted heteroaryl having 3 to 30 carbon atoms;
L₃₁ to L₃₅ are, at each occurrence identically or differently, selected from a single bond, substituted or unsubstituted arylene having 6 to 30 carbon atoms, substituted or unsubstituted heteroarylene having 3 to 30 carbon atoms, or a combination thereof;
X is, at each occurrence identically or differently, selected from O, S, CR₂₁R₂₂, or NR₂₃;
R₃₁ to R₃₇ represent, at each occurrence identically or differently, mono-substitution, multiple substitutions, or non-substitution;
R₃₁ to R₃₇ and R₂₁ to R₂₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₃₁ to R₃₇ can be optionally joined to form a ring.

According to another embodiment of the present disclosure, further disclosed is an electronic apparatus comprising an organic electroluminescent device, wherein the organic electroluminescent device has a specific structure described in any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, compounds disclosed herein may be used in combination with a wide variety of light-emitting dopants, hosts, transporting layers, blocking layers, injection layers, electrodes, and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well known to the persons skilled in the art. As the persons skilled in the art are aware of the above-mentioned equipment use, test methods, and other related contents, the inherent data of the sample can be obtained with certainty and without influence, so the above related contents are not further described in this patent.

### Material Synthesis Example

A method for preparing the compound of the present disclosure is not limited. Typically, the following compounds are used as examples without limitation, and synthesis routes and preparation methods thereof are described below.

### Synthesis Example 1: Synthesis of Compound 1-1

### Step 1: Synthesis of Intermediate 3

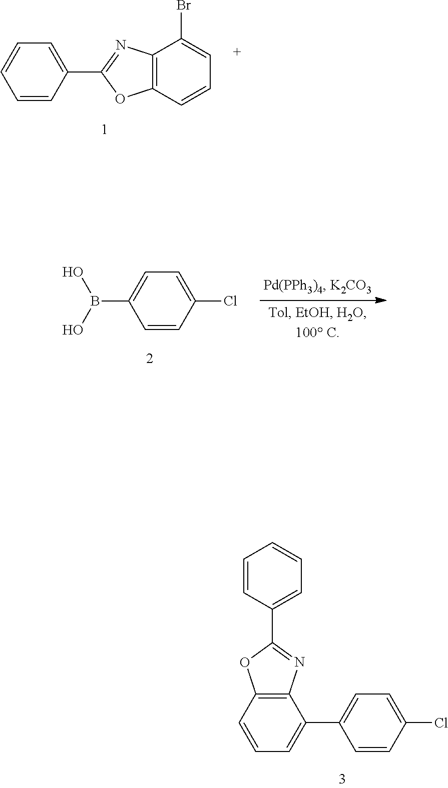 [SMILES: Brc1cccc2oc(-c3ccccc3)nc12 ; OB(O)c1ccc(Cl)cc1 ; Clc1ccc(-c2cccc3oc(-c4ccccc4)nc23)cc1]

Under nitrogen protection, Intermediate 1 (40.00 g, 145.92 mmol), Intermediate 2 (25.10 g, 160.51 mmol), tetrakis(triphenylphosphine)palladium (1.69 g, 1.46 mmol), potassium carbonate (40.33 g, 291.81 mmol), toluene (200 mL), ethanol (50 mL), and water (50 mL) were added to a three-necked flask and reacted for 16 h at 100° C. After the reaction was completed, the reaction solution was extracted with ethyl acetate. Organic phases were washed with water and concentrated to remove solvents. The crude product was purified through column chromatography (PE/DCM=1/1) to obtain Intermediate 3 as a white solid (35 g with a yield of 78%).

### Step 2: Synthesis of Intermediate 4

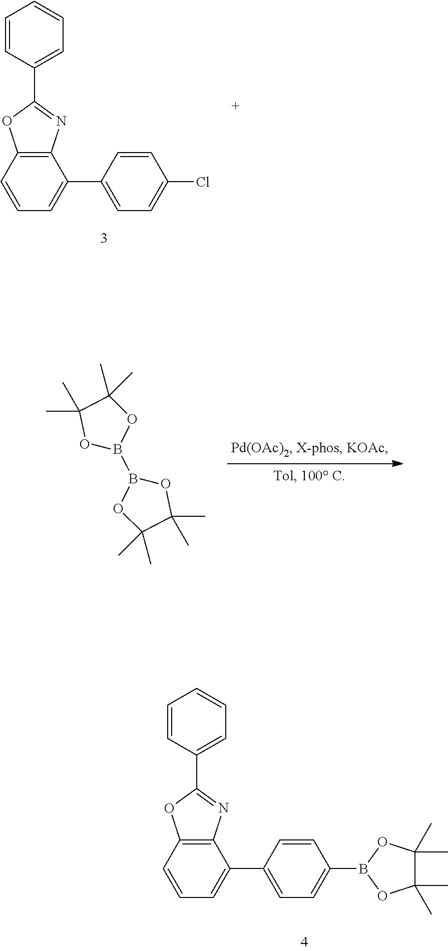

Under nitrogen protection, Intermediate 3 (5 g, 16.35 mmol), bis(pinacolato)diboron (10.38 g, 40.88 mmol), palladium acetate (183.56 mg, 0.82 mmol), X-Phos (779.58 mg, 1.64 mmol), potassium acetate (3.21 g, 32.71 mmol), and toluene (80 mL) were added to a three-necked flask, heated to 100° C., and reacted overnight. After the reaction was completed, the reaction solution was cooled, diluted with dichloromethane, and washed once with each of a saturated aqueous solution of sodium bicarbonate, water, and saturated brine. Organic phases were dried over anhydrous sodium sulfate, filtered, and evaporated to dryness. The crude product was purified through column chromatography (petroleum ether/dichloromethane=1/1) to obtain Intermediate 4 as a white solid compound (5 g with a yield of 77%).

### Step 3: Synthesis of Compound 1-1

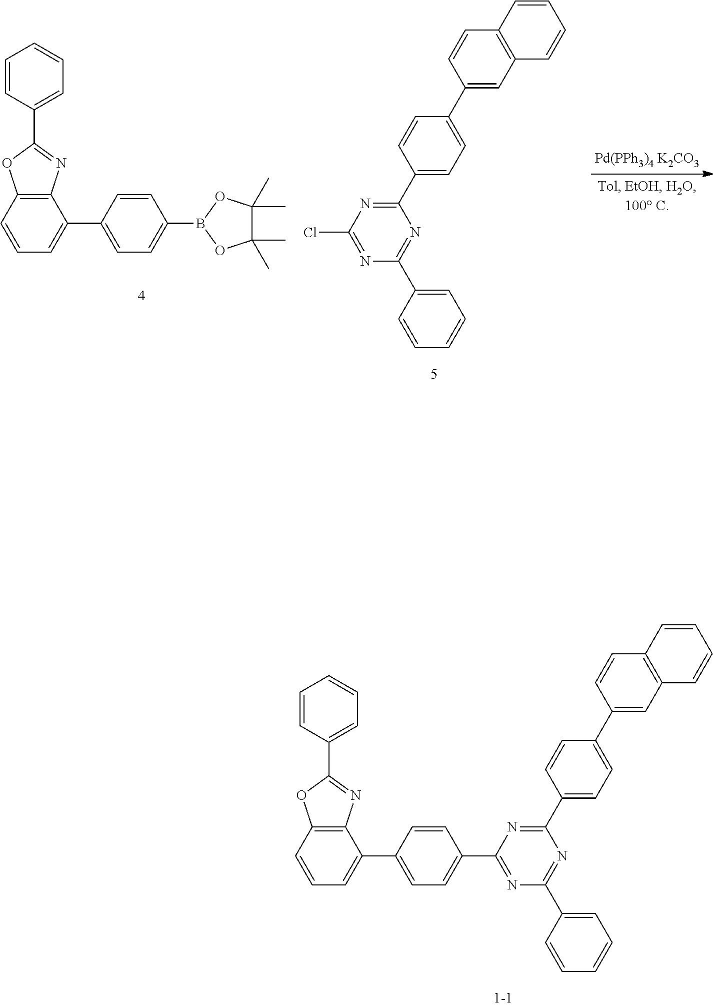

Under nitrogen protection, Intermediate 4 (3.0 g, 7.55 mmol), Intermediate 5 (3.57 g, 9.06 mmol), tetrakis(triphenylphosphine)palladium (436.31 mg, 0.38 mmol), potassium carbonate (2.09 g, 15.12 mmol), toluene (40 mL), ethanol (10 mL), and water (10 mL) were added to a three-necked flask and reacted overnight at 100° C. After the reaction was completed, the reaction solution was added with water to precipitate a solid and filtered to obtain the solid. The crude product was recrystallized from toluene to obtain Compound 1-1 as a white solid (1.8 g with a yield of 38%). The product was confirmed as the target product with a molecular weight of 628.23.

### Synthesis Example 2: Synthesis of Compound 1-215

### Step 1: Synthesis of Intermediate 7

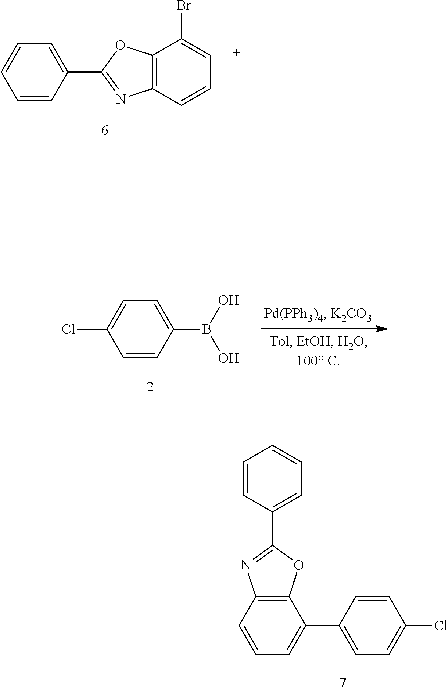 [SMILES: Brc1cccc2nc(-c3ccccc3)oc12 ; OB(O)c1ccc(Cl)cc1 ; Clc1ccc(-c2cccc3nc(-c4ccccc4)oc23)cc1]

Under nitrogen protection, Intermediate 6 (5.00 g, 18.24 mmol), Intermediate 2 (3.42 g, 21.87 mmol), tetrakis(triphenylphosphine)palladium (420 mg, 0.36 mmol), potassium carbonate (5.04 g, 36.47 mmol), toluene (100 mL), ethanol (20 mL), and water (20 mL) were added to a three-necked flask and reacted for 16 h at 100° C. After the reaction was completed, the reaction solution was extracted with ethyl acetate. Organic phases were washed with water and concentrated to remove solvents. The crude product was purified through column chromatography (PE/DCM=1/1) to obtain Intermediate 7 as a white solid (5 g with a yield of 90%).

### Step 2: Synthesis of Intermediate 8

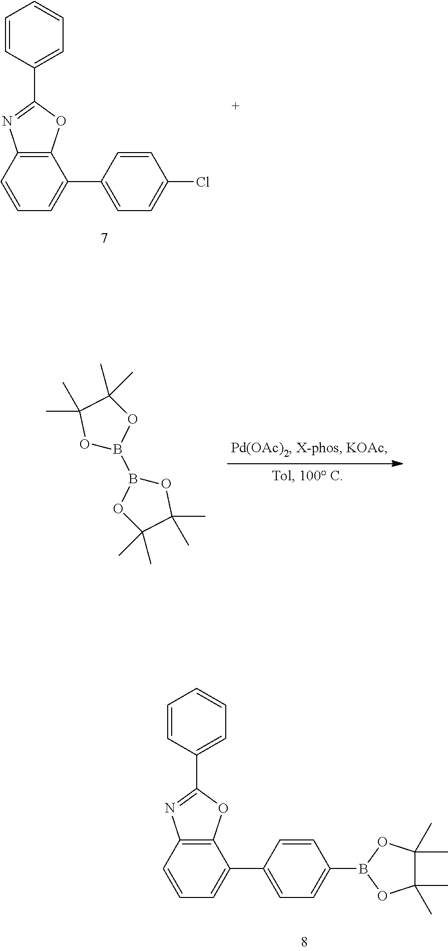

Under nitrogen protection, Intermediate 7 (5 g, 16.35 mmol), bis(pinacolato)diboron (10.38 g, 40.88 mmol), palladium acetate (183.56 mg, 0.82 mmol), X-Phos (779.58 mg, 1.64 mmol), potassium acetate (3.21 g, 32.71 mmol), and toluene (80 mL) were added to a three-necked flask, heated to 100° C., and reacted overnight. After the reaction was completed, the reaction solution was cooled, diluted with dichloromethane, and washed once with each of a saturated aqueous solution of sodium bicarbonate, water, and saturated brine. Organic phases were dried over anhydrous sodium sulfate, filtered, and evaporated to dryness. The crude product was purified through column chromatography (petroleum ether/dichloromethane=1/1) to obtain Intermediate 8 as a white solid compound (4.5 g with a yield of 69%).

### Step 3: Synthesis of Compound 1-215

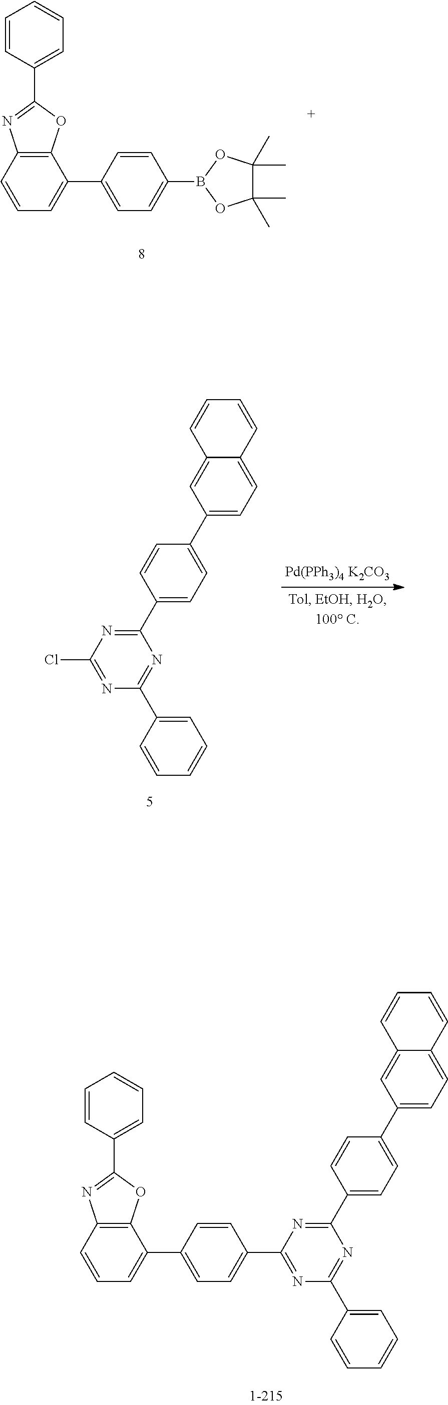 [SMILES: CC1(C)OB(c2ccc(-c3cccc4nc(-c5ccccc5)oc34)cc2)OC1(C)C ; Clc1nc(-c2ccccc2)nc(-c2ccc(-c3ccc4ccccc4c3)cc2)n1 ; c1ccc(-c2nc(-c3ccc(-c4ccc5ccccc5c4)cc3)nc(-c3ccc(-c4cccc5nc(-c6ccccc6)oc45)cc3)n2)cc1]

Under nitrogen protection, Intermediate 8 (3.0 g, 7.55 mmol), Intermediate 5 (3.57 g, 9.06 mmol), tetrakis(triphenylphosphine)palladium (436.31 mg, 0.38 mmol), potassium carbonate (2.09 g, 15.12 mmol), toluene (40 mL), ethanol (10 mL), and water (10 mL) were added to a three-necked flask and reacted overnight at 100° C. After the reaction was completed, the reaction solution was added with water to precipitate a solid and filtered to obtain the solid. The crude product was recrystallized from toluene to obtain Compound 1-215 as a white solid (2.6 g with a yield of 55%). The product was confirmed as the target product with a molecular weight of 628.23.

### Synthesis Example 3: Synthesis of Compound 1-2

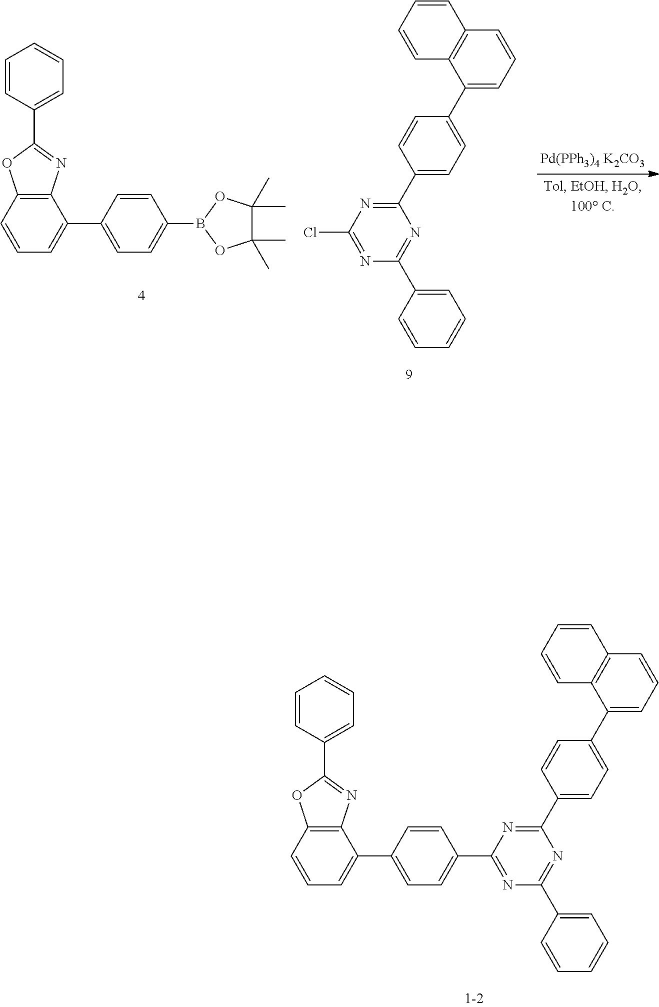

Under nitrogen protection, Intermediate 4 (3.0 g, 7.55 mmol), Intermediate 9 (3.57 g, 9.06 mmol), tetrakis(triphenylphosphine)palladium (436.31 mg, 0.38 mmol), potassium carbonate (2.09 g, 15.12 mmol), toluene (40 mL), ethanol (10 mL), and water (10 mL) were added to a three-necked flask and reacted overnight at 100° C. After the reaction was completed, the reaction solution was added with water to precipitate a solid and filtered to obtain the solid. The crude product was recrystallized from toluene to obtain Compound 1-2 as a white solid (3.2 g with a yield of 67%). The product was confirmed as the target product with a molecular weight of 628.23.

### Synthesis Example 4: Synthesis of Compound 1-224

Step 1: Synthesis of Intermediate 11

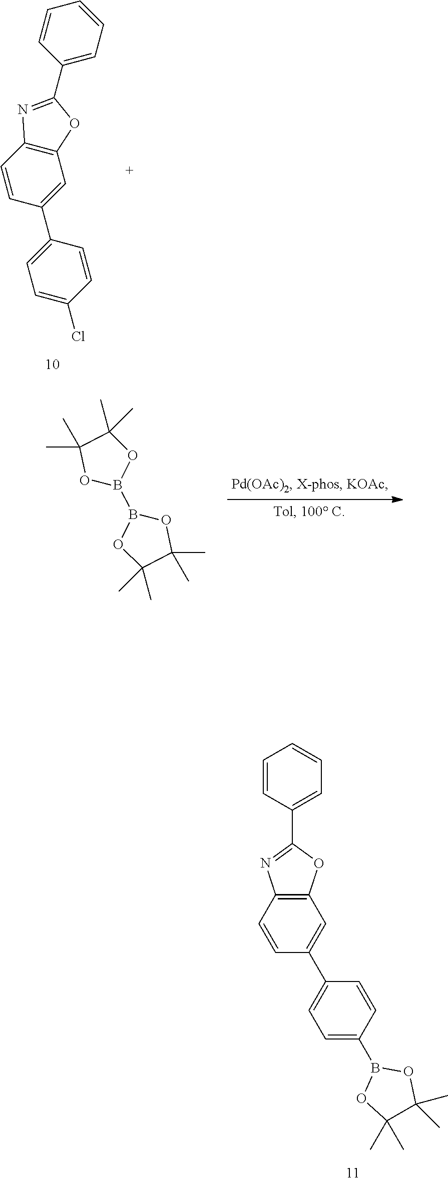

Under nitrogen protection, Intermediate 10 (3.9 g, 12.76 mmol), bis(pinacolato)diboron (8.1 g, 31.89 mmol), palladium acetate (143.18 mg, 0.64 mmol), X-Phos (608.07 mg, 1.28 mmol), potassium acetate (2.5 g, 25.47 mmol), and toluene (80 mL) were added to a three-necked flask, heated to 100° C., and reacted overnight. After the reaction was completed, the reaction solution was cooled, diluted with dichloromethane, and washed once with each of a saturated aqueous solution of sodium bicarbonate, water, and saturated brine. Organic phases were dried over anhydrous sodium sulfate, filtered, and evaporated to dryness. The crude product was purified through column chromatography (petroleum ether/dichloromethane=1/1) to obtain Intermediate 11 as a white solid compound (4.8 g with a yield of 95%).

### Step 2: Synthesis of Compound 1-224

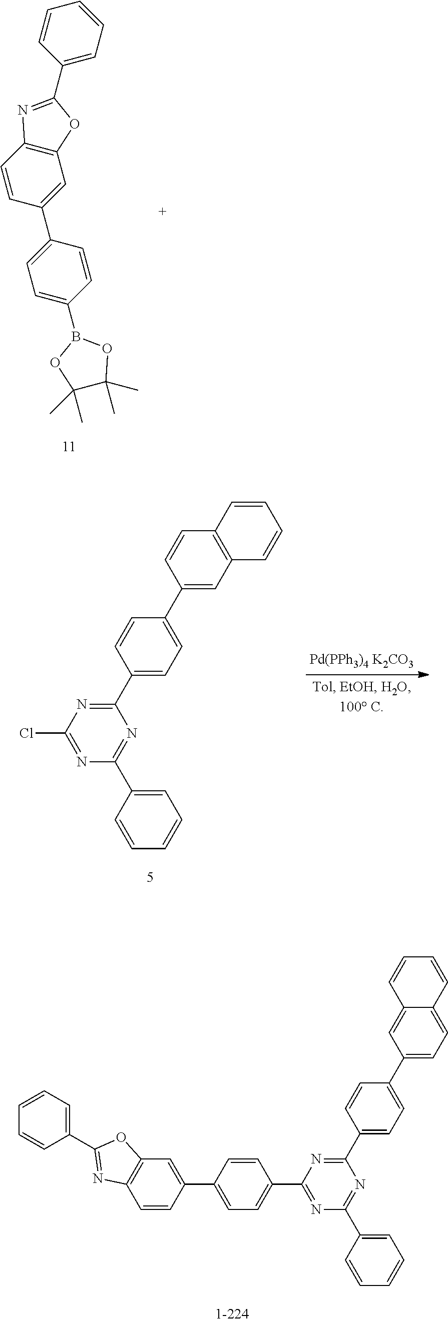 [SMILES: CC1(C)OB(c2ccc(-c3ccc4nc(-c5ccccc5)oc4c3)cc2)OC1(C)C ; Clc1nc(-c2ccccc2)nc(-c2ccc(-c3ccc4ccccc4c3)cc2)n1 ; c1ccc(-c2nc(-c3ccc(-c4ccc5ccccc5c4)cc3)nc(-c3ccc(-c4ccc5nc(-c6ccccc6)oc5c4)cc3)n2)cc1]

Under nitrogen protection, Intermediate 11 (3 g, 7.55 mmol), Intermediate 5 (3.57 g, 9.06 mmol), tetrakis(triphenylphosphine)palladium (436.31 mg, 0.38 mmol), potassium carbonate (2.09 g, 15.12 mmol), toluene (40 mL), ethanol (10 mL), and water (10 mL) were added to a three-necked flask and reacted overnight at 100° C. After the reaction was completed, the reaction solution was added with water to precipitate a solid and filtered to obtain the solid. The crude product was recrystallized from toluene to obtain Compound 1-224 as a white solid (2.2 g with a yield of 46%). The product was confirmed as the target product with a molecular weight of 628.23.

Those skilled in the art will appreciate that the above preparation methods are merely exemplary. Those skilled in the art can obtain other compound structures of the present disclosure through the modifications of the preparation methods.

### Device Example

### Device Example 1

Firstly, a glass substrate having an indium tin oxide (ITO) anode with a thickness of 120 nm was cleaned and then treated with UV ozone and oxygen plasma. After the treatment, the substrate was dried in a nitrogen-filled glovebox to remove moisture and then mounted on a substrate holder and placed in a vacuum chamber. Organic layers specified below were sequentially deposited through vacuum thermal evaporation on the ITO anode at a rate of 0.01-5 Å/s and a vacuum degree of about 10⁻⁶ Torr. Compounds HT and HI were co-deposited at a weight ratio of 97:3 for use as a hole injection layer (HIL) with a thickness of 100 Å. Compound HT was used as a hole transporting layer (HTL) with a thickness of 400 Å. Compound EB was used as an electron blocking layer (EBL) with a thickness of 50 Å. Then, Compound 1-215 of the present disclosure as a first host, Compound H-137 as a second host, and Compound RD as a dopant were co-deposited at a weight ratio of 48.5:48.5:3 for use as an emissive layer (EML) with a thickness of 400 Å. Compound HB was used as a hole blocking layer (HBL) with a thickness of 50 Å. On the HBL, Compound ET and 8-hydroxyquinolinolato-lithium (Liq) were co-deposited at (a weight ratio of 40:60) as an electron transporting layer (ETL) with a thickness of 350 Å. Finally, 8-hydroxyquinolinolato-lithium (Liq) was deposited for use as an electron injection layer (EIL) with a thickness of 10 Å and Al was deposited for use as a cathode with a thickness of 1200 Å. The device was transferred back to the glovebox and encapsulated with a glass lid to complete the device.

### Device Example 2

Device Example 2 was implemented in the same manner as Device Example 1 except that Compound 1-215 of the present disclosure was replaced with Compound 1-224 of the present disclosure as the first host in the emissive layer (EML).

### Device Example 3

Device Example 3 was implemented in the same manner as Device Example 1 except that Compound 1-215 of the present disclosure was replaced with Compound 1-1 of the present disclosure as the first host in the emissive layer (EML).

### Device Example 4

Device Example 4 was implemented in the same manner as Device Example 1 except that Compound 1-215 of the present disclosure was replaced with Compound 1-2 of the present disclosure as the first host in the emissive layer (EML).

### Device Comparative Example 1

Device Comparative Example 1 was implemented in the same manner as Device Example 1 except that Compound 1-215 of the present disclosure was replaced with Compound A as the first host in the emissive layer (EML).

### Device Comparative Example 2

Device Comparative Example 2 was implemented in the same manner as Device Example 1 except that Compound 1-215 of the present disclosure was replaced with Compound B as the first host in the emissive layer (EML).

Detailed structures and thicknesses of layers of the devices are shown in the following table. A layer using more than one material is obtained by doping different compounds at their weight ratio as recorded.

TABLE 1
Device structures of device examples and comparative examples

| Device ID | HIL | HTL | EBL | EML | HBL | ETL |
|---|---|---|---|---|---|---|
| Example 1 | Compound | Compound | Compound | Compound 1- | Compound | Compound |
| | HT:Compound | HT | EB | 215:Compound H- | HB | ET:Liq |
| | HI (97:3) | (400 Å) | (50 Å) | 137:Compound RD | (50 Å) | (40:60) |
| | (100 Å) | | | (48.5:48.5:3) (400 Å) | | (350 Å) |
| Example 2 | Compound | Compound | Compound | Compound 1- | Compound | Compound |
| | HT:Compound | HT | EB | 224:Compound H- | HB | ET:Liq |
| | HI (97:3) | (400 Å) | (50 Å) | 137:Compound RD | (50 Å) | (40:60) |
| | (100 Å) | | | (48.5:48.5:3) (400 Å) | | (350 Å) |
| Example 3 | Compound | Compound | Compound | Compound 1- | Compound | Compound |
| | HT:Compound | HT | EB | 1:Compound H- | HB | ET:Liq |
| | HI (97:3) | (400 Å) | (50 Å) | 137:Compound RD | (50 Å) | (40:60) |
| | (100 Å) | | | (48.5:48.5:3) (400 Å) | | (350 Å) |
| Example 4 | Compound | Compound | Compound | Compound 1- | Compound | Compound |
| | HT:Compound | HT | EB | 2:Compound H- | HB | ET:Liq |
| | HI (97:3) | (400 Å) | (50 Å) | 137:Compound RD | (50 Å) | (40:60) |
| | (100 Å) | | | (48.5:48.5:3) (400 Å) | | (350 Å) |
| Comparative | Compound | Compound | Compound | Compound A:Compound | Compound | Compound |
| Example 1 | HT:Compound | HT | EB | H-137:Compound RD | HB | ET:Liq |
| | HI (97:3) | (400 Å) | (50 Å) | (48.5:48.5:3) (400 Å) | (50 Å) | (40:60) |
| | (100 Å) | | | | | (350 Å) |
| Comparative | Compound | Compound | Compound | Compound B:Compound | Compound | Compound |
| Example 2 | HT:Compound | HT | EB | H-137:Compound RD | HB | ET:Liq |
| | HI (97:3) | (400 Å) | (50 Å) | (48.5:48.5:3) (400 Å) | (50 Å) | (40:60) |
| | (100 Å) | | | | | (350 Å) |

The materials used in the devices have the following structures:

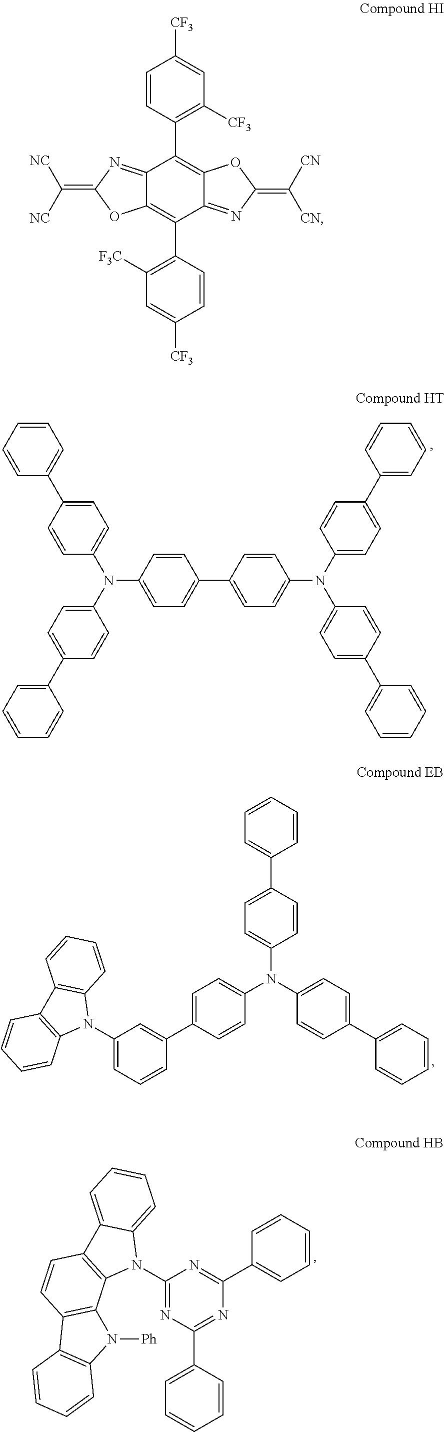 [SMILES: N#CC(C#N)=c1nc2c(-c3ccc(C(F)(F)F)cc3C(F)(F)F)c3oc(=C(C#N)C#N)nc3c(-c3ccc(C(F)(F)F)cc3C(F)(F)F)c2o1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2nc(-c3ccccc3)nc(-n3c4ccccc4c4ccc5c6ccccc6n(-c6ccccc6)c5c43)n2)cc1]

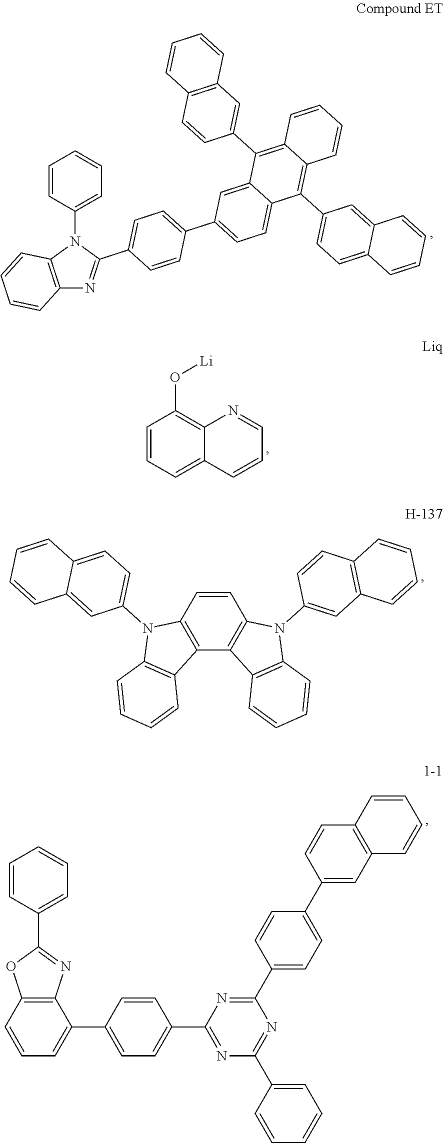

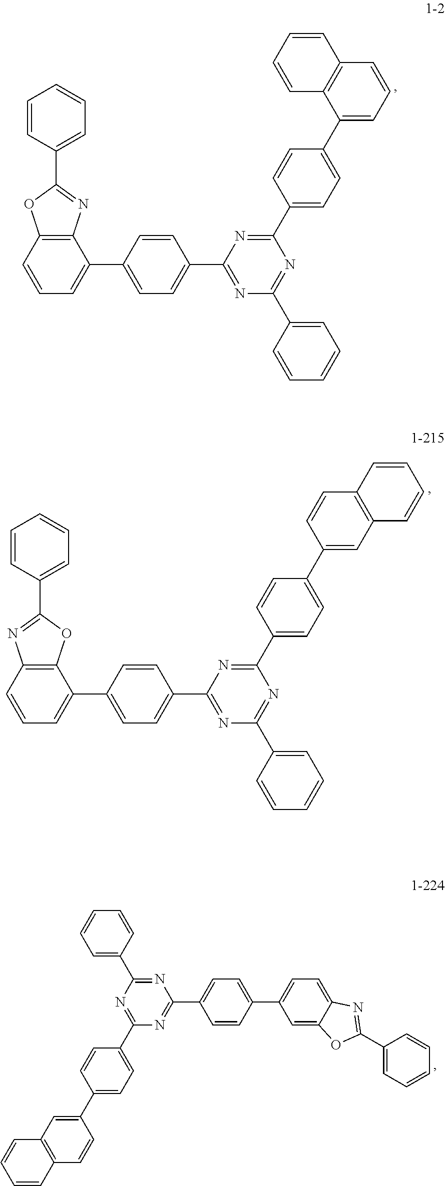 [SMILES: c1ccc(-c2nc(-c3ccc(-c4cccc5ccccc45)cc3)nc(-c3ccc(-c4cccc5oc(-c6ccccc6)nc45)cc3)n2)cc1 ; c1ccc(-c2nc(-c3ccc(-c4ccc5ccccc5c4)cc3)nc(-c3ccc(-c4cccc5nc(-c6ccccc6)oc45)cc3)n2)cc1 ; c1ccc(-c2nc(-c3ccc(-c4ccc5ccccc5c4)cc3)nc(-c3ccc(-c4ccc5nc(-c6ccccc6)oc5c4)cc3)n2)cc1]

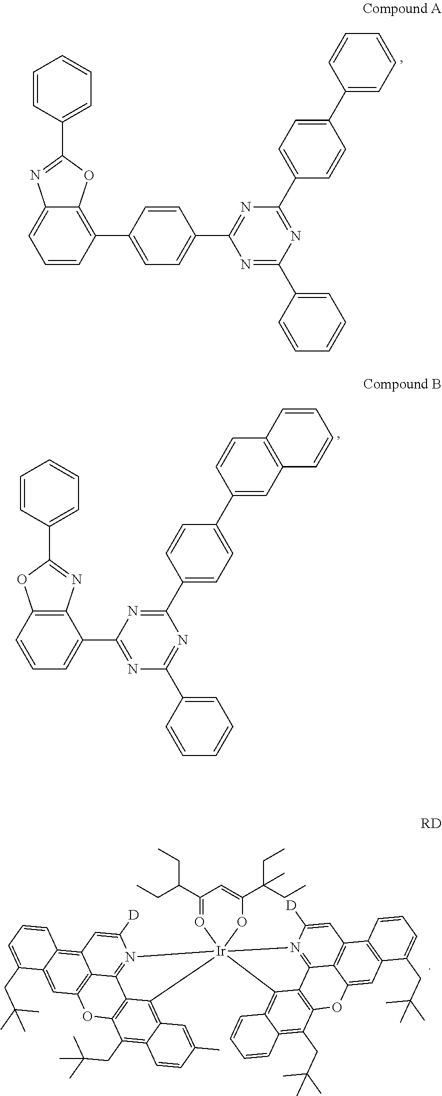

The maximum emission wavelength λₘₐₓ (nm) and power efficiency PE (lm/W) measured at a constant current of 10 mA/cm² and the device lifetime (LT97) measured at a constant current of 80 mA/cm² are listed in Table 2, where the device lifetime (LT97) refers to the time for the device's brightness to decay to 97% of its initial brightness.

TABLE 2
Device data

| Device ID | λₘₐₓ (nm) | PE [lm/W] | LT97 (h) |
|---|---|---|---|
| Example 1 | 622 | 25.7 | 118 |
| Example 2 | 623 | 25 | 82 |
| Example 3 | 622 | 25.4 | 135.5 |
| Example 4 | 622 | 25.7 | 94 |
| Comparative | 622 | 25.7 | 73 |
| Example 1 | | | |
| Comparative | 622 | 23.6 | 6 |
| Example 2 | | | |

As can be seen from the data in Table 2, the examples and comparative examples had basically the same maximum emission wavelength. Example 1 differs from Comparative Example 1 only in that the triazinyl group in Example 1 and Comparative Example 1 is connected to naphthylphenyl and biphenyl, respectively. Example 1 maintained the same high power efficiency as Comparative Example 1. In terms of lifetime, Example 1 was unexpectedly 45 hours longer, which is significantly increased by 62%, compared with Comparative Example 1. The above data indicates that the compound of the present disclosure in which the naphthylphenyl triazinyl group is connected to benzoxazole via L₁ can achieve good device performance when applied to the organic electroluminescent device.

Example 3 differs from Comparative Example 2 only in whether triazine and benzoxazole are connected via a particular group L₁. The power efficiency of Example 3 was significantly improved by 8% compared with that of Comparative Example 2, and the lifetime of Example 3 was unexpectedly increased by 2158% compared with that of Comparative Example 2. The above data indicates that the compound of the present disclosure in which the triazinyl group having a particular structure is connected to benzoxazole via a particular connection group L₁ can achieve a longer device lifetime when applied to the organic electroluminescent device.

Based on Example 1 and Example 3, Example 2 changed the connection position of the triazinyl group on benzoxazole via L₁ and Example 4 changed the connection position of naphthyl. Both Example 2 and Example 4 achieved high device efficiency and long device lifetimes. Compared with Comparative Examples 1 and 2, Examples 2 and 4 had comparative or further improved power efficiency, while significantly increased device lifetimes. The above results further indicate that the compound in which the naphthylphenyl triazinyl group is connected to benzoxazole via a particular connection group L₁ can achieve good device performance when applied to the organic electroluminescent device, proving again the unique advantages of the compound of the present disclosure.

As can be seen from the above results, when applied to the organic electroluminescent device, the compound having the structure of Formula 1 disclosed in the present disclosure can achieve good device performance, achieve high device efficiency, and especially increase the device lifetime significantly.

It should be understood that various embodiments described herein are merely embodiments and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It should be understood that various theories as to why the present disclosure works are not intended to be limitative.

What is claimed is:

1. A compound having a structure of Formula 1: 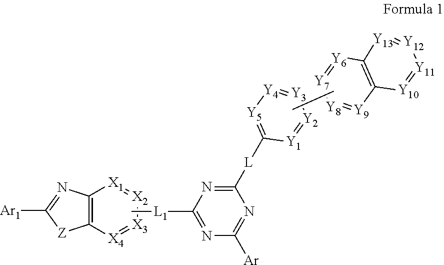
wherein
X₁ to X₄ are, at each occurrence identically or differently, selected from C, N, or CRₓ, and one of X₁ to X₄ is selected from C and joined to L₁;
Y₁ to Y₉ are, at each occurrence identically or differently, selected from C, N, or CR_{y}, one of Y₁ to Y₅ is selected from C, and one of Y₆ to Y₉ is selected from C;
Y₁₀ to Y₁₃ are, at each occurrence identically or differently, selected from N or CR_{y};
Z is, at each occurrence identically or differently, selected from O, S, or Se;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and when Rₓ is selected from substituted aryl having 6 to 30 carbon atoms, the aryl is substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar is selected from substituted or unsubstituted aryl having 6 to 30 carbon atoms, substituted or unsubstituted heteroaryl having 3 to 30 carbon atoms, or a combination thereof;
Ar₁ is selected from substituted or unsubstituted aryl having 6 to 30 carbon atoms;
L is selected from a single bond, substituted or unsubstituted arylene having 6 to 30 carbon atoms, substituted or unsubstituted heteroarylene having 3 to 30 carbon atoms, or a combination thereof;
L₁ is selected from substituted or unsubstituted arylene having 6 to 30 carbon atoms, substituted or unsubstituted heteroarylene having 3 to 30 carbon atoms, or a combination thereof;
adjacent substituents R_{y} in Y₁ to Y₅ can be optionally joined to form a ring; and
adjacent substituents R_{y} in Y₆ to Y₁₃ can be optionally joined to form a ring.

2. The compound of claim 1, wherein the compound has a structure represented by any one of Formula 1-1 to Formula 1-4: 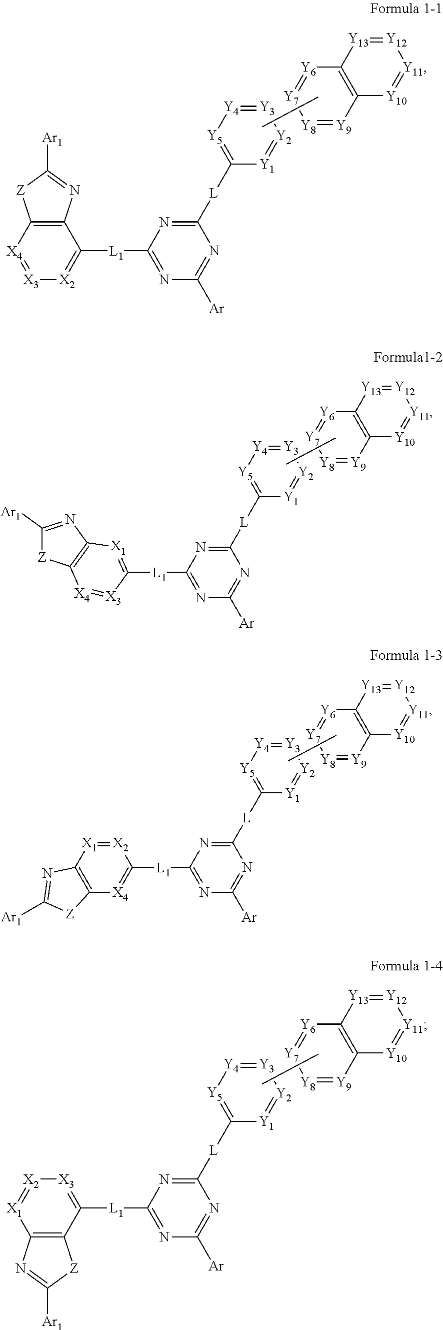
wherein
X₁ to X₄ are, at each occurrence identically or differently, selected from N or CRₓ;
Y₁ to Y₉ are, at each occurrence identically or differently, selected from C, N, or CR_{y}, one of Y₁ to Y₅ is selected from C, and one of Y₆ to Y₉ is selected from C;
Y₁₀ to Y₁₃ are, at each occurrence identically or differently, selected from N or CR_{y};
Z is, at each occurrence identically or differently, selected from O, S, or Se;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, a cyano group, an isocyano group, a hydroxyl group, a sulfanyl group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, a cyano group, an isocyano group, a hydroxyl group, a sulfanyl group, and combinations thereof; and when Rₓ is selected from substituted aryl having 6 to 30 carbon atoms, the aryl is substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar is selected from substituted or unsubstituted aryl having 6 to 30 carbon atoms, substituted or unsubstituted heteroaryl having 3 to 30 carbon atoms, or a combination thereof;
Ar₁ is selected from substituted or unsubstituted aryl having 6 to 30 carbon atoms;
L is selected from a single bond, substituted or unsubstituted arylene having 6 to 30 carbon atoms, substituted or unsubstituted heteroarylene having 3 to 30 carbon atoms, or a combination thereof;
L₁ is selected from substituted or unsubstituted arylene having 6 to 30 carbon atoms, substituted or unsubstituted heteroarylene having 3 to 30 carbon atoms, or a combination thereof;
adjacent substituents R_{y} in Y₁ to Y₅ can be optionally joined to form a ring; and
adjacent substituents R_{y} in Y₆ to Y₁₃ can be optionally joined to form a ring.

3. The compound of claim 1, wherein R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, a cyano group, an isocyano group, a hydroxyl group, a sulfanyl group, and combinations thereof; and
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 20 carbon atoms, a cyano group, an isocyano group, a hydroxyl group, a sulfanyl group, and combinations thereof;
preferably, R_{y} is, at each occurrence identically or differently, selected from the group consisting of: hydrogen, deuterium, fluorine, methyl, phenyl, deuterated naphthyl, deuterated phenyl, pyridyl, vinyl, naphthyl, biphenyl, phenanthryl, triphenylenyl, dibenzofuryl, dibenzothienyl, cyano, adamantyl, t-butyl, trifluoromethyl, carbazolyl, 9,9-dimethylfluorenyl, and combinations thereof; and
Rₓ is, at each occurrence identically or differently, selected from the group consisting of: hydrogen, deuterium, fluorine, methyl, phenyl, deuterated naphthyl, deuterated phenyl, naphthyl, biphenyl, phenanthryl, triphenylenyl, cyano, adamantyl, t-butyl, trifluoromethyl, 9,9-dimethylfluorenyl, and combinations thereof.

4. The compound of claim 1, wherein Z is selected from O or S; preferably, Z is O.

5. The compound of claim 1, wherein X₁ to X₄ are, at each occurrence identically or differently, selected from C or CRₓ, Y₁ to Y₉ are, at each occurrence identically or differently, selected from C or CR_{y}, and Y₁₀ to Y₁₃ are, at each occurrence identically or differently, selected from CR_{y}.

6. The compound of claim 1, wherein Ar is selected from substituted or unsubstituted aryl having 6 to 18 carbon atoms, substituted or unsubstituted heteroaryl having 3 to 12 carbon atoms, or a combination thereof; and
Ar₁ is selected from substituted or unsubstituted aryl having 6 to 18 carbon atoms;
preferably, Ar is selected from the group consisting of: substituted or unsubstituted naphthyl, substituted or unsubstituted phenyl, substituted or unsubstituted naphthylphenyl, substituted or unsubstituted terphenyl, substituted or unsubstituted biphenyl, substituted or unsubstituted phenanthryl, substituted or unsubstituted triphenylenyl, substituted or unsubstituted pyrimidinyl, substituted or unsubstituted pyridyl, substituted or unsubstituted dibenzofuryl, substituted or unsubstituted dibenzothienyl, substituted or unsubstituted 9,9-dimethylfluorenyl, and combinations thereof; and
Ar₁ is selected from the group consisting of: substituted or unsubstituted naphthyl, substituted or unsubstituted phenyl, substituted or unsubstituted naphthylphenyl, substituted or unsubstituted terphenyl, substituted or unsubstituted biphenyl, substituted or unsubstituted phenanthryl, substituted or unsubstituted triphenylenyl, substituted or unsubstituted 9,9-dimethylfluorenyl, and combinations thereof.

7. The compound of claim 1, wherein L is selected from a single bond or substituted or unsubstituted arylene having 6 to 30 carbon atoms; and
L₁ is selected from substituted or unsubstituted arylene having 6 to 30 carbon atoms;
preferably, L is selected from the group consisting of: a single bond, substituted or unsubstituted phenylene, substituted or unsubstituted biphenylylene, substituted or unsubstituted naphthylene, substituted or unsubstituted phenanthrylene, substituted or unsubstituted triphenylenylene, and combinations thereof; and
L₁ is selected from the group consisting of: substituted or unsubstituted phenylene, substituted or unsubstituted biphenylylene, substituted or unsubstituted naphthylene, substituted or unsubstituted phenanthrylene, substituted or unsubstituted triphenylenylene, and combinations thereof;
more preferably, L is selected from a single bond, and L₁ is selected from substituted or unsubstituted phenylene or substituted or unsubstituted naphthylene.

8. The compound of claim 1, wherein one of Y₁ to Y₃ is selected from C and joined to Y₆ or Y₇;
preferably, Y₃ is selected from C and joined to Y₆ or Y₇.

9. The compound of claim 1, wherein the compound is selected from the group consisting of the following compounds: 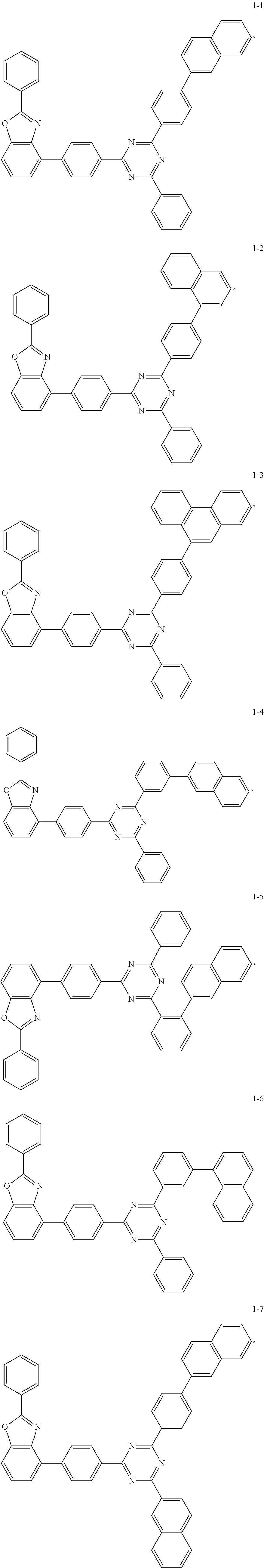 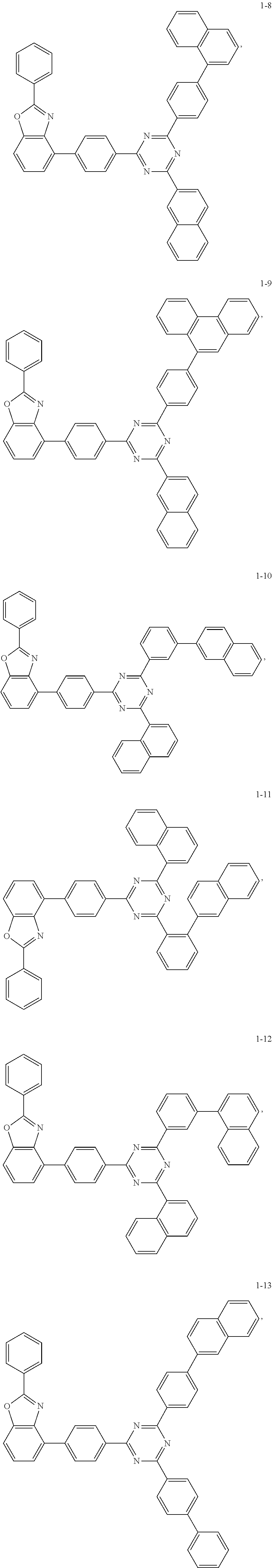 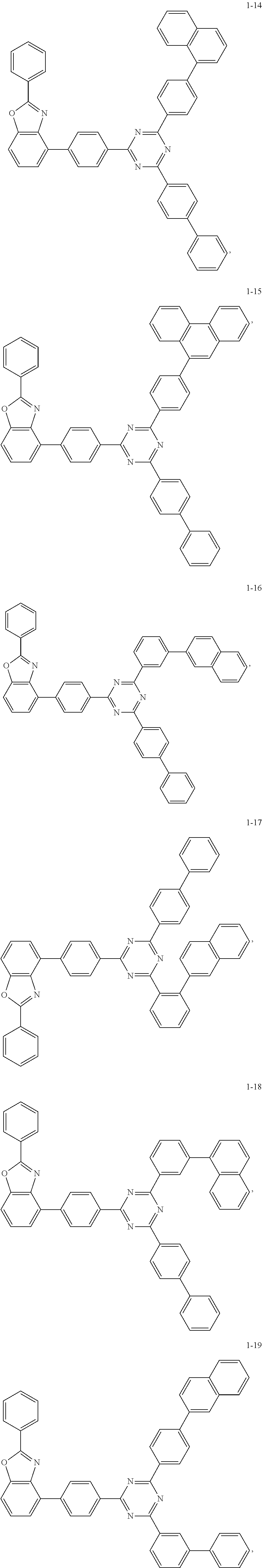 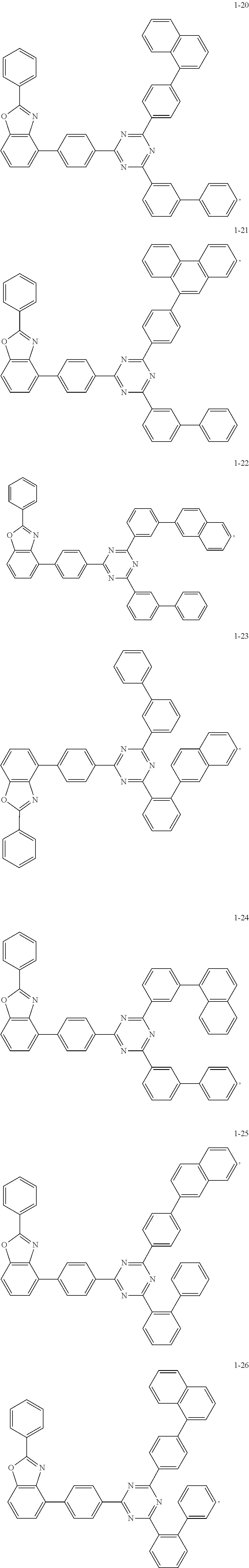 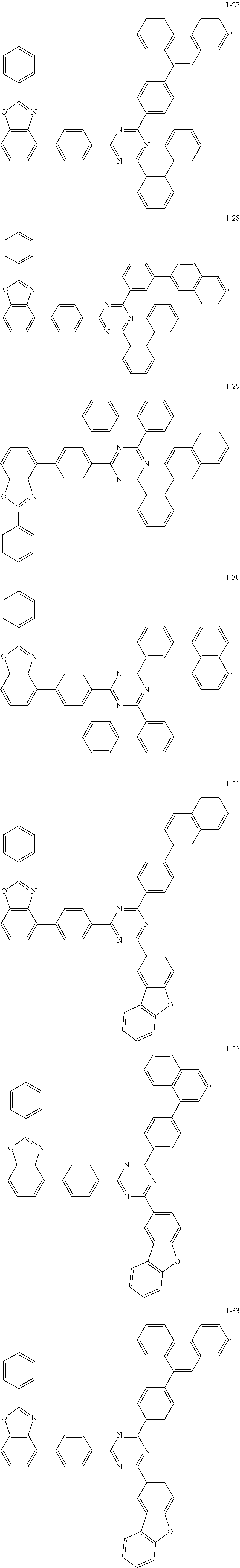 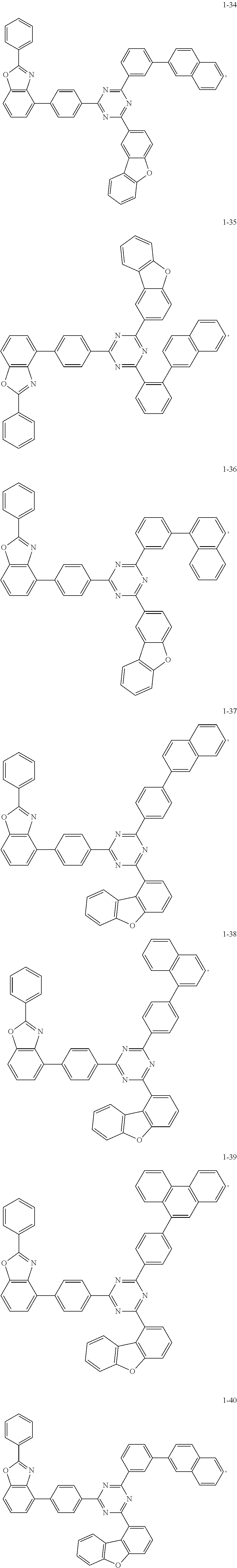 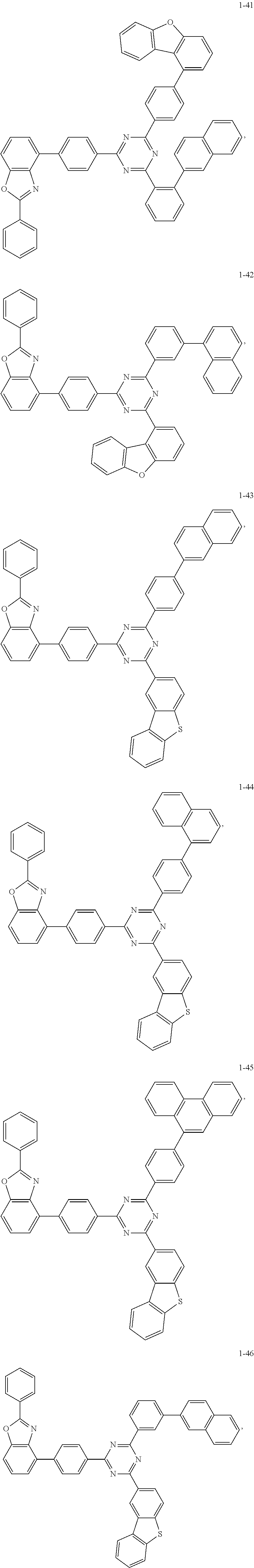 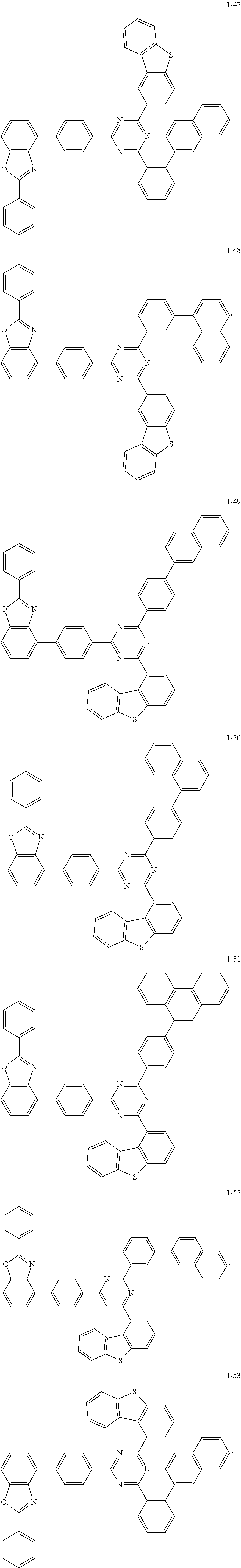 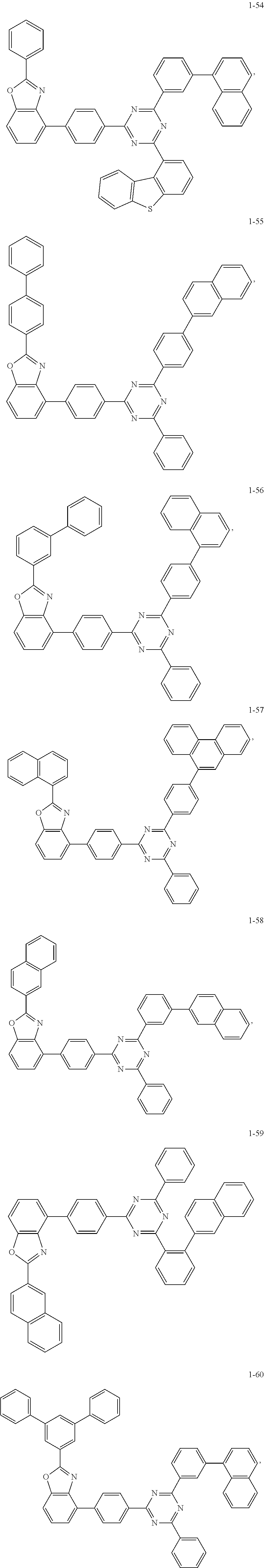 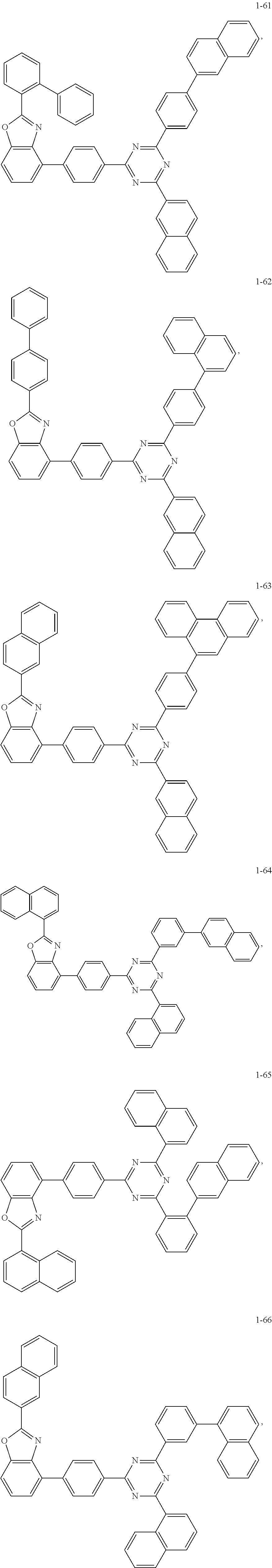 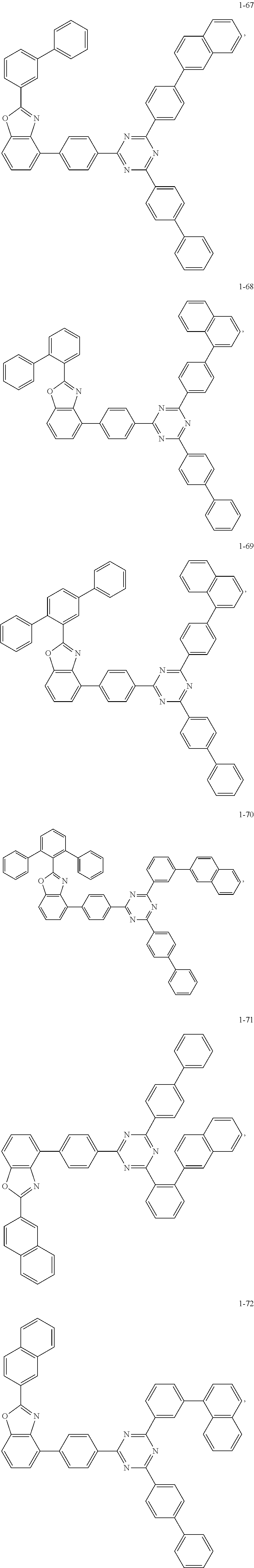 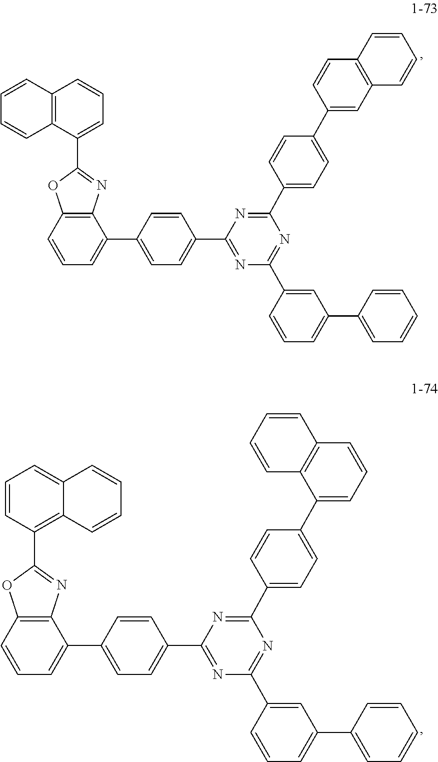 [SMILES: c1ccc(-c2cccc(-c3nc(-c4ccc(-c5ccc6ccccc6c5)cc4)nc(-c4ccc(-c5cccc6oc(-c7cccc8ccccc78)nc56)cc4)n3)c2)cc1 ; c1ccc(-c2cccc(-c3nc(-c4ccc(-c5cccc6ccccc56)cc4)nc(-c4ccc(-c5cccc6oc(-c7cccc8ccccc78)nc56)cc4)n3)c2)cc1] 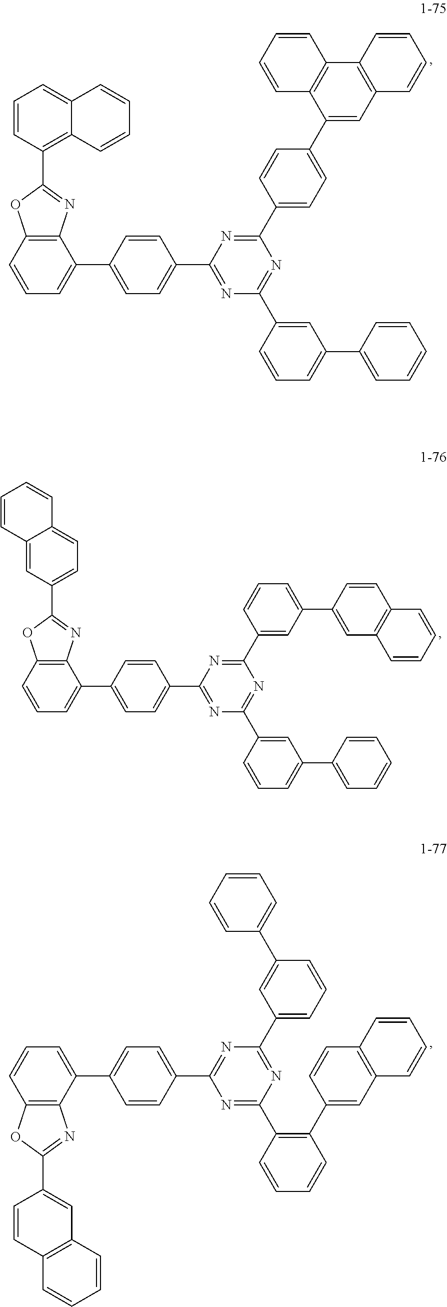 [SMILES: c1ccc(-c2cccc(-c3nc(-c4ccc(-c5cc6ccccc6c6ccccc56)cc4)nc(-c4ccc(-c5cccc6oc(-c7cccc8ccccc78)nc56)cc4)n3)c2)cc1 ; c1ccc(-c2cccc(-c3nc(-c4ccc(-c5cccc6oc(-c7ccc8ccccc8c7)nc56)cc4)nc(-c4cccc(-c5ccc6ccccc6c5)c4)n3)c2)cc1 ; c1ccc(-c2cccc(-c3nc(-c4ccc(-c5cccc6oc(-c7ccc8ccccc8c7)nc56)cc4)nc(-c4ccccc4-c4ccc5ccccc5c4)n3)c2)cc1] 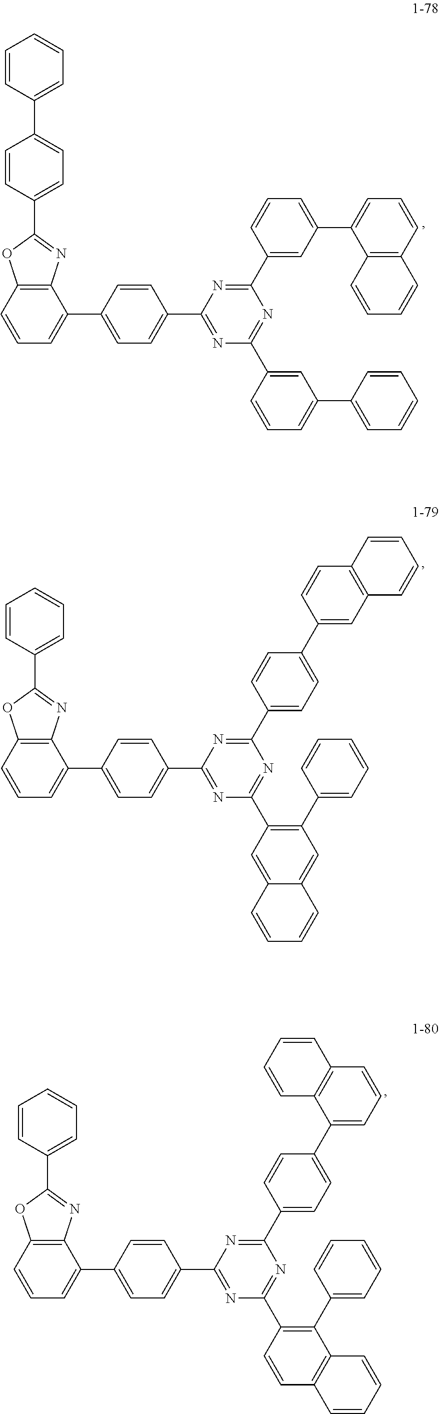 [SMILES: c1ccc(-c2ccc(-c3nc4c(-c5ccc(-c6nc(-c7cccc(-c8ccccc8)c7)nc(-c7cccc(-c8cccc9ccccc89)c7)n6)cc5)cccc4o3)cc2)cc1 ; c1ccc(-c2nc3c(-c4ccc(-c5nc(-c6ccc(-c7ccc8ccccc8c7)cc6)nc(-c6cc7ccccc7cc6-c6ccccc6)n5)cc4)cccc3o2)cc1 ; c1ccc(-c2nc3c(-c4ccc(-c5nc(-c6ccc(-c7cccc8ccccc78)cc6)nc(-c6ccc7ccccc7c6-c6ccccc6)n5)cc4)cccc3o2)cc1] 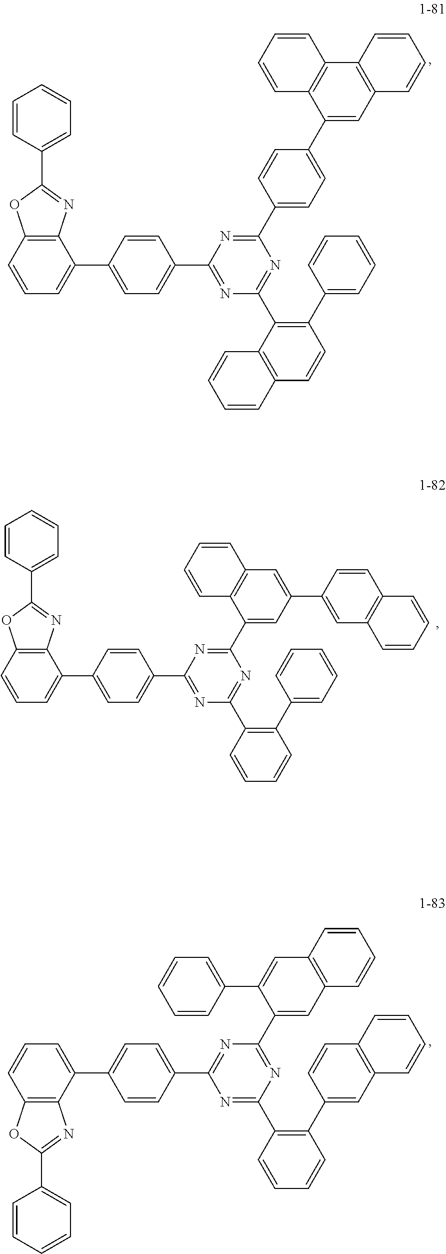 [SMILES: c1ccc(-c2nc3c(-c4ccc(-c5nc(-c6ccc(-c7cc8ccccc8c8ccccc78)cc6)nc(-c6c(-c7ccccc7)ccc7ccccc67)n5)cc4)cccc3o2)cc1 ; c1ccc(-c2nc3c(-c4ccc(-c5nc(-c6ccccc6-c6ccccc6)nc(-c6cc(-c7ccc8ccccc8c7)cc7ccccc67)n5)cc4)cccc3o2)cc1 ; c1ccc(-c2nc3c(-c4ccc(-c5nc(-c6ccccc6-c6ccc7ccccc7c6)nc(-c6cc7ccccc7cc6-c6ccccc6)n5)cc4)cccc3o2)cc1] 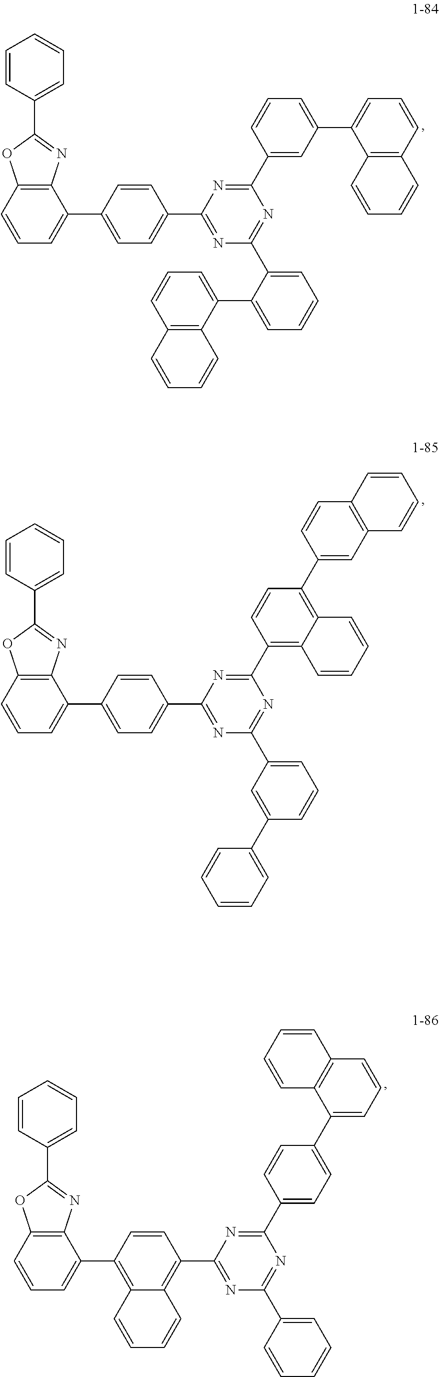 [SMILES: c1ccc(-c2nc3c(-c4ccc(-c5nc(-c6cccc(-c7cccc8ccccc78)c6)nc(-c6ccccc6-c6cccc7ccccc67)n5)cc4)cccc3o2)cc1 ; c1ccc(-c2cccc(-c3nc(-c4ccc(-c5cccc6oc(-c7ccccc7)nc56)cc4)nc(-c4ccc(-c5ccc6ccccc6c5)c5ccccc45)n3)c2)cc1 ; c1ccc(-c2nc(-c3ccc(-c4cccc5ccccc45)cc3)nc(-c3ccc(-c4cccc5oc(-c6ccccc6)nc45)c4ccccc34)n2)cc1] 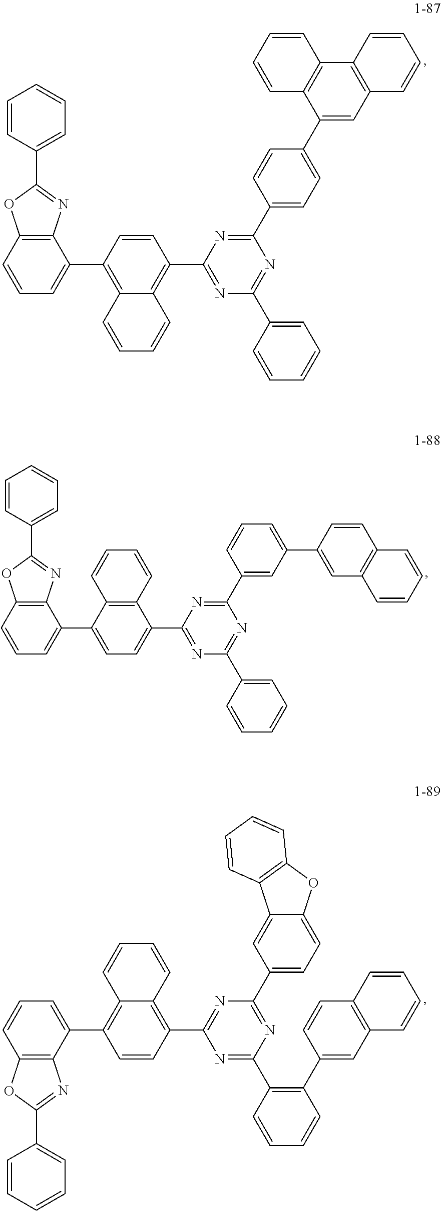 [SMILES: c1ccc(-c2nc(-c3ccc(-c4cc5ccccc5c5ccccc45)cc3)nc(-c3ccc(-c4cccc5oc(-c6ccccc6)nc45)c4ccccc34)n2)cc1 ; c1ccc(-c2nc(-c3cccc(-c4ccc5ccccc5c4)c3)nc(-c3ccc(-c4cccc5oc(-c6ccccc6)nc45)c4ccccc34)n2)cc1 ; c1ccc(-c2nc3c(-c4ccc(-c5nc(-c6ccc7oc8ccccc8c7c6)nc(-c6ccccc6-c6ccc7ccccc7c6)n5)c5ccccc45)cccc3o2)cc1] 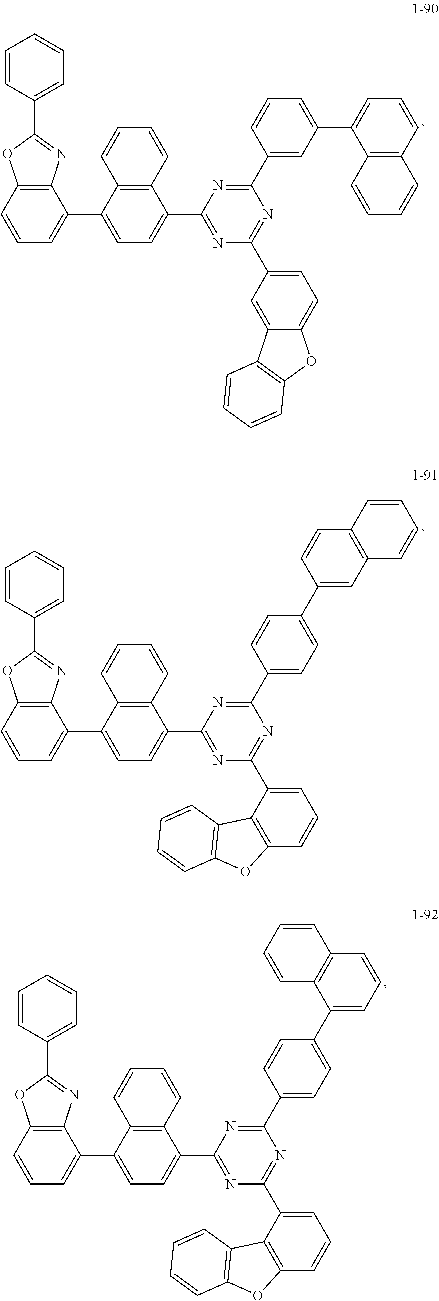 [SMILES: c1ccc(-c2nc3c(-c4ccc(-c5nc(-c6cccc(-c7cccc8ccccc78)c6)nc(-c6ccc7oc8ccccc8c7c6)n5)c5ccccc45)cccc3o2)cc1 ; c1ccc(-c2nc3c(-c4ccc(-c5nc(-c6ccc(-c7ccc8ccccc8c7)cc6)nc(-c6cccc7oc8ccccc8c67)n5)c5ccccc45)cccc3o2)cc1 ; c1ccc(-c2nc3c(-c4ccc(-c5nc(-c6ccc(-c7cccc8ccccc78)cc6)nc(-c6cccc7oc8ccccc8c67)n5)c5ccccc45)cccc3o2)cc1] 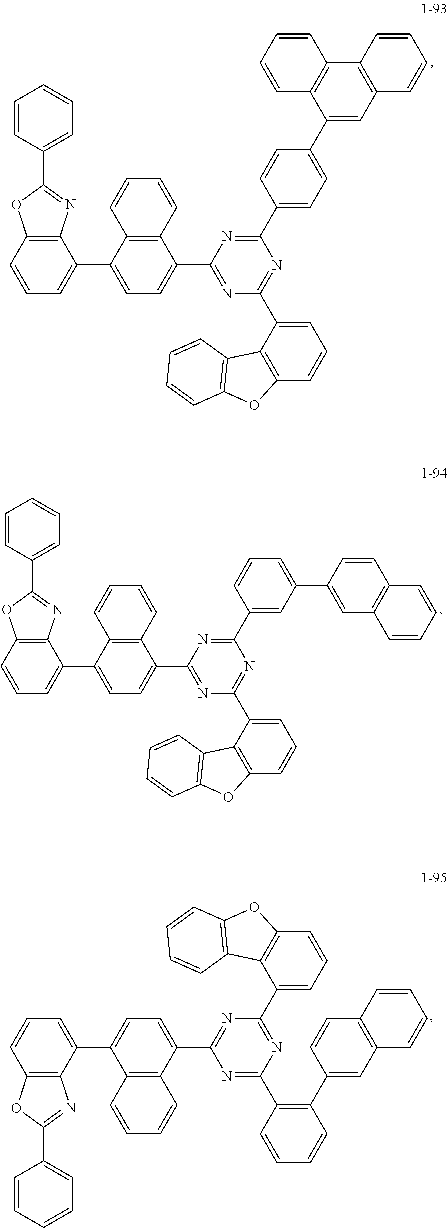 [SMILES: c1ccc(-c2nc3c(-c4ccc(-c5nc(-c6ccc(-c7cc8ccccc8c8ccccc78)cc6)nc(-c6cccc7oc8ccccc8c67)n5)c5ccccc45)cccc3o2)cc1 ; c1ccc(-c2nc3c(-c4ccc(-c5nc(-c6cccc(-c7ccc8ccccc8c7)c6)nc(-c6cccc7oc8ccccc8c67)n5)c5ccccc45)cccc3o2)cc1 ; c1ccc(-c2nc3c(-c4ccc(-c5nc(-c6ccccc6-c6ccc7ccccc7c6)nc(-c6cccc7oc8ccccc8c67)n5)c5ccccc45)cccc3o2)cc1] 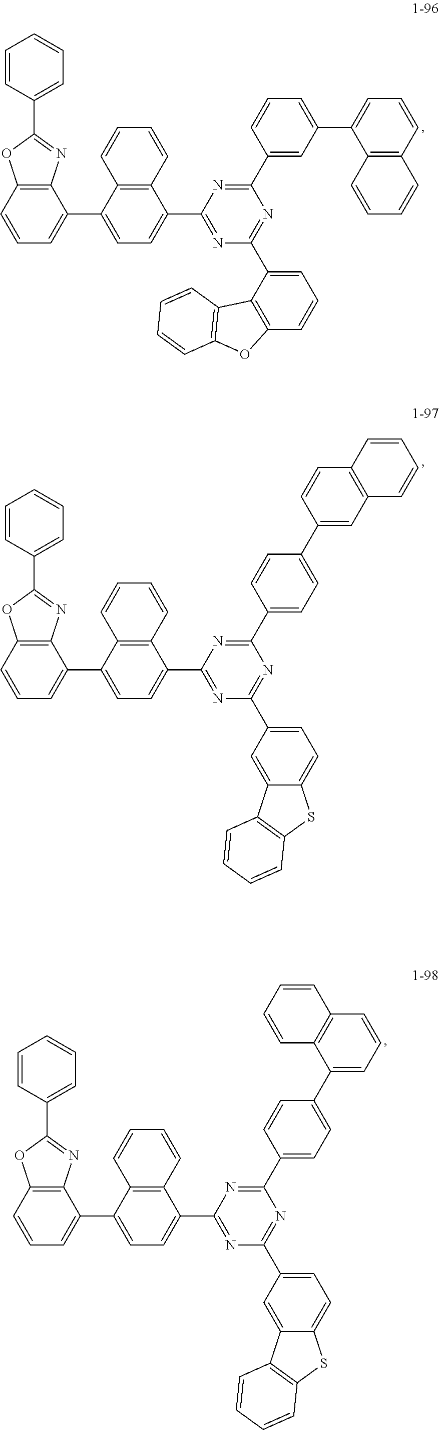 [SMILES: c1ccc(-c2nc3c(-c4ccc(-c5nc(-c6cccc(-c7cccc8ccccc78)c6)nc(-c6cccc7oc8ccccc8c67)n5)c5ccccc45)cccc3o2)cc1 ; c1ccc(-c2nc3c(-c4ccc(-c5nc(-c6ccc(-c7ccc8ccccc8c7)cc6)nc(-c6ccc7sc8ccccc8c7c6)n5)c5ccccc45)cccc3o2)cc1 ; c1ccc(-c2nc3c(-c4ccc(-c5nc(-c6ccc(-c7cccc8ccccc78)cc6)nc(-c6ccc7sc8ccccc8c7c6)n5)c5ccccc45)cccc3o2)cc1] 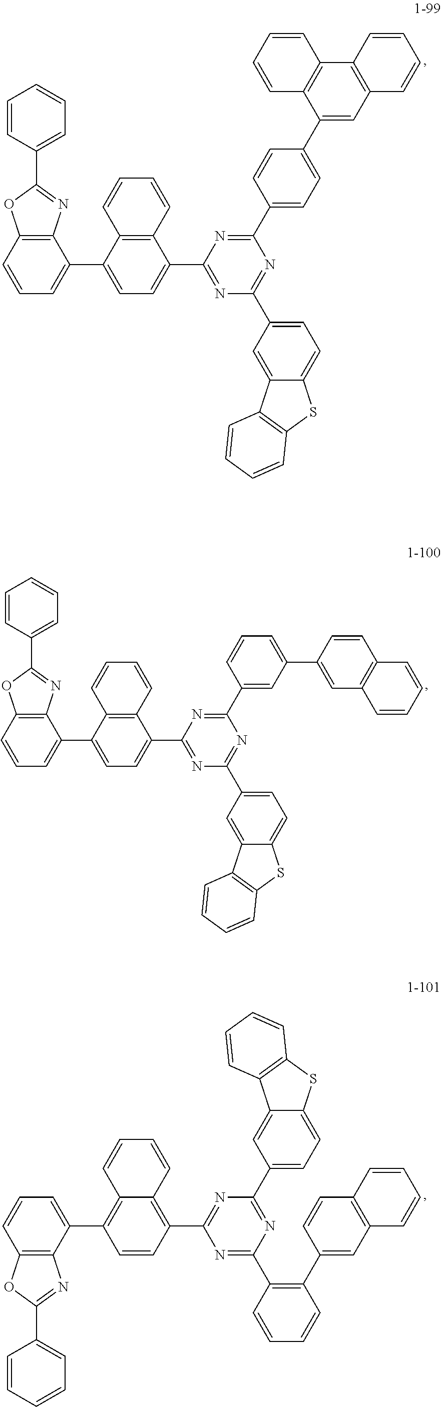 [SMILES: c1ccc(-c2nc3c(-c4ccc(-c5nc(-c6ccc(-c7cc8ccccc8c8ccccc78)cc6)nc(-c6ccc7sc8ccccc8c7c6)n5)c5ccccc45)cccc3o2)cc1 ; c1ccc(-c2nc3c(-c4ccc(-c5nc(-c6cccc(-c7ccc8ccccc8c7)c6)nc(-c6ccc7sc8ccccc8c7c6)n5)c5ccccc45)cccc3o2)cc1 ; c1ccc(-c2nc3c(-c4ccc(-c5nc(-c6ccc7sc8ccccc8c7c6)nc(-c6ccccc6-c6ccc7ccccc7c6)n5)c5ccccc45)cccc3o2)cc1] 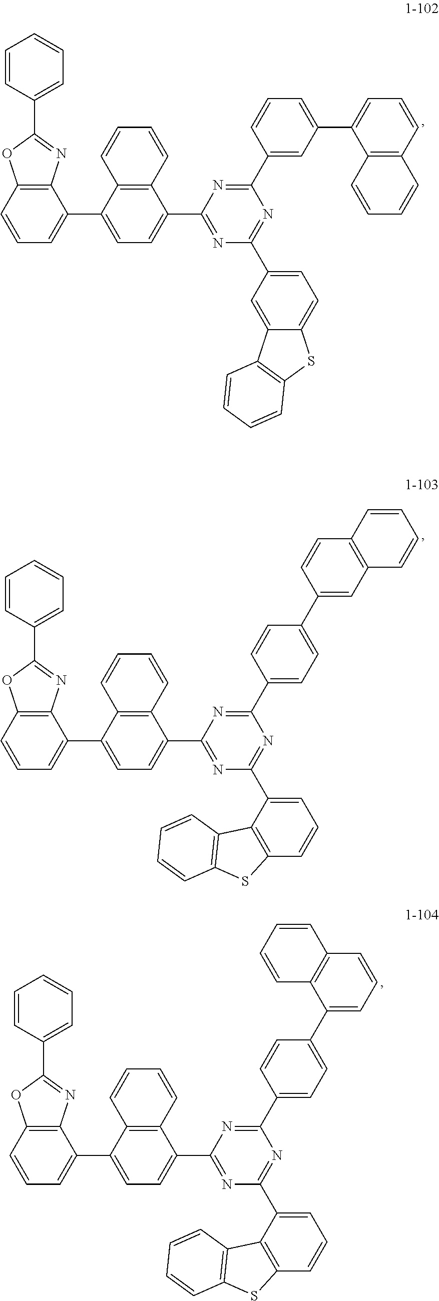 [SMILES: c1ccc(-c2nc3c(-c4ccc(-c5nc(-c6cccc(-c7cccc8ccccc78)c6)nc(-c6ccc7sc8ccccc8c7c6)n5)c5ccccc45)cccc3o2)cc1 ; c1ccc(-c2nc3c(-c4ccc(-c5nc(-c6ccc(-c7ccc8ccccc8c7)cc6)nc(-c6cccc7sc8ccccc8c67)n5)c5ccccc45)cccc3o2)cc1 ; c1ccc(-c2nc3c(-c4ccc(-c5nc(-c6ccc(-c7cccc8ccccc78)cc6)nc(-c6cccc7sc8ccccc8c67)n5)c5ccccc45)cccc3o2)cc1] 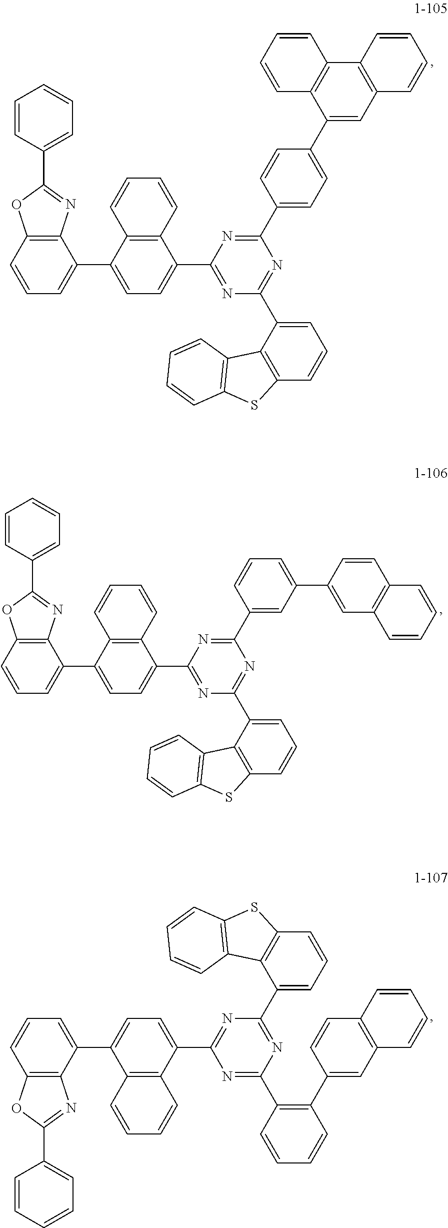 [SMILES: c1ccc(-c2nc3c(-c4ccc(-c5nc(-c6ccc(-c7cc8ccccc8c8ccccc78)cc6)nc(-c6cccc7sc8ccccc8c67)n5)c5ccccc45)cccc3o2)cc1 ; c1ccc(-c2nc3c(-c4ccc(-c5nc(-c6cccc(-c7ccc8ccccc8c7)c6)nc(-c6cccc7sc8ccccc8c67)n5)c5ccccc45)cccc3o2)cc1 ; c1ccc(-c2nc3c(-c4ccc(-c5nc(-c6ccccc6-c6ccc7ccccc7c6)nc(-c6cccc7sc8ccccc8c67)n5)c5ccccc45)cccc3o2)cc1] 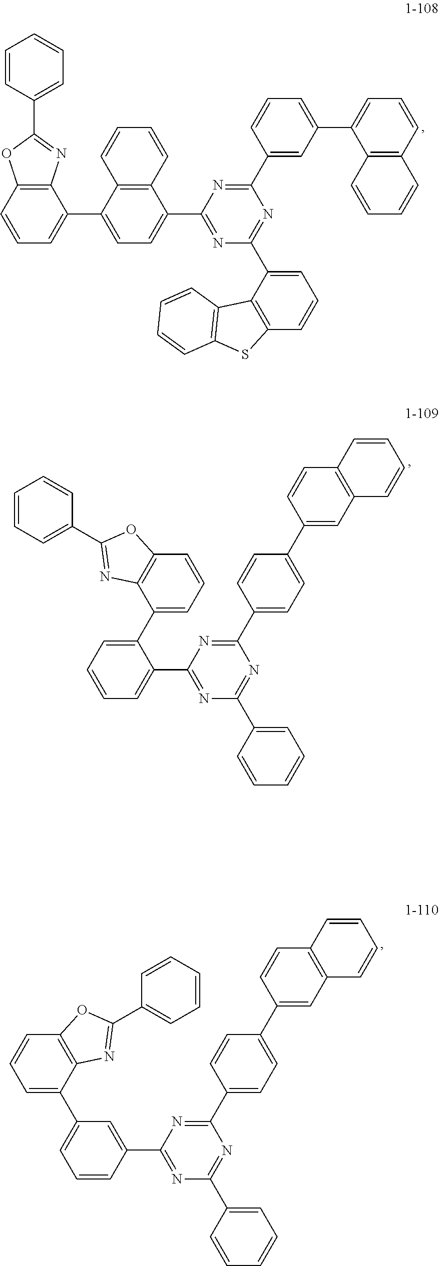 [SMILES: c1ccc(-c2nc3c(-c4ccc(-c5nc(-c6cccc(-c7cccc8ccccc78)c6)nc(-c6cccc7sc8ccccc8c67)n5)c5ccccc45)cccc3o2)cc1 ; c1ccc(-c2nc(-c3ccc(-c4ccc5ccccc5c4)cc3)nc(-c3ccccc3-c3cccc4oc(-c5ccccc5)nc34)n2)cc1 ; c1ccc(-c2nc(-c3ccc(-c4ccc5ccccc5c4)cc3)nc(-c3cccc(-c4cccc5oc(-c6ccccc6)nc45)c3)n2)cc1] 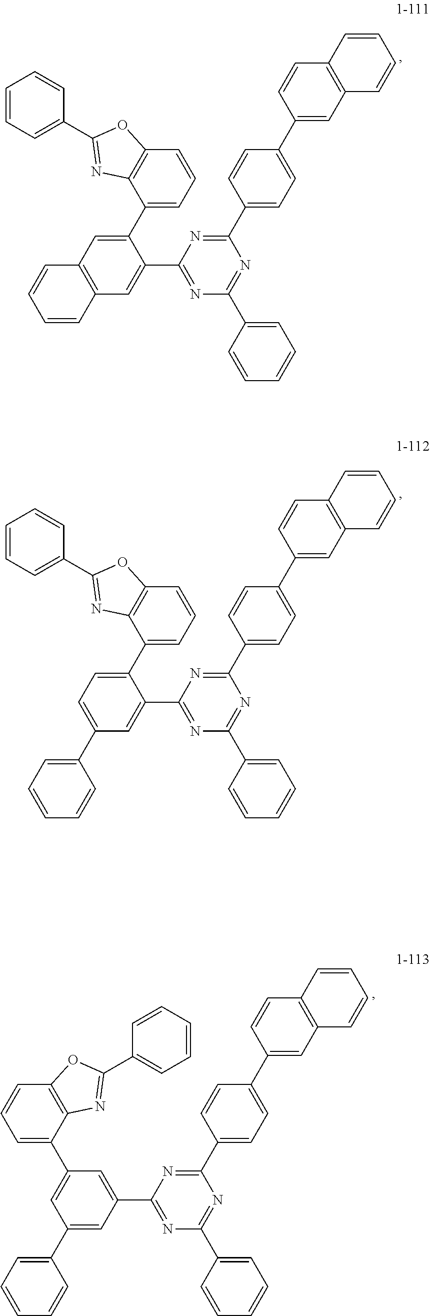 [SMILES: c1ccc(-c2nc(-c3ccc(-c4ccc5ccccc5c4)cc3)nc(-c3cc4ccccc4cc3-c3cccc4oc(-c5ccccc5)nc34)n2)cc1 ; c1ccc(-c2ccc(-c3cccc4oc(-c5ccccc5)nc34)c(-c3nc(-c4ccccc4)nc(-c4ccc(-c5ccc6ccccc6c5)cc4)n3)c2)cc1 ; c1ccc(-c2cc(-c3nc(-c4ccccc4)nc(-c4ccc(-c5ccc6ccccc6c5)cc4)n3)cc(-c3cccc4oc(-c5ccccc5)nc34)c2)cc1] 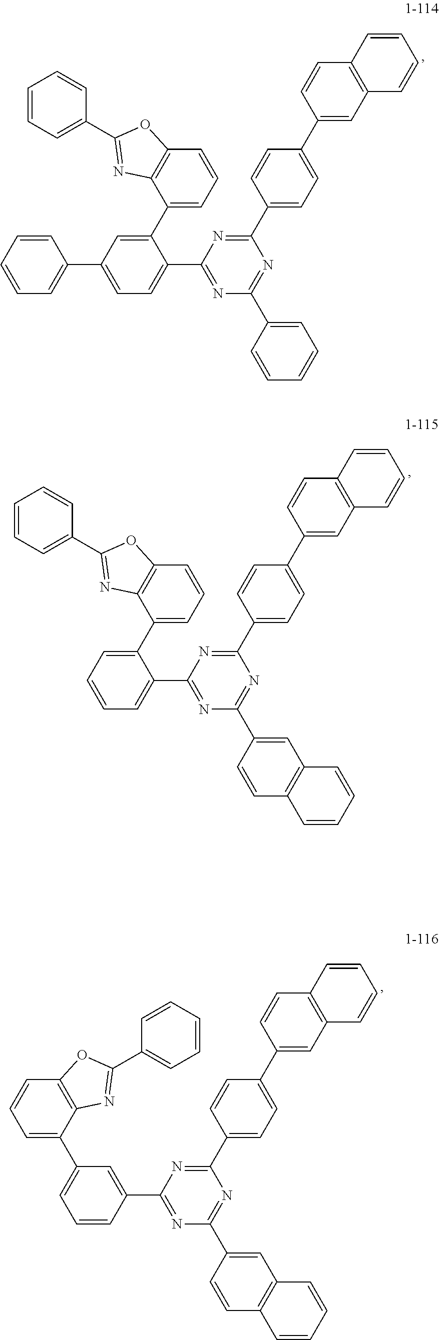 [SMILES: c1ccc(-c2ccc(-c3nc(-c4ccccc4)nc(-c4ccc(-c5ccc6ccccc6c5)cc4)n3)c(-c3cccc4oc(-c5ccccc5)nc34)c2)cc1 ; c1ccc(-c2nc3c(-c4ccccc4-c4nc(-c5ccc(-c6ccc7ccccc7c6)cc5)nc(-c5ccc6ccccc6c5)n4)cccc3o2)cc1 ; c1ccc(-c2nc3c(-c4cccc(-c5nc(-c6ccc(-c7ccc8ccccc8c7)cc6)nc(-c6ccc7ccccc7c6)n5)c4)cccc3o2)cc1] 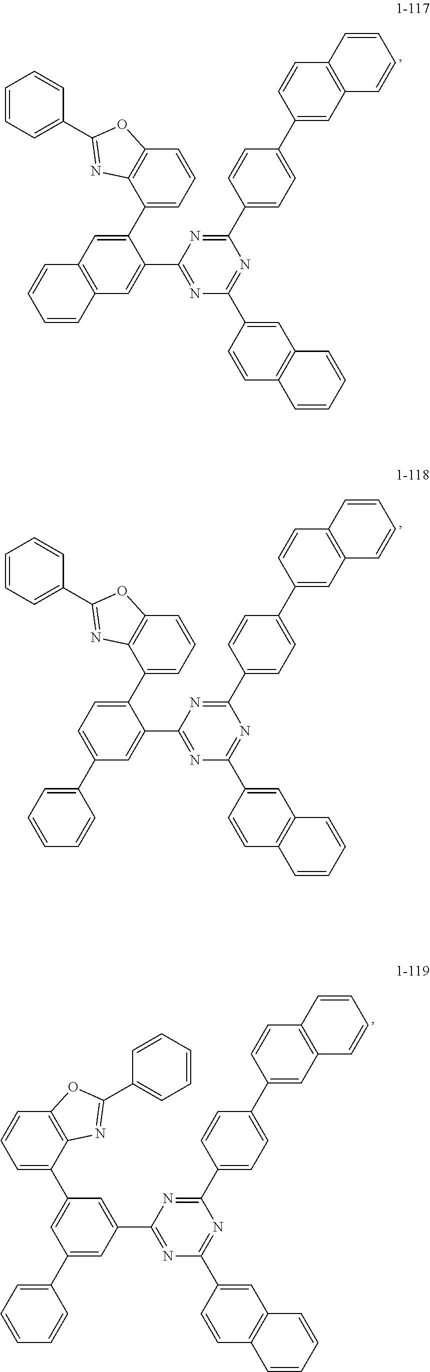 [SMILES: c1ccc(-c2nc3c(-c4cc5ccccc5cc4-c4nc(-c5ccc(-c6ccc7ccccc7c6)cc5)nc(-c5ccc6ccccc6c5)n4)cccc3o2)cc1 ; c1ccc(-c2ccc(-c3cccc4oc(-c5ccccc5)nc34)c(-c3nc(-c4ccc(-c5ccc6ccccc6c5)cc4)nc(-c4ccc5ccccc5c4)n3)c2)cc1 ; c1ccc(-c2cc(-c3nc(-c4ccc(-c5ccc6ccccc6c5)cc4)nc(-c4ccc5ccccc5c4)n3)cc(-c3cccc4oc(-c5ccccc5)nc34)c2)cc1] 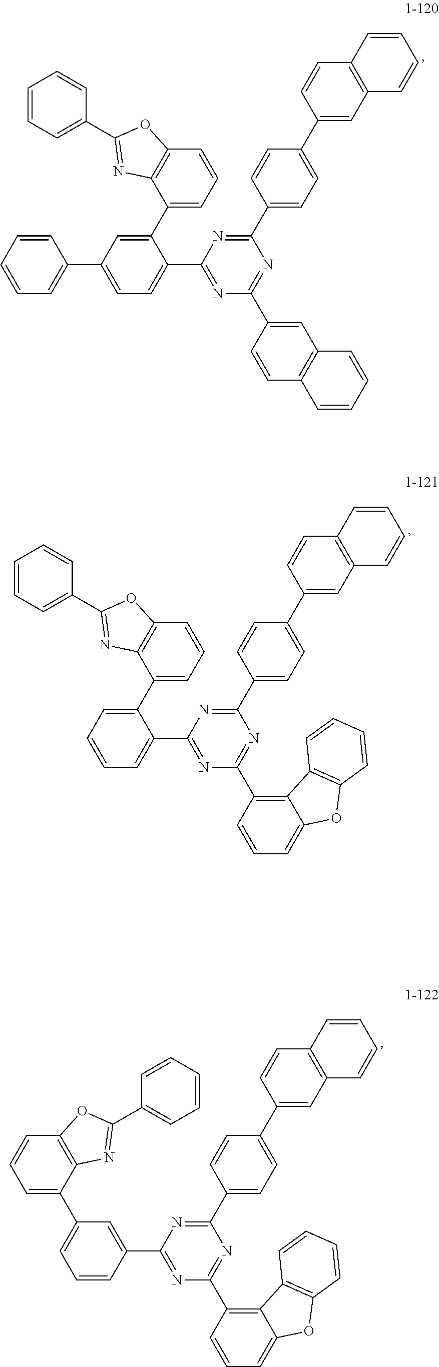 [SMILES: c1ccc(-c2ccc(-c3nc(-c4ccc(-c5ccc6ccccc6c5)cc4)nc(-c4ccc5ccccc5c4)n3)c(-c3cccc4oc(-c5ccccc5)nc34)c2)cc1 ; c1ccc(-c2nc3c(-c4ccccc4-c4nc(-c5ccc(-c6ccc7ccccc7c6)cc5)nc(-c5cccc6oc7ccccc7c56)n4)cccc3o2)cc1 ; c1ccc(-c2nc3c(-c4cccc(-c5nc(-c6ccc(-c7ccc8ccccc8c7)cc6)nc(-c6cccc7oc8ccccc8c67)n5)c4)cccc3o2)cc1] 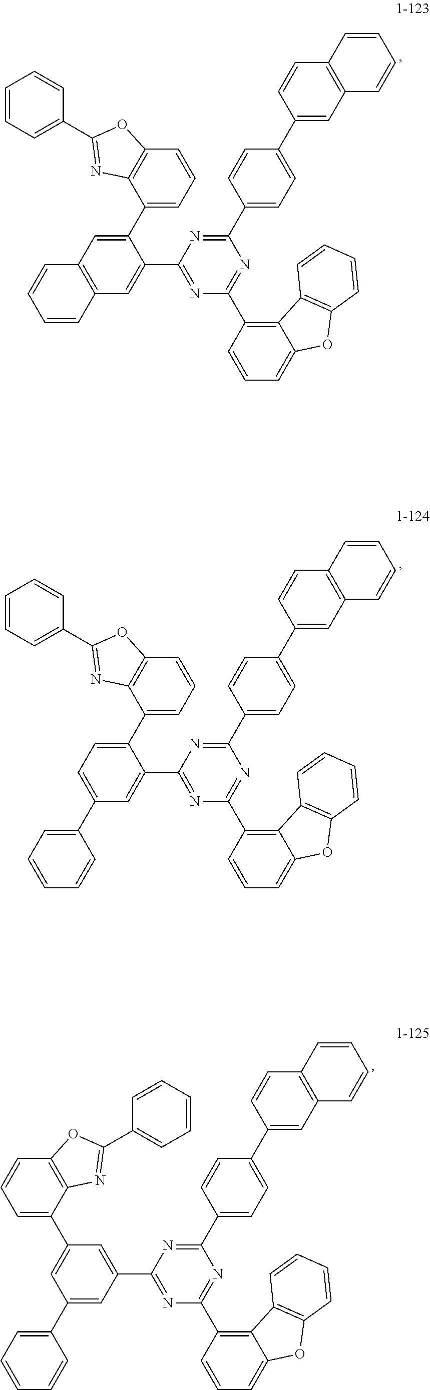 [SMILES: c1ccc(-c2nc3c(-c4cc5ccccc5cc4-c4nc(-c5ccc(-c6ccc7ccccc7c6)cc5)nc(-c5cccc6oc7ccccc7c56)n4)cccc3o2)cc1 ; c1ccc(-c2ccc(-c3cccc4oc(-c5ccccc5)nc34)c(-c3nc(-c4ccc(-c5ccc6ccccc6c5)cc4)nc(-c4cccc5oc6ccccc6c45)n3)c2)cc1 ; c1ccc(-c2cc(-c3nc(-c4ccc(-c5ccc6ccccc6c5)cc4)nc(-c4cccc5oc6ccccc6c45)n3)cc(-c3cccc4oc(-c5ccccc5)nc34)c2)cc1] 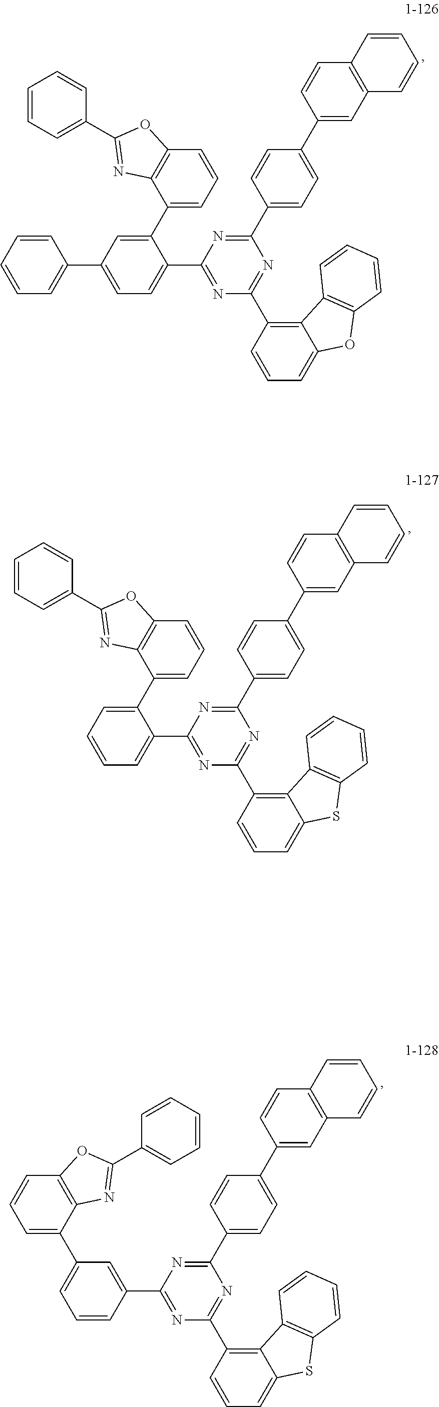 [SMILES: c1ccc(-c2ccc(-c3nc(-c4ccc(-c5ccc6ccccc6c5)cc4)nc(-c4cccc5oc6ccccc6c45)n3)c(-c3cccc4oc(-c5ccccc5)nc34)c2)cc1 ; c1ccc(-c2nc3c(-c4ccccc4-c4nc(-c5ccc(-c6ccc7ccccc7c6)cc5)nc(-c5cccc6sc7ccccc7c56)n4)cccc3o2)cc1 ; c1ccc(-c2nc3c(-c4cccc(-c5nc(-c6ccc(-c7ccc8ccccc8c7)cc6)nc(-c6cccc7sc8ccccc8c67)n5)c4)cccc3o2)cc1] 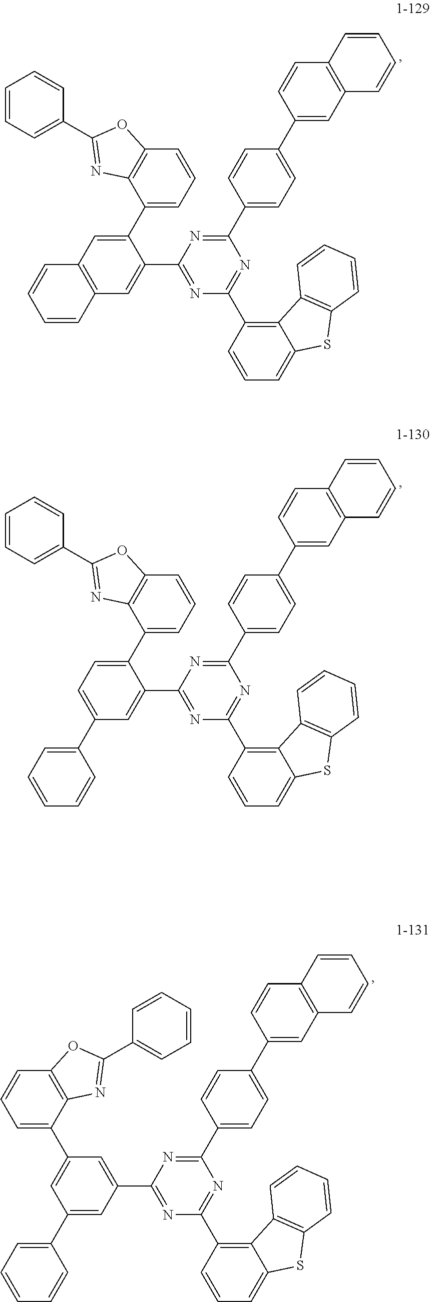 [SMILES: c1ccc(-c2nc3c(-c4cc5ccccc5cc4-c4nc(-c5ccc(-c6ccc7ccccc7c6)cc5)nc(-c5cccc6sc7ccccc7c56)n4)cccc3o2)cc1 ; c1ccc(-c2ccc(-c3cccc4oc(-c5ccccc5)nc34)c(-c3nc(-c4ccc(-c5ccc6ccccc6c5)cc4)nc(-c4cccc5sc6ccccc6c45)n3)c2)cc1 ; c1ccc(-c2cc(-c3nc(-c4ccc(-c5ccc6ccccc6c5)cc4)nc(-c4cccc5sc6ccccc6c45)n3)cc(-c3cccc4oc(-c5ccccc5)nc34)c2)cc1] 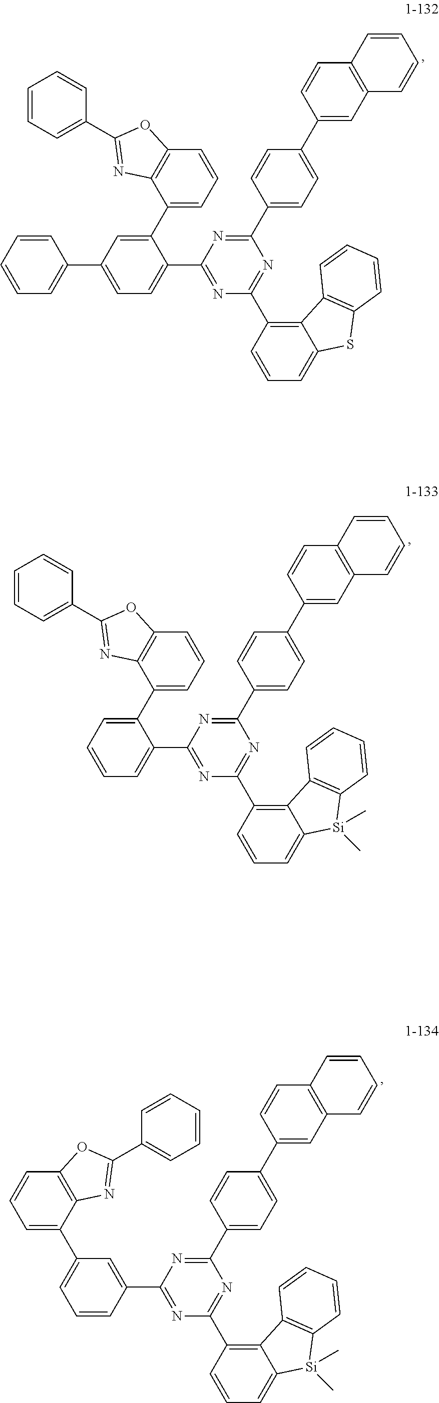 [SMILES: c1ccc(-c2ccc(-c3nc(-c4ccc(-c5ccc6ccccc6c5)cc4)nc(-c4cccc5sc6ccccc6c45)n3)c(-c3cccc4oc(-c5ccccc5)nc34)c2)cc1 ; C[Si]1(C)c2ccccc2-c2c(-c3nc(-c4ccc(-c5ccc6ccccc6c5)cc4)nc(-c4ccccc4-c4cccc5oc(-c6ccccc6)nc45)n3)cccc21 ; C[Si]1(C)c2ccccc2-c2c(-c3nc(-c4ccc(-c5ccc6ccccc6c5)cc4)nc(-c4cccc(-c5cccc6oc(-c7ccccc7)nc56)c4)n3)cccc21] 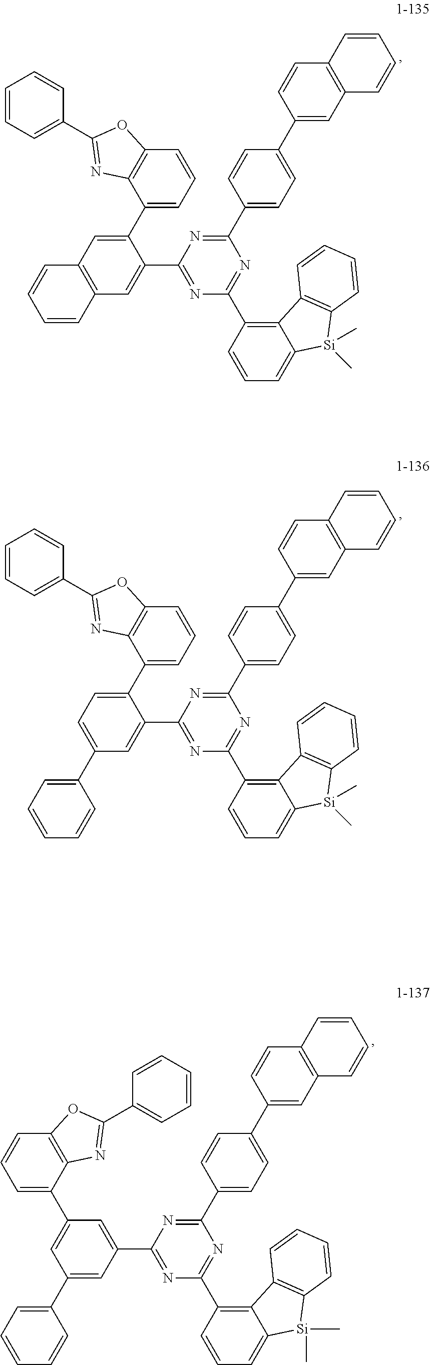 [SMILES: C[Si]1(C)c2ccccc2-c2c(-c3nc(-c4ccc(-c5ccc6ccccc6c5)cc4)nc(-c4cc5ccccc5cc4-c4cccc5oc(-c6ccccc6)nc45)n3)cccc21 ; C[Si]1(C)c2ccccc2-c2c(-c3nc(-c4ccc(-c5ccc6ccccc6c5)cc4)nc(-c4cc(-c5ccccc5)ccc4-c4cccc5oc(-c6ccccc6)nc45)n3)cccc21 ; C[Si]1(C)c2ccccc2-c2c(-c3nc(-c4ccc(-c5ccc6ccccc6c5)cc4)nc(-c4cc(-c5ccccc5)cc(-c5cccc6oc(-c7ccccc7)nc56)c4)n3)cccc21] 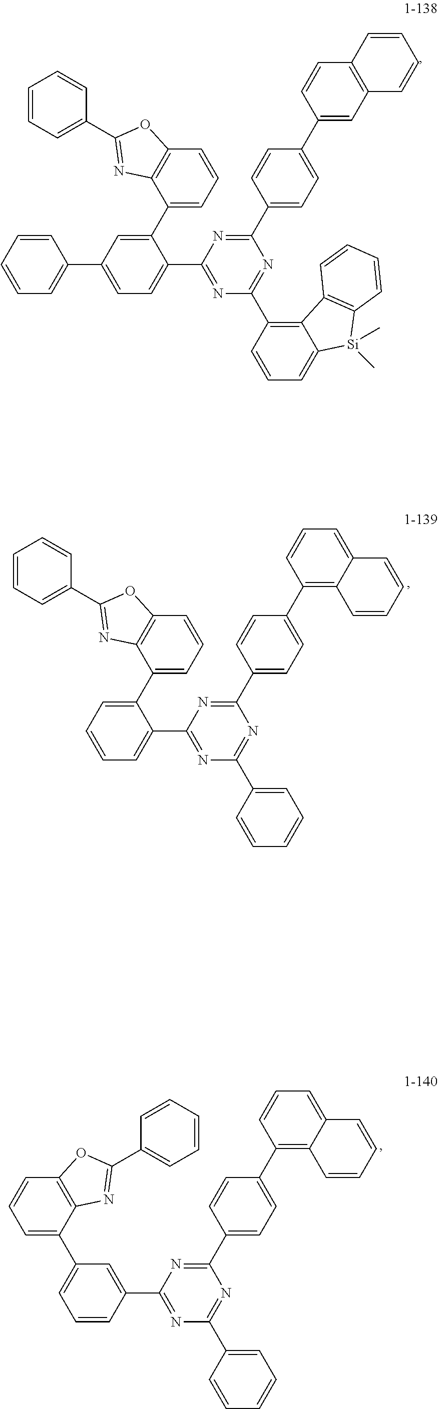 [SMILES: C[Si]1(C)c2ccccc2-c2c(-c3nc(-c4ccc(-c5ccc6ccccc6c5)cc4)nc(-c4ccc(-c5ccccc5)cc4-c4cccc5oc(-c6ccccc6)nc45)n3)cccc21 ; c1ccc(-c2nc(-c3ccc(-c4cccc5ccccc45)cc3)nc(-c3ccccc3-c3cccc4oc(-c5ccccc5)nc34)n2)cc1 ; c1ccc(-c2nc(-c3ccc(-c4cccc5ccccc45)cc3)nc(-c3cccc(-c4cccc5oc(-c6ccccc6)nc45)c3)n2)cc1] 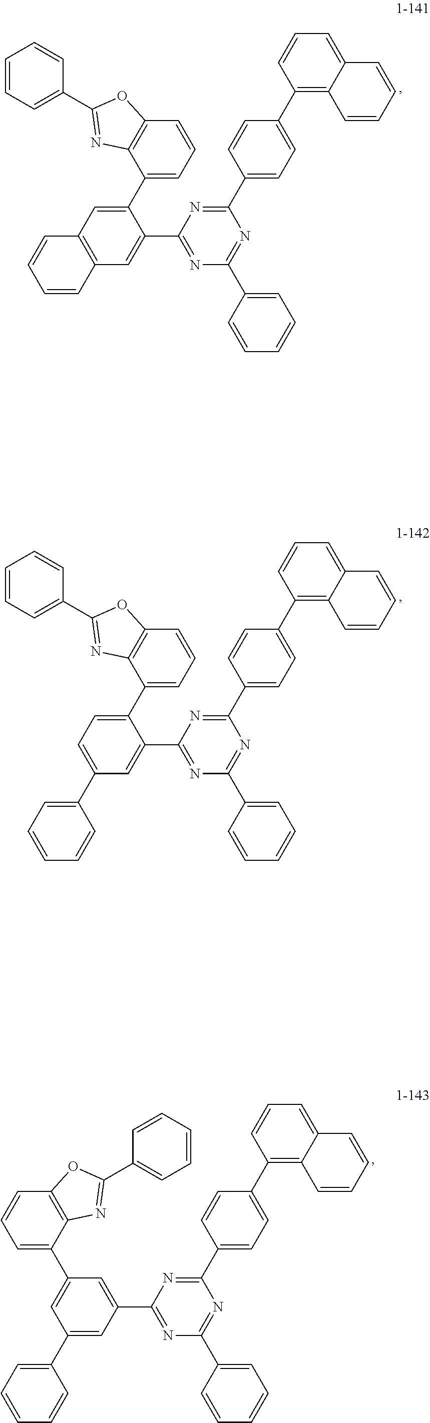 [SMILES: c1ccc(-c2nc(-c3ccc(-c4cccc5ccccc45)cc3)nc(-c3cc4ccccc4cc3-c3cccc4oc(-c5ccccc5)nc34)n2)cc1 ; c1ccc(-c2ccc(-c3cccc4oc(-c5ccccc5)nc34)c(-c3nc(-c4ccccc4)nc(-c4ccc(-c5cccc6ccccc56)cc4)n3)c2)cc1 ; c1ccc(-c2cc(-c3nc(-c4ccccc4)nc(-c4ccc(-c5cccc6ccccc56)cc4)n3)cc(-c3cccc4oc(-c5ccccc5)nc34)c2)cc1] 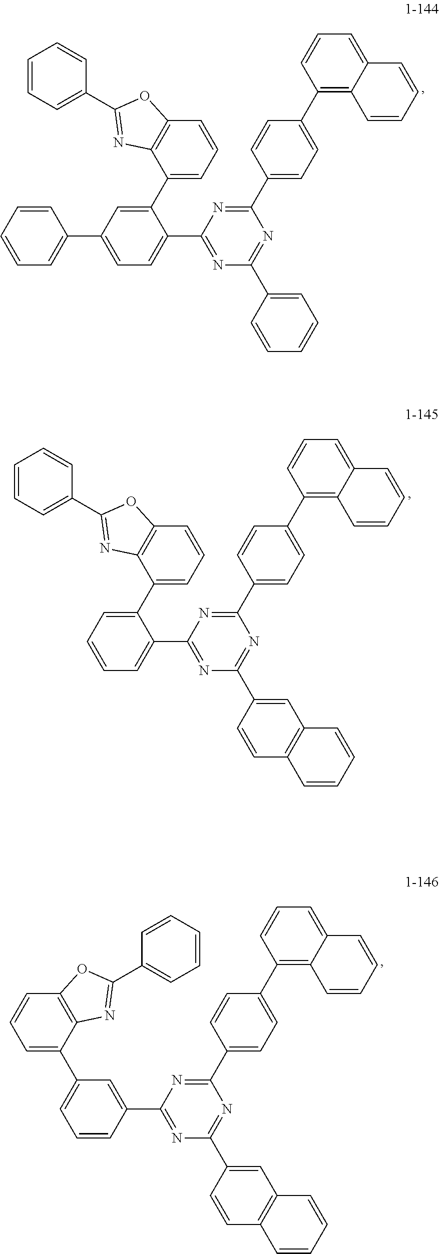 [SMILES: c1ccc(-c2ccc(-c3nc(-c4ccccc4)nc(-c4ccc(-c5cccc6ccccc56)cc4)n3)c(-c3cccc4oc(-c5ccccc5)nc34)c2)cc1 ; c1ccc(-c2nc3c(-c4ccccc4-c4nc(-c5ccc(-c6cccc7ccccc67)cc5)nc(-c5ccc6ccccc6c5)n4)cccc3o2)cc1 ; c1ccc(-c2nc3c(-c4cccc(-c5nc(-c6ccc(-c7cccc8ccccc78)cc6)nc(-c6ccc7ccccc7c6)n5)c4)cccc3o2)cc1] 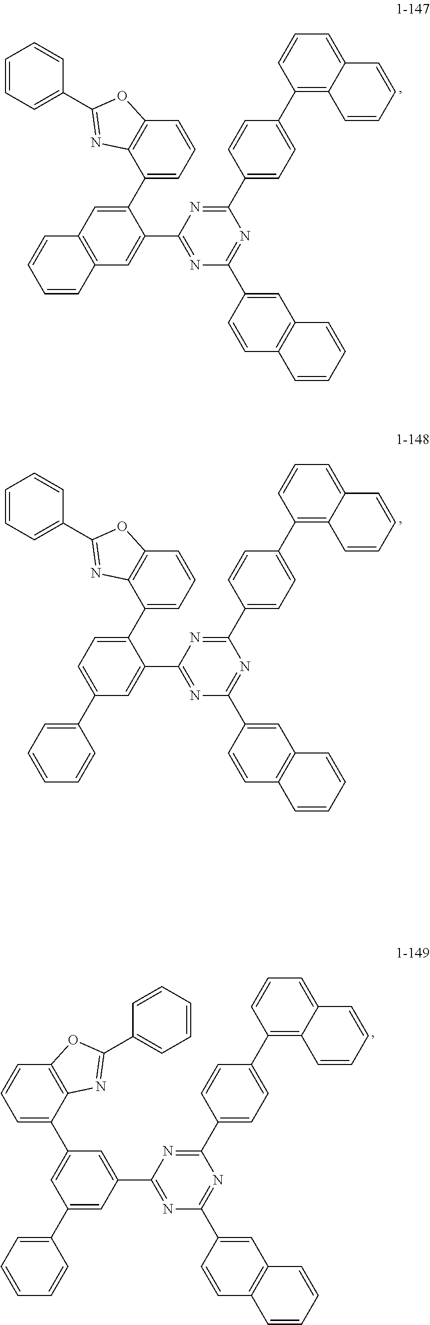 [SMILES: c1ccc(-c2nc3c(-c4cc5ccccc5cc4-c4nc(-c5ccc(-c6cccc7ccccc67)cc5)nc(-c5ccc6ccccc6c5)n4)cccc3o2)cc1 ; c1ccc(-c2ccc(-c3cccc4oc(-c5ccccc5)nc34)c(-c3nc(-c4ccc(-c5cccc6ccccc56)cc4)nc(-c4ccc5ccccc5c4)n3)c2)cc1 ; c1ccc(-c2cc(-c3nc(-c4ccc(-c5cccc6ccccc56)cc4)nc(-c4ccc5ccccc5c4)n3)cc(-c3cccc4oc(-c5ccccc5)nc34)c2)cc1] 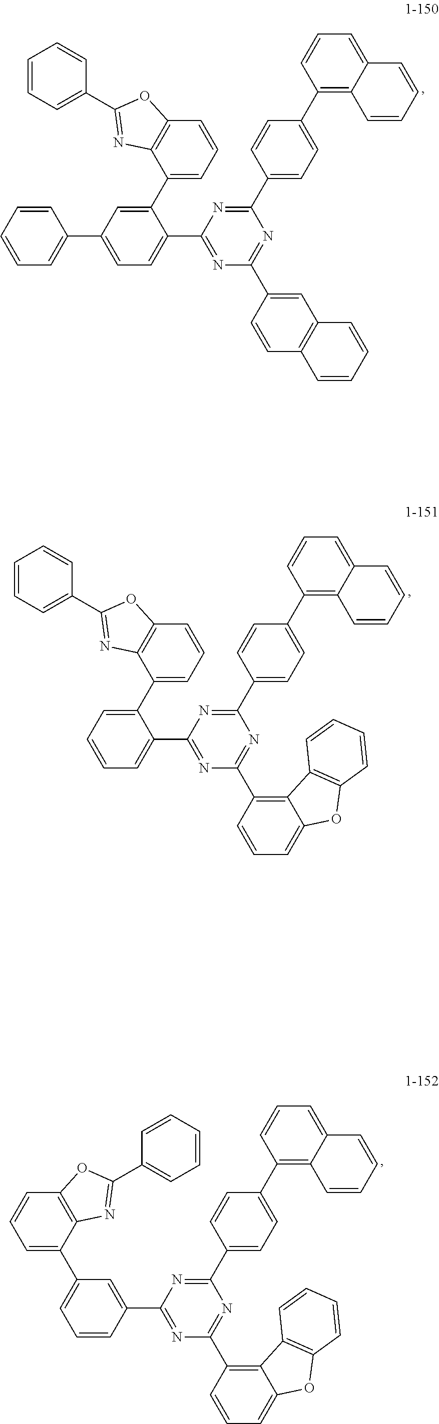 [SMILES: c1ccc(-c2ccc(-c3nc(-c4ccc(-c5cccc6ccccc56)cc4)nc(-c4ccc5ccccc5c4)n3)c(-c3cccc4oc(-c5ccccc5)nc34)c2)cc1 ; c1ccc(-c2nc3c(-c4ccccc4-c4nc(-c5ccc(-c6cccc7ccccc67)cc5)nc(-c5cccc6oc7ccccc7c56)n4)cccc3o2)cc1 ; c1ccc(-c2nc3c(-c4cccc(-c5nc(-c6ccc(-c7cccc8ccccc78)cc6)nc(-c6cccc7oc8ccccc8c67)n5)c4)cccc3o2)cc1] 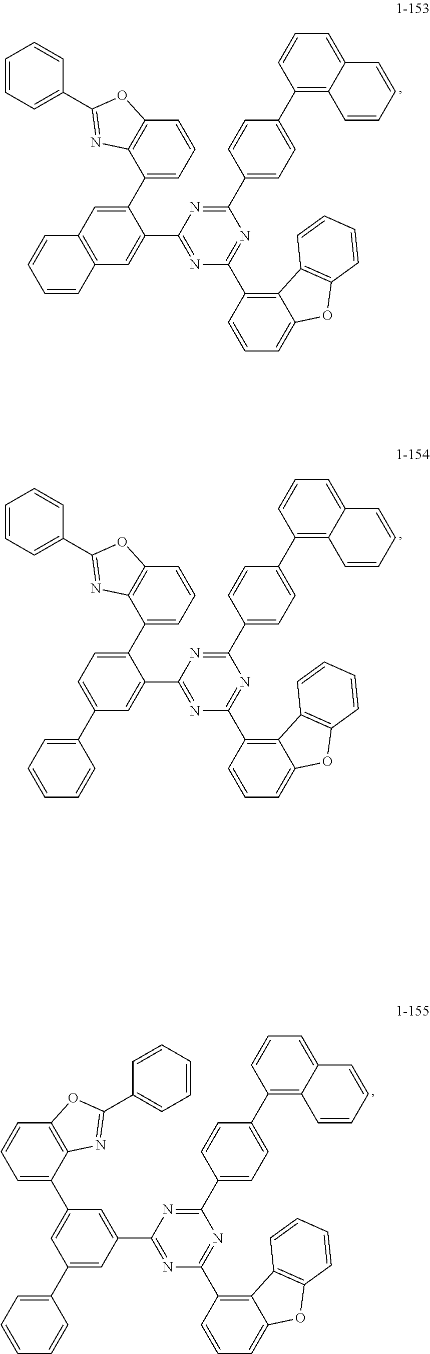 [SMILES: c1ccc(-c2nc3c(-c4cc5ccccc5cc4-c4nc(-c5ccc(-c6cccc7ccccc67)cc5)nc(-c5cccc6oc7ccccc7c56)n4)cccc3o2)cc1 ; c1ccc(-c2ccc(-c3cccc4oc(-c5ccccc5)nc34)c(-c3nc(-c4ccc(-c5cccc6ccccc56)cc4)nc(-c4cccc5oc6ccccc6c45)n3)c2)cc1 ; c1ccc(-c2cc(-c3nc(-c4ccc(-c5cccc6ccccc56)cc4)nc(-c4cccc5oc6ccccc6c45)n3)cc(-c3cccc4oc(-c5ccccc5)nc34)c2)cc1] 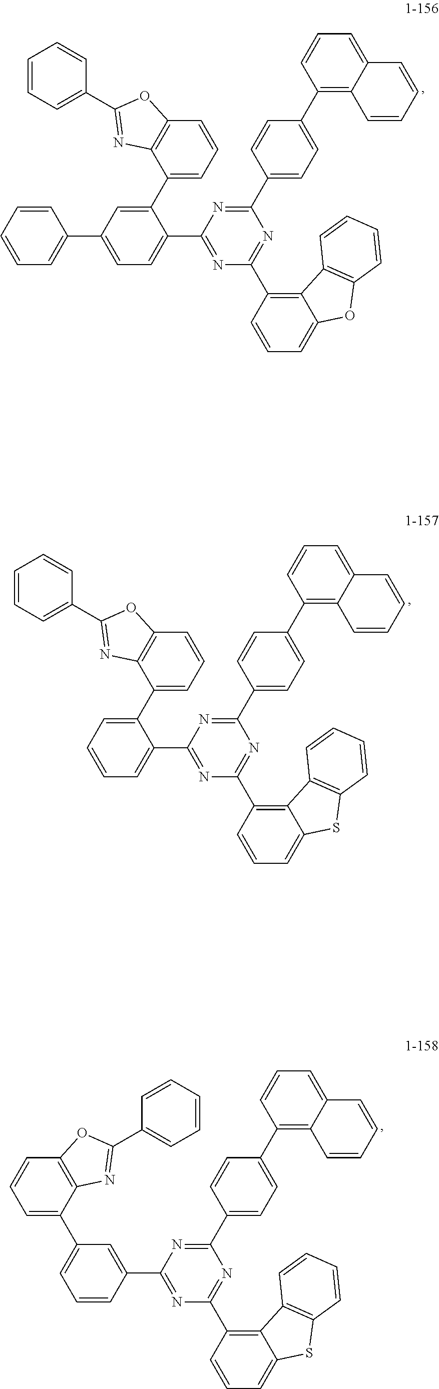 [SMILES: c1ccc(-c2ccc(-c3nc(-c4ccc(-c5cccc6ccccc56)cc4)nc(-c4cccc5oc6ccccc6c45)n3)c(-c3cccc4oc(-c5ccccc5)nc34)c2)cc1 ; c1ccc(-c2nc3c(-c4ccccc4-c4nc(-c5ccc(-c6cccc7ccccc67)cc5)nc(-c5cccc6sc7ccccc7c56)n4)cccc3o2)cc1 ; c1ccc(-c2nc3c(-c4cccc(-c5nc(-c6ccc(-c7cccc8ccccc78)cc6)nc(-c6cccc7sc8ccccc8c67)n5)c4)cccc3o2)cc1] 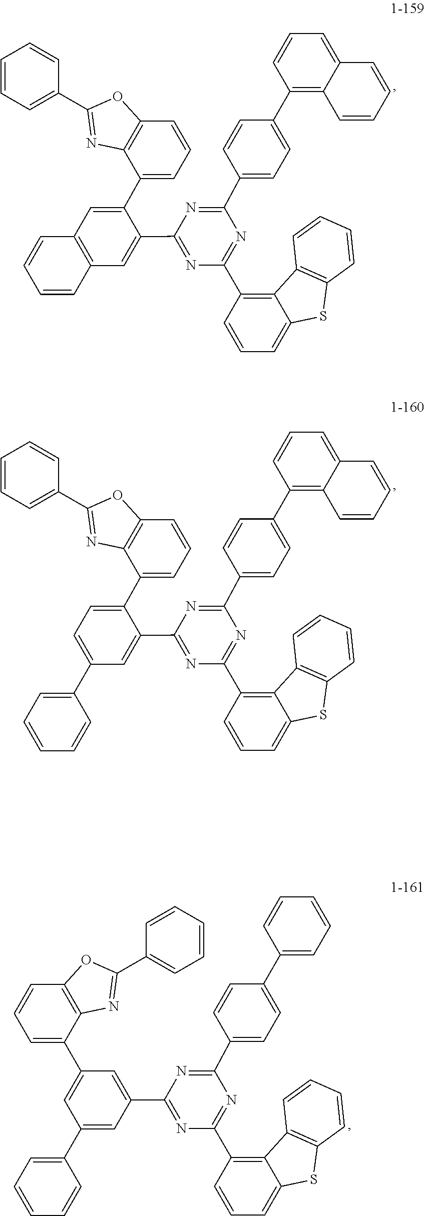 [SMILES: c1ccc(-c2nc3c(-c4cc5ccccc5cc4-c4nc(-c5ccc(-c6cccc7ccccc67)cc5)nc(-c5cccc6sc7ccccc7c56)n4)cccc3o2)cc1 ; c1ccc(-c2ccc(-c3cccc4oc(-c5ccccc5)nc34)c(-c3nc(-c4ccc(-c5cccc6ccccc56)cc4)nc(-c4cccc5sc6ccccc6c45)n3)c2)cc1 ; c1ccc(-c2ccc(-c3nc(-c4cc(-c5ccccc5)cc(-c5cccc6oc(-c7ccccc7)nc56)c4)nc(-c4cccc5sc6ccccc6c45)n3)cc2)cc1] 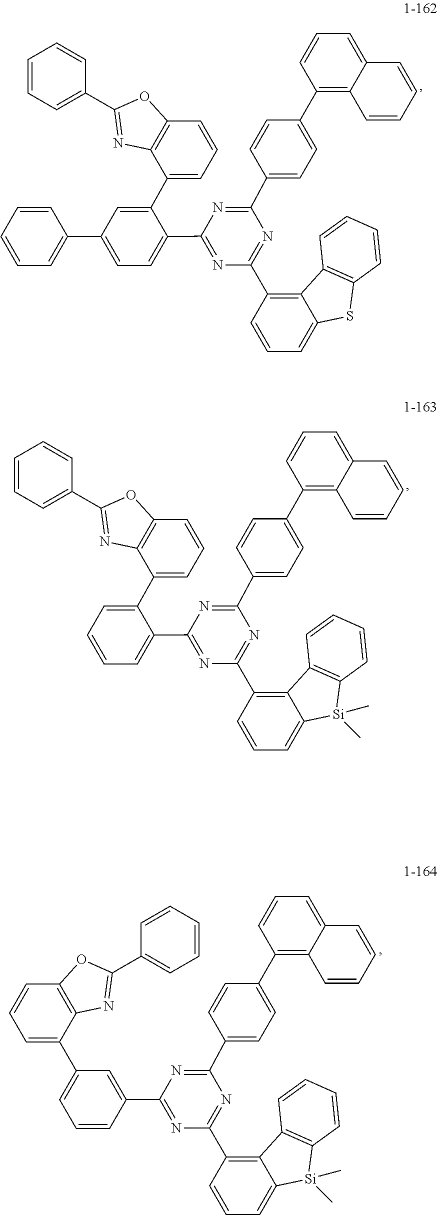 [SMILES: c1ccc(-c2ccc(-c3nc(-c4ccc(-c5cccc6ccccc56)cc4)nc(-c4cccc5sc6ccccc6c45)n3)c(-c3cccc4oc(-c5ccccc5)nc34)c2)cc1 ; C[Si]1(C)c2ccccc2-c2c(-c3nc(-c4ccc(-c5cccc6ccccc56)cc4)nc(-c4ccccc4-c4cccc5oc(-c6ccccc6)nc45)n3)cccc21 ; C[Si]1(C)c2ccccc2-c2c(-c3nc(-c4ccc(-c5cccc6ccccc56)cc4)nc(-c4cccc(-c5cccc6oc(-c7ccccc7)nc56)c4)n3)cccc21] 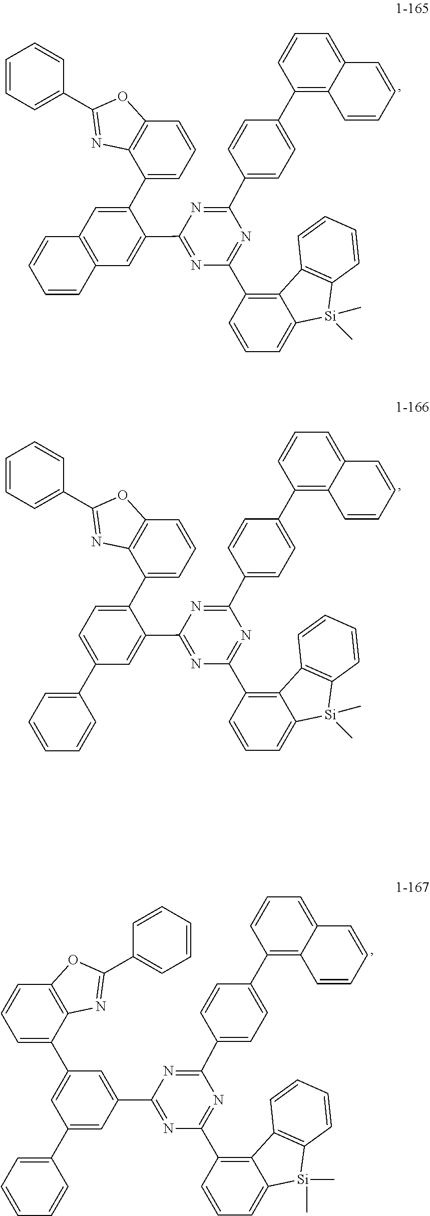 [SMILES: C[Si]1(C)c2ccccc2-c2c(-c3nc(-c4ccc(-c5cccc6ccccc56)cc4)nc(-c4cc5ccccc5cc4-c4cccc5oc(-c6ccccc6)nc45)n3)cccc21 ; C[Si]1(C)c2ccccc2-c2c(-c3nc(-c4ccc(-c5cccc6ccccc56)cc4)nc(-c4cc(-c5ccccc5)ccc4-c4cccc5oc(-c6ccccc6)nc45)n3)cccc21 ; C[Si]1(C)c2ccccc2-c2c(-c3nc(-c4ccc(-c5cccc6ccccc56)cc4)nc(-c4cc(-c5ccccc5)cc(-c5cccc6oc(-c7ccccc7)nc56)c4)n3)cccc21] 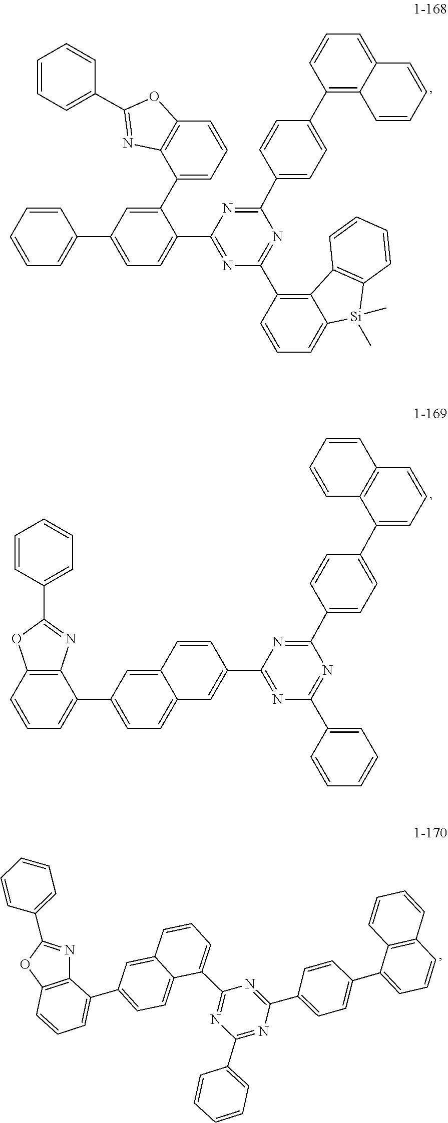 [SMILES: C[Si]1(C)c2ccccc2-c2c(-c3nc(-c4ccc(-c5cccc6ccccc56)cc4)nc(-c4ccc(-c5ccccc5)cc4-c4cccc5oc(-c6ccccc6)nc45)n3)cccc21 ; c1ccc(-c2nc(-c3ccc(-c4cccc5ccccc45)cc3)nc(-c3ccc4cc(-c5cccc6oc(-c7ccccc7)nc56)ccc4c3)n2)cc1 ; c1ccc(-c2nc(-c3ccc(-c4cccc5ccccc45)cc3)nc(-c3cccc4cc(-c5cccc6oc(-c7ccccc7)nc56)ccc34)n2)cc1] 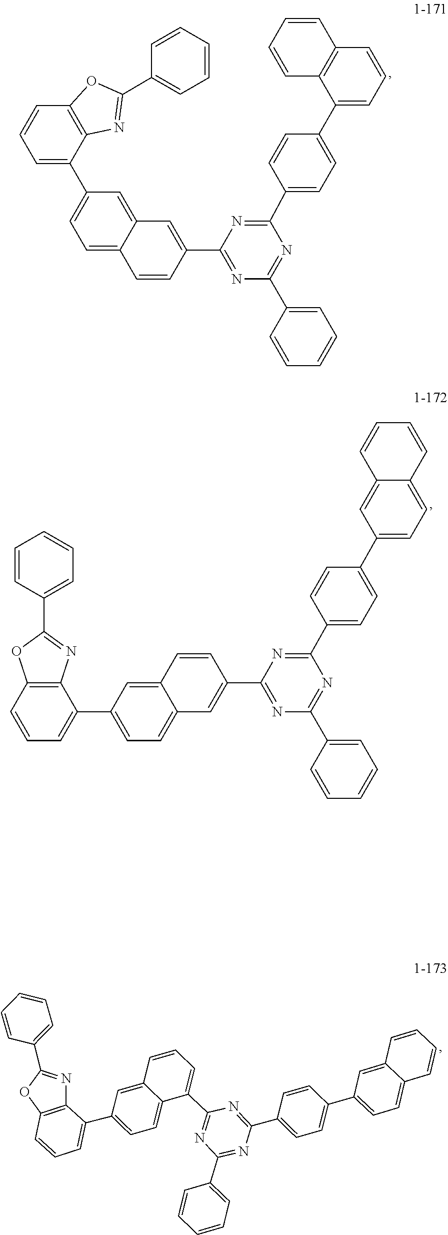 [SMILES: c1ccc(-c2nc(-c3ccc(-c4cccc5ccccc45)cc3)nc(-c3ccc4ccc(-c5cccc6oc(-c7ccccc7)nc56)cc4c3)n2)cc1 ; c1ccc(-c2nc(-c3ccc(-c4ccc5ccccc5c4)cc3)nc(-c3ccc4cc(-c5cccc6oc(-c7ccccc7)nc56)ccc4c3)n2)cc1 ; c1ccc(-c2nc(-c3ccc(-c4ccc5ccccc5c4)cc3)nc(-c3cccc4cc(-c5cccc6oc(-c7ccccc7)nc56)ccc34)n2)cc1] 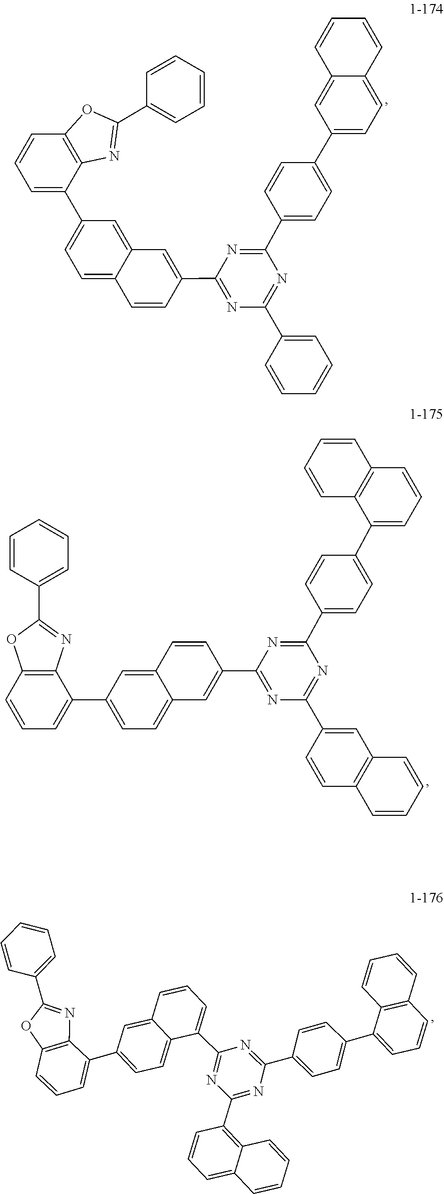 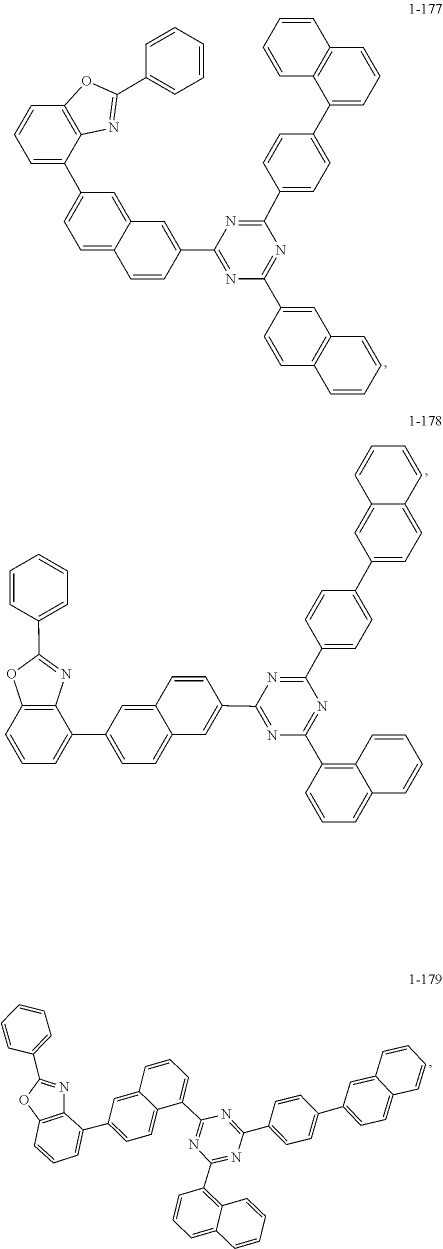 [SMILES: c1ccc(-c2nc3c(-c4ccc5ccc(-c6nc(-c7ccc(-c8cccc9ccccc89)cc7)nc(-c7ccc8ccccc8c7)n6)cc5c4)cccc3o2)cc1 ; c1ccc(-c2nc3c(-c4ccc5cc(-c6nc(-c7ccc(-c8ccc9ccccc9c8)cc7)nc(-c7cccc8ccccc78)n6)ccc5c4)cccc3o2)cc1 ; c1ccc(-c2nc3c(-c4ccc5c(-c6nc(-c7ccc(-c8ccc9ccccc9c8)cc7)nc(-c7cccc8ccccc78)n6)cccc5c4)cccc3o2)cc1] 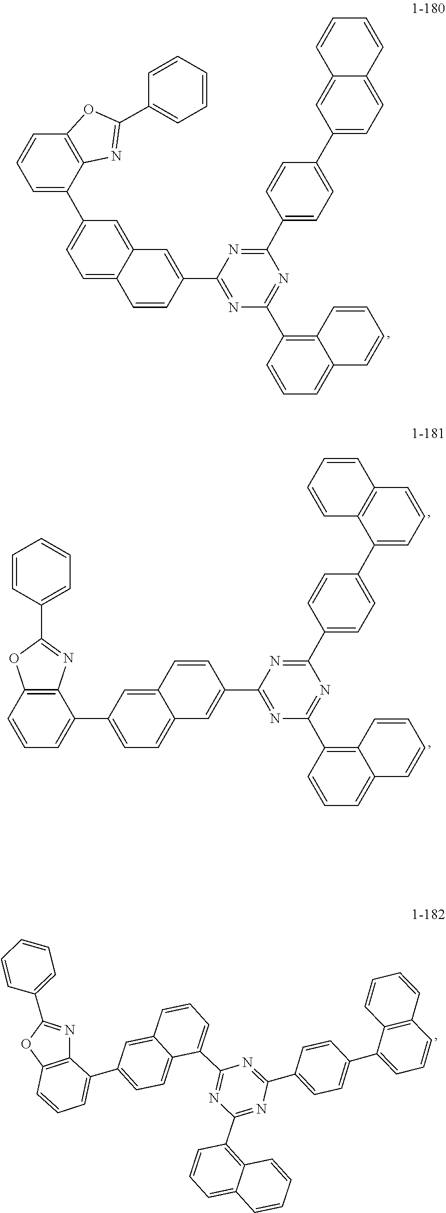 [SMILES: c1ccc(-c2nc3c(-c4ccc5ccc(-c6nc(-c7ccc(-c8ccc9ccccc9c8)cc7)nc(-c7cccc8ccccc78)n6)cc5c4)cccc3o2)cc1 ; c1ccc(-c2nc3c(-c4ccc5cc(-c6nc(-c7ccc(-c8cccc9ccccc89)cc7)nc(-c7cccc8ccccc78)n6)ccc5c4)cccc3o2)cc1 ; c1ccc(-c2nc3c(-c4ccc5c(-c6nc(-c7ccc(-c8cccc9ccccc89)cc7)nc(-c7cccc8ccccc78)n6)cccc5c4)cccc3o2)cc1] 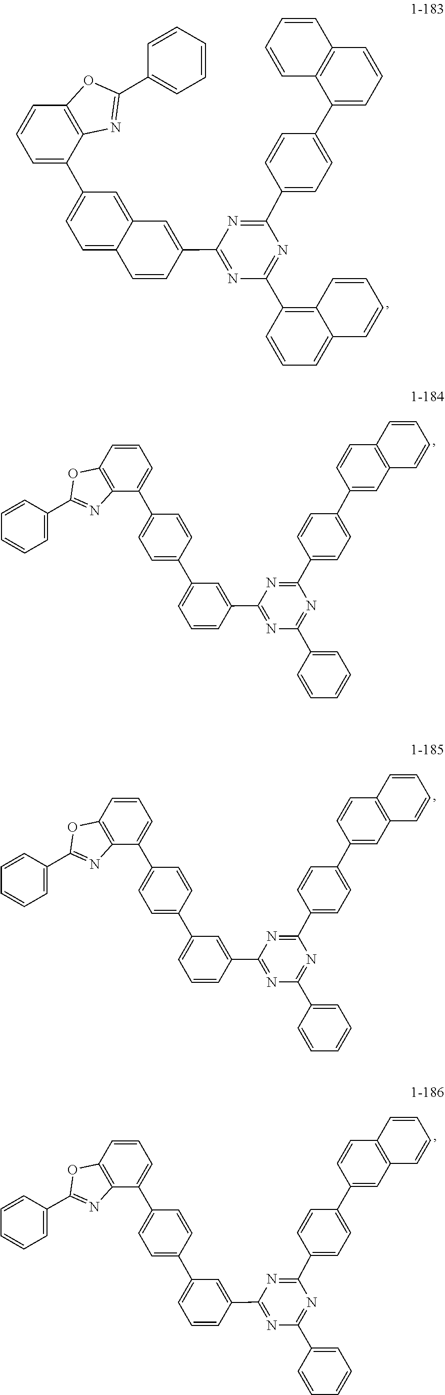 [SMILES: c1ccc(-c2nc3c(-c4ccc5ccc(-c6nc(-c7ccc(-c8cccc9ccccc89)cc7)nc(-c7cccc8ccccc78)n6)cc5c4)cccc3o2)cc1 ; c1ccc(-c2nc(-c3ccc(-c4ccc5ccccc5c4)cc3)nc(-c3cccc(-c4ccc(-c5cccc6oc(-c7ccccc7)nc56)cc4)c3)n2)cc1 ; c1ccc(-c2nc(-c3ccc(-c4ccc5ccccc5c4)cc3)nc(-c3cccc(-c4ccc(-c5cccc6oc(-c7ccccc7)nc56)cc4)c3)n2)cc1 ; c1ccc(-c2nc(-c3ccc(-c4ccc5ccccc5c4)cc3)nc(-c3cccc(-c4ccc(-c5cccc6oc(-c7ccccc7)nc56)cc4)c3)n2)cc1] 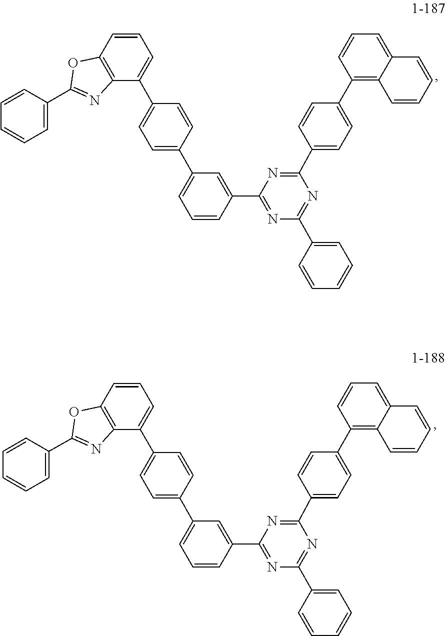 [SMILES: c1ccc(-c2nc(-c3ccc(-c4cccc5ccccc45)cc3)nc(-c3cccc(-c4ccc(-c5cccc6oc(-c7ccccc7)nc56)cc4)c3)n2)cc1 ; c1ccc(-c2nc(-c3ccc(-c4cccc5ccccc45)cc3)nc(-c3cccc(-c4ccc(-c5cccc6oc(-c7ccccc7)nc56)cc4)c3)n2)cc1] 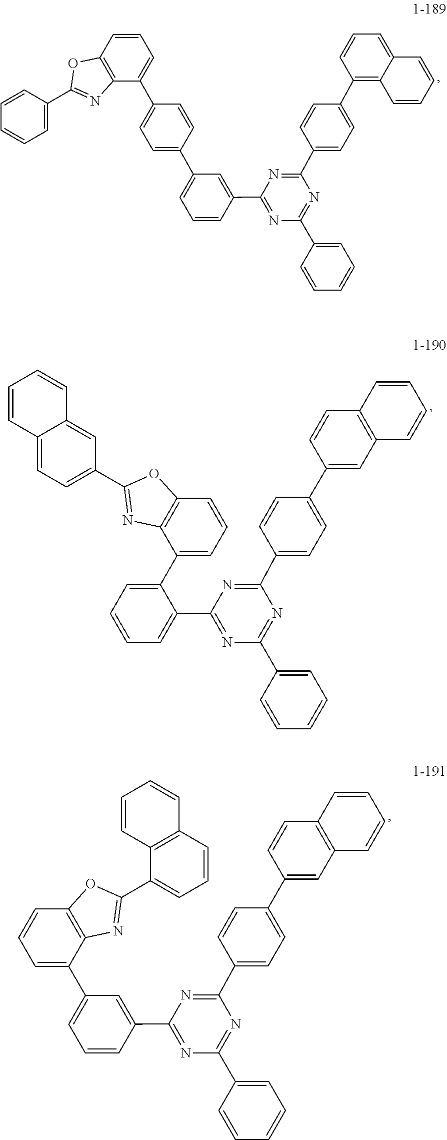 [SMILES: c1ccc(-c2nc(-c3ccc(-c4cccc5ccccc45)cc3)nc(-c3cccc(-c4ccc(-c5cccc6oc(-c7ccccc7)nc56)cc4)c3)n2)cc1 ; c1ccc(-c2nc(-c3ccc(-c4ccc5ccccc5c4)cc3)nc(-c3ccccc3-c3cccc4oc(-c5ccc6ccccc6c5)nc34)n2)cc1 ; c1ccc(-c2nc(-c3ccc(-c4ccc5ccccc5c4)cc3)nc(-c3cccc(-c4cccc5oc(-c6cccc7ccccc67)nc45)c3)n2)cc1] 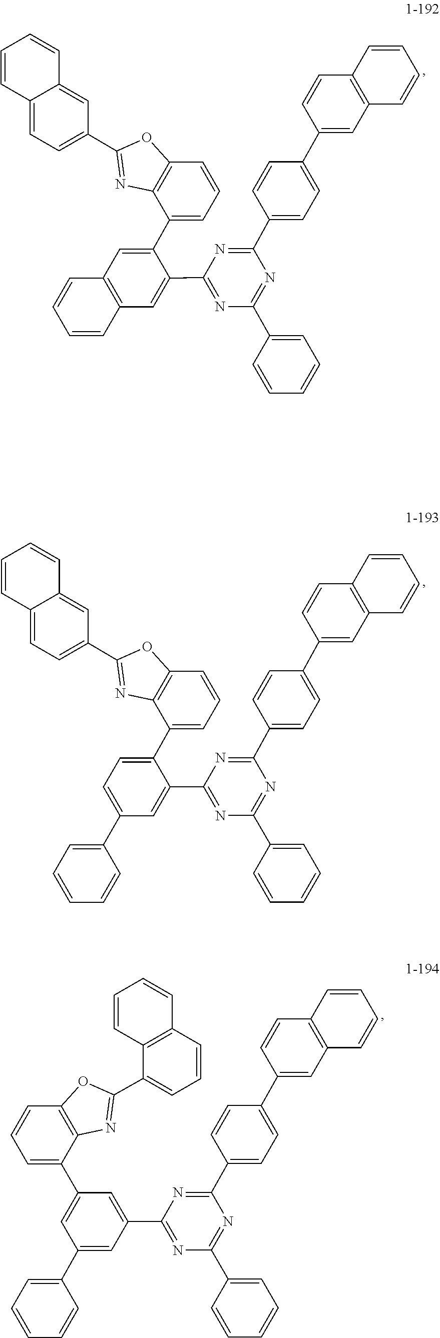 [SMILES: c1ccc(-c2nc(-c3ccc(-c4ccc5ccccc5c4)cc3)nc(-c3cc4ccccc4cc3-c3cccc4oc(-c5ccc6ccccc6c5)nc34)n2)cc1 ; c1ccc(-c2ccc(-c3cccc4oc(-c5ccc6ccccc6c5)nc34)c(-c3nc(-c4ccccc4)nc(-c4ccc(-c5ccc6ccccc6c5)cc4)n3)c2)cc1 ; c1ccc(-c2cc(-c3nc(-c4ccccc4)nc(-c4ccc(-c5ccc6ccccc6c5)cc4)n3)cc(-c3cccc4oc(-c5cccc6ccccc56)nc34)c2)cc1] 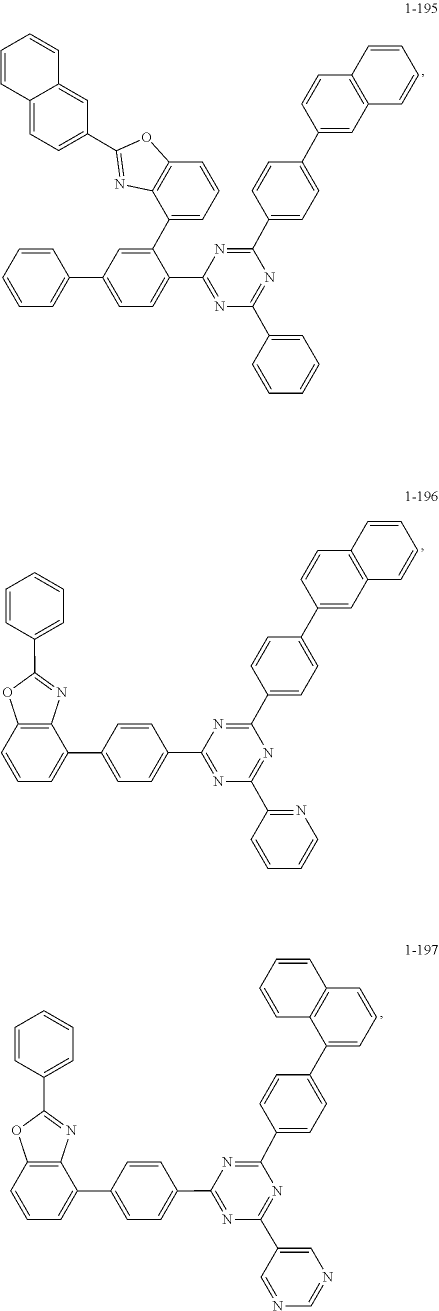 [SMILES: c1ccc(-c2ccc(-c3nc(-c4ccccc4)nc(-c4ccc(-c5ccc6ccccc6c5)cc4)n3)c(-c3cccc4oc(-c5ccc6ccccc6c5)nc34)c2)cc1 ; c1ccc(-c2nc3c(-c4ccc(-c5nc(-c6ccc(-c7ccc8ccccc8c7)cc6)nc(-c6ccccn6)n5)cc4)cccc3o2)cc1 ; c1ccc(-c2nc3c(-c4ccc(-c5nc(-c6ccc(-c7cccc8ccccc78)cc6)nc(-c6cncnc6)n5)cc4)cccc3o2)cc1] 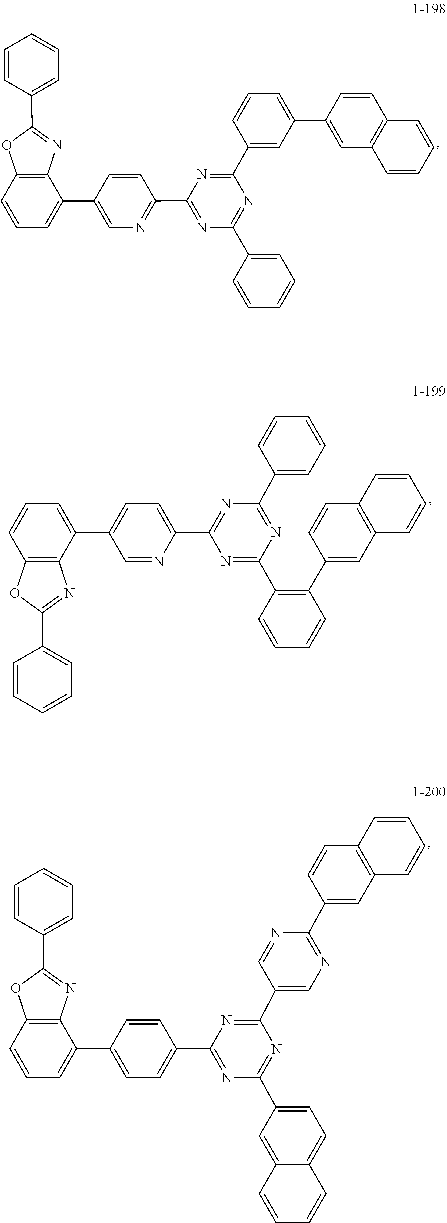 [SMILES: c1ccc(-c2nc(-c3cccc(-c4ccc5ccccc5c4)c3)nc(-c3ccc(-c4cccc5oc(-c6ccccc6)nc45)cn3)n2)cc1 ; c1ccc(-c2nc(-c3ccc(-c4cccc5oc(-c6ccccc6)nc45)cn3)nc(-c3ccccc3-c3ccc4ccccc4c3)n2)cc1 ; c1ccc(-c2nc3c(-c4ccc(-c5nc(-c6cnc(-c7ccc8ccccc8c7)nc6)nc(-c6ccc7ccccc7c6)n5)cc4)cccc3o2)cc1] 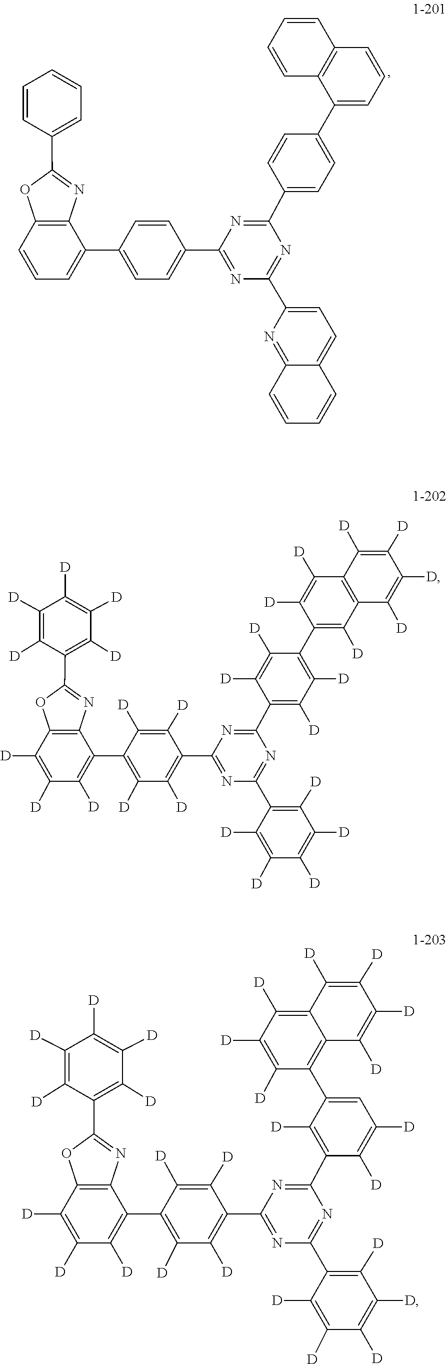 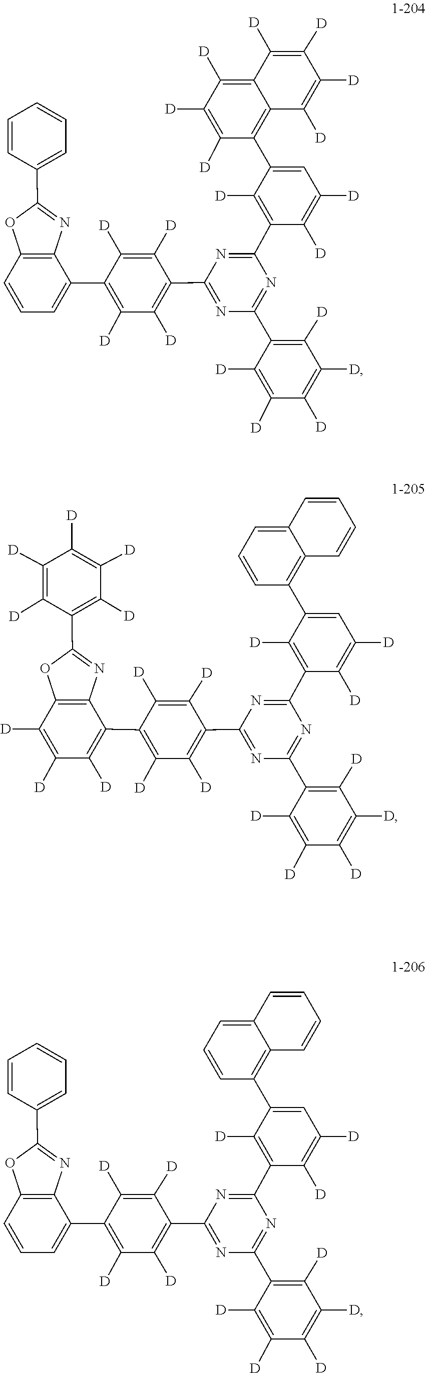 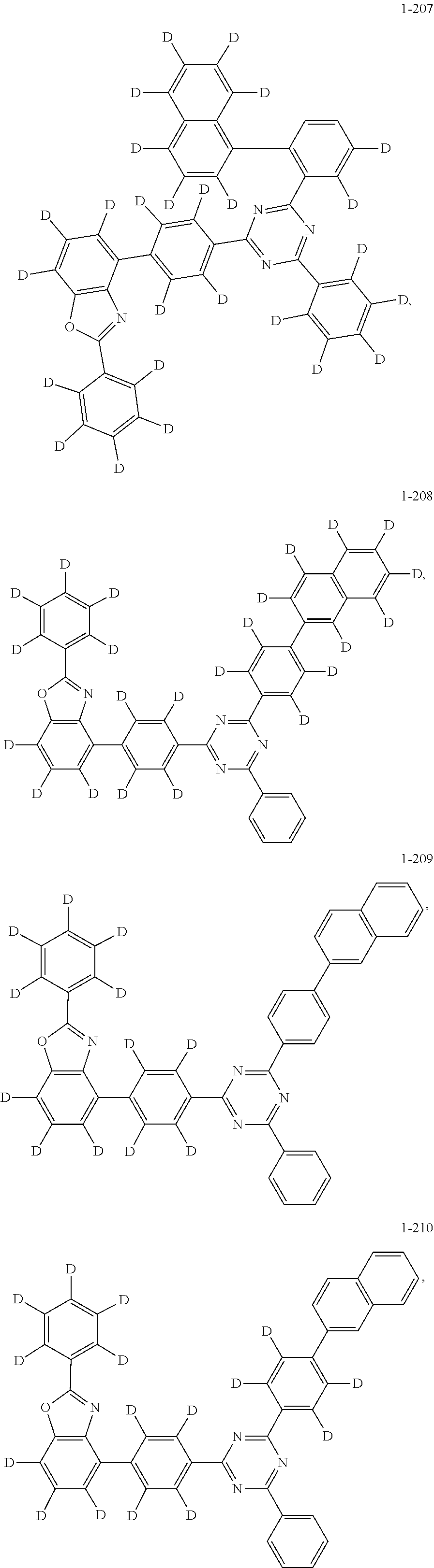 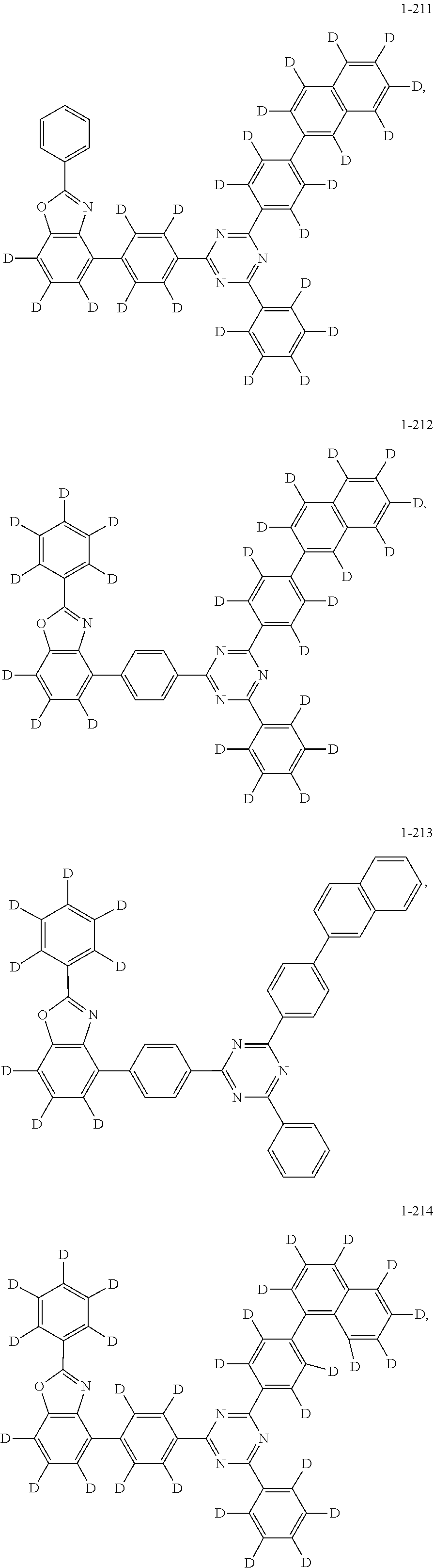 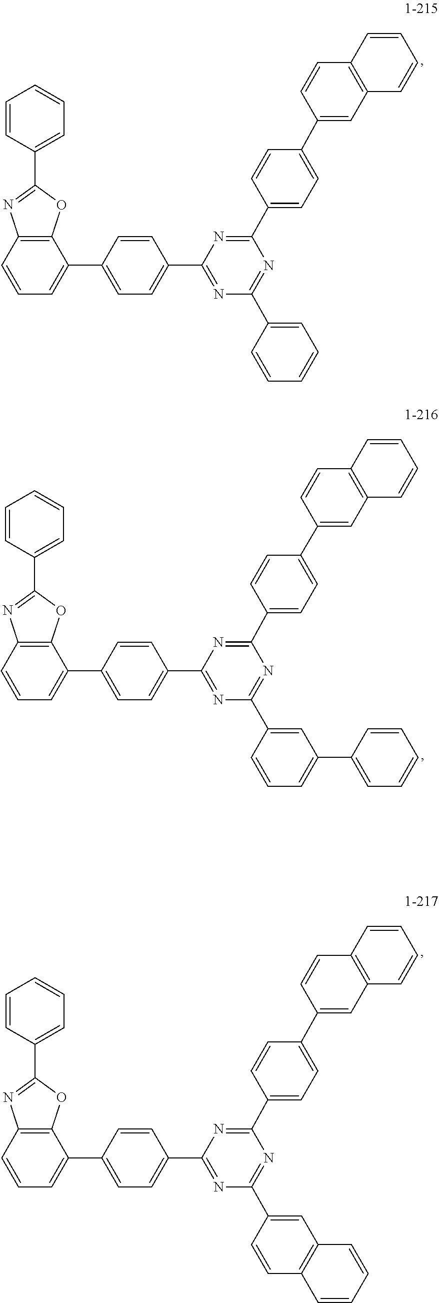 [SMILES: c1ccc(-c2nc(-c3ccc(-c4ccc5ccccc5c4)cc3)nc(-c3ccc(-c4cccc5nc(-c6ccccc6)oc45)cc3)n2)cc1 ; c1ccc(-c2cccc(-c3nc(-c4ccc(-c5ccc6ccccc6c5)cc4)nc(-c4ccc(-c5cccc6nc(-c7ccccc7)oc56)cc4)n3)c2)cc1 ; c1ccc(-c2nc3cccc(-c4ccc(-c5nc(-c6ccc(-c7ccc8ccccc8c7)cc6)nc(-c6ccc7ccccc7c6)n5)cc4)c3o2)cc1] 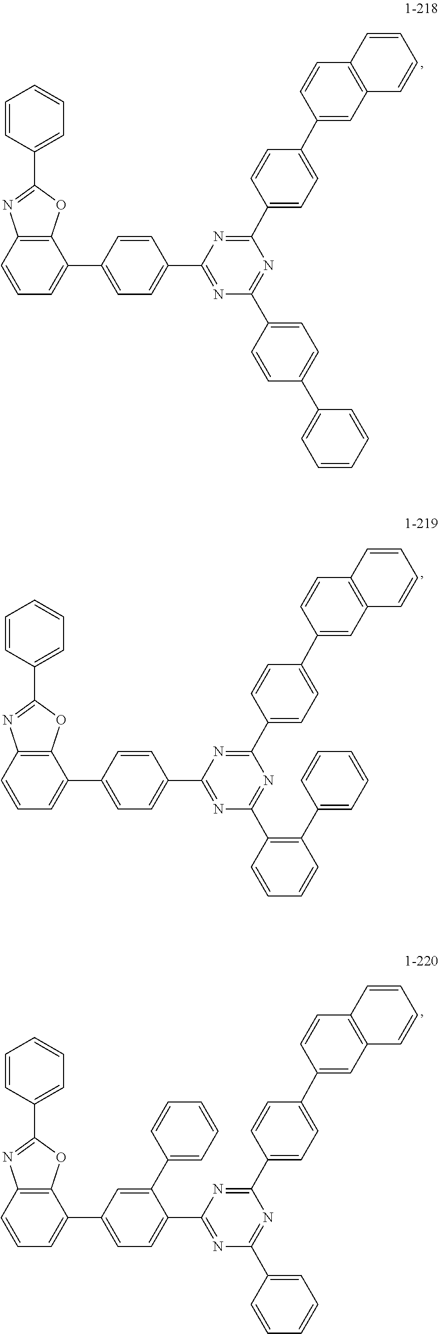 [SMILES: c1ccc(-c2ccc(-c3nc(-c4ccc(-c5ccc6ccccc6c5)cc4)nc(-c4ccc(-c5cccc6nc(-c7ccccc7)oc56)cc4)n3)cc2)cc1 ; c1ccc(-c2nc3cccc(-c4ccc(-c5nc(-c6ccc(-c7ccc8ccccc8c7)cc6)nc(-c6ccccc6-c6ccccc6)n5)cc4)c3o2)cc1 ; c1ccc(-c2nc(-c3ccc(-c4ccc5ccccc5c4)cc3)nc(-c3ccc(-c4cccc5nc(-c6ccccc6)oc45)cc3-c3ccccc3)n2)cc1] 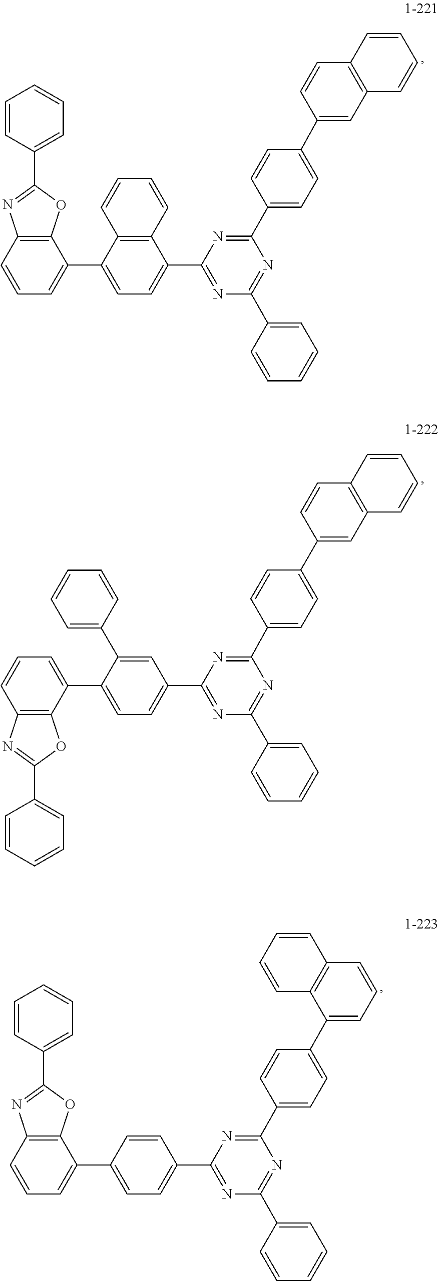 [SMILES: c1ccc(-c2nc(-c3ccc(-c4ccc5ccccc5c4)cc3)nc(-c3ccc(-c4cccc5nc(-c6ccccc6)oc45)c4ccccc34)n2)cc1 ; c1ccc(-c2nc(-c3ccc(-c4ccc5ccccc5c4)cc3)nc(-c3ccc(-c4cccc5nc(-c6ccccc6)oc45)c(-c4ccccc4)c3)n2)cc1 ; c1ccc(-c2nc(-c3ccc(-c4cccc5ccccc45)cc3)nc(-c3ccc(-c4cccc5nc(-c6ccccc6)oc45)cc3)n2)cc1] 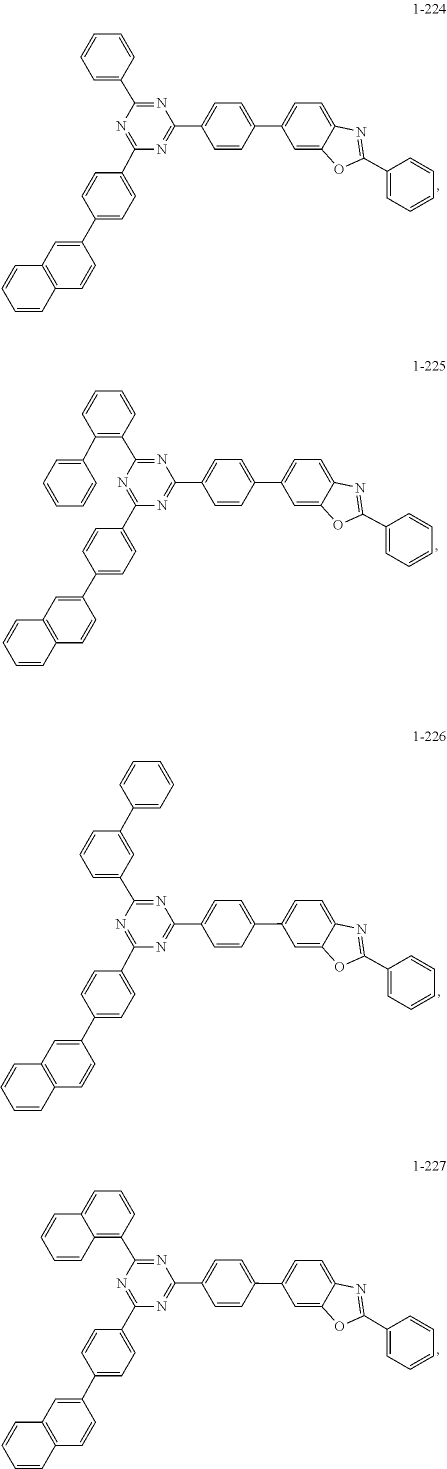 [SMILES: c1ccc(-c2nc(-c3ccc(-c4ccc5ccccc5c4)cc3)nc(-c3ccc(-c4ccc5nc(-c6ccccc6)oc5c4)cc3)n2)cc1 ; c1ccc(-c2nc3ccc(-c4ccc(-c5nc(-c6ccc(-c7ccc8ccccc8c7)cc6)nc(-c6ccccc6-c6ccccc6)n5)cc4)cc3o2)cc1 ; c1ccc(-c2cccc(-c3nc(-c4ccc(-c5ccc6ccccc6c5)cc4)nc(-c4ccc(-c5ccc6nc(-c7ccccc7)oc6c5)cc4)n3)c2)cc1 ; c1ccc(-c2nc3ccc(-c4ccc(-c5nc(-c6ccc(-c7ccc8ccccc8c7)cc6)nc(-c6cccc7ccccc67)n5)cc4)cc3o2)cc1] 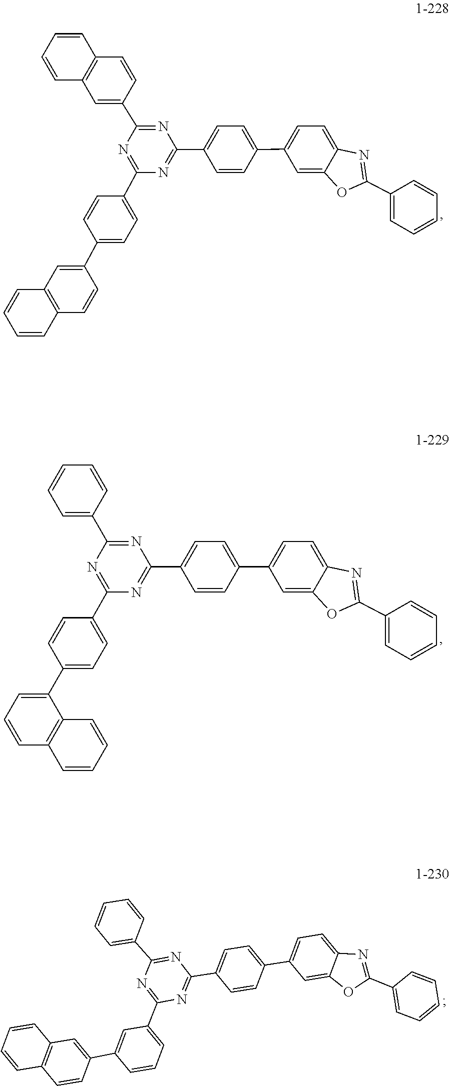 [SMILES: c1ccc(-c2nc3ccc(-c4ccc(-c5nc(-c6ccc(-c7ccc8ccccc8c7)cc6)nc(-c6ccc7ccccc7c6)n5)cc4)cc3o2)cc1 ; c1ccc(-c2nc(-c3ccc(-c4ccc5nc(-c6ccccc6)oc5c4)cc3)nc(-c3ccc(-c4cccc5ccccc45)cc3)n2)cc1 ; c1ccc(-c2nc(-c3ccc(-c4ccc5nc(-c6ccccc6)oc5c4)cc3)nc(-c3cccc(-c4ccc5ccccc5c4)c3)n2)cc1]
wherein optionally, hydrogens in the structures of Compound 1-1 to Compound 1-230 can be partially or fully substituted with deuterium.

10. An organic electroluminescent device, comprising:
an anode,
a cathode, and
an organic layer disposed between the anode and the cathode, wherein the organic layer comprises the compound of claim 1.

11. The organic electroluminescent device of claim 10, wherein the organic layer is an emissive layer, and the compound is a host material.

12. The organic electroluminescent device of claim 11, wherein the organic layer is the emissive layer and further comprises a second compound, and the second compound is a host material and has a structure represented by any one of Formula 2-1 to Formula 2-3: 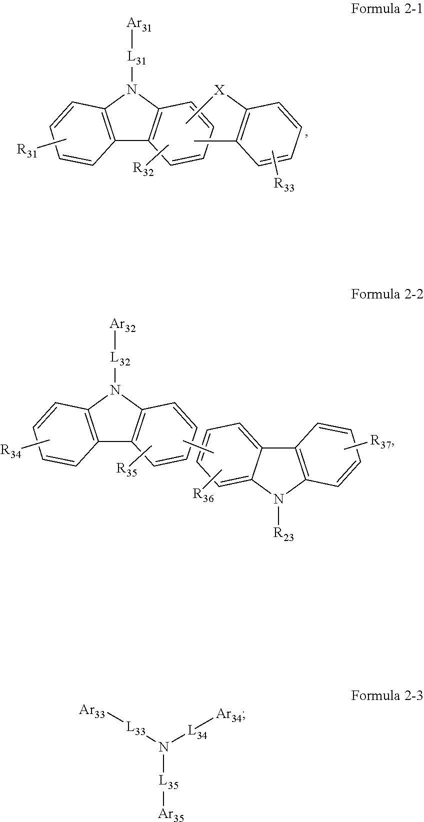
wherein
in Formula 2-1 to Formula 2-3, Ar₃₁ to Ar₃₅ are, at each occurrence identically or differently, selected from substituted or unsubstituted aryl having 6 to 30 carbon atoms or substituted or unsubstituted heteroaryl having 3 to 30 carbon atoms;
L₃₁ to L₃₅ are, at each occurrence identically or differently, selected from a single bond, substituted or unsubstituted arylene having 6 to 30 carbon atoms, substituted or unsubstituted heteroarylene having 3 to 30 carbon atoms, or a combination thereof;
X is, at each occurrence identically or differently, selected from O, S, CR₂₁R₂₂, or NR₂₃;
R₃₁ to R₃₇ represent, at each occurrence identically or differently, mono-substitution, multiple substitutions, or non-substitution;
R₃₁ to R₃₇ and R₂₁ to R₂₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₃₁ to R₃₇ can be optionally joined to form a ring;
preferably, the second compound is selected from the group consisting of the following compounds:

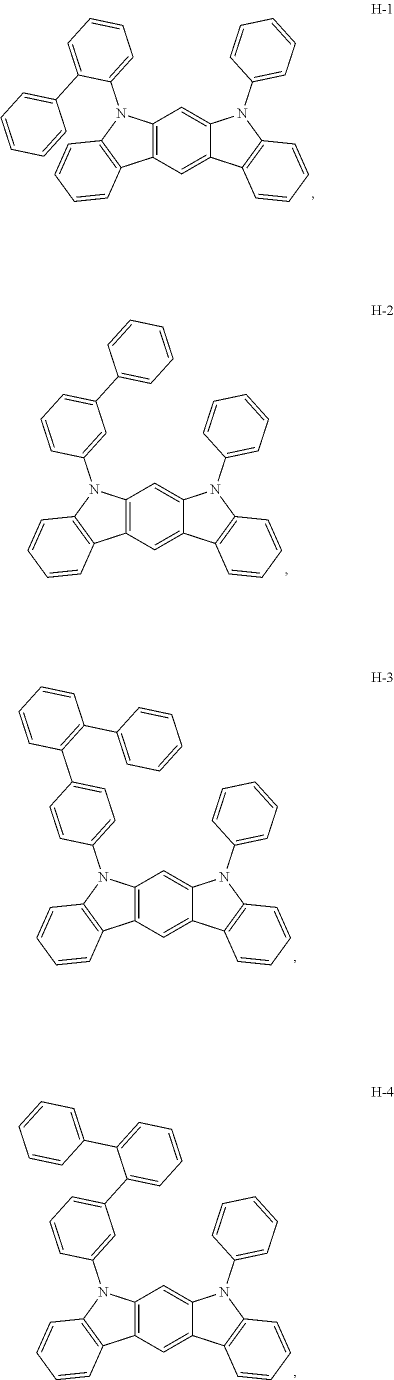 [SMILES: c1ccc(-c2ccccc2-n2c3ccccc3c3cc4c5ccccc5n(-c5ccccc5)c4cc32)cc1 ; c1ccc(-c2cccc(-n3c4ccccc4c4cc5c6ccccc6n(-c6ccccc6)c5cc43)c2)cc1 ; c1ccc(-c2ccccc2-c2ccc(-n3c4ccccc4c4cc5c6ccccc6n(-c6ccccc6)c5cc43)cc2)cc1 ; c1ccc(-c2ccccc2-c2cccc(-n3c4ccccc4c4cc5c6ccccc6n(-c6ccccc6)c5cc43)c2)cc1]

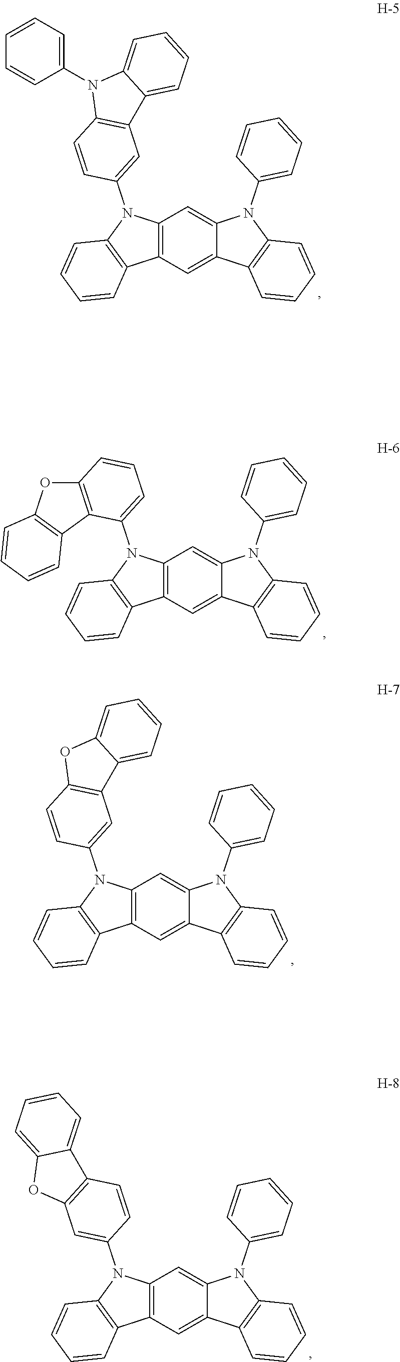 [SMILES: c1ccc(-n2c3ccccc3c3cc(-n4c5ccccc5c5cc6c7ccccc7n(-c7ccccc7)c6cc54)ccc32)cc1 ; c1ccc(-n2c3ccccc3c3cc4c5ccccc5n(-c5cccc6oc7ccccc7c56)c4cc32)cc1 ; c1ccc(-n2c3ccccc3c3cc4c5ccccc5n(-c5ccc6oc7ccccc7c6c5)c4cc32)cc1 ; c1ccc(-n2c3ccccc3c3cc4c5ccccc5n(-c5ccc6c(c5)oc5ccccc56)c4cc32)cc1]

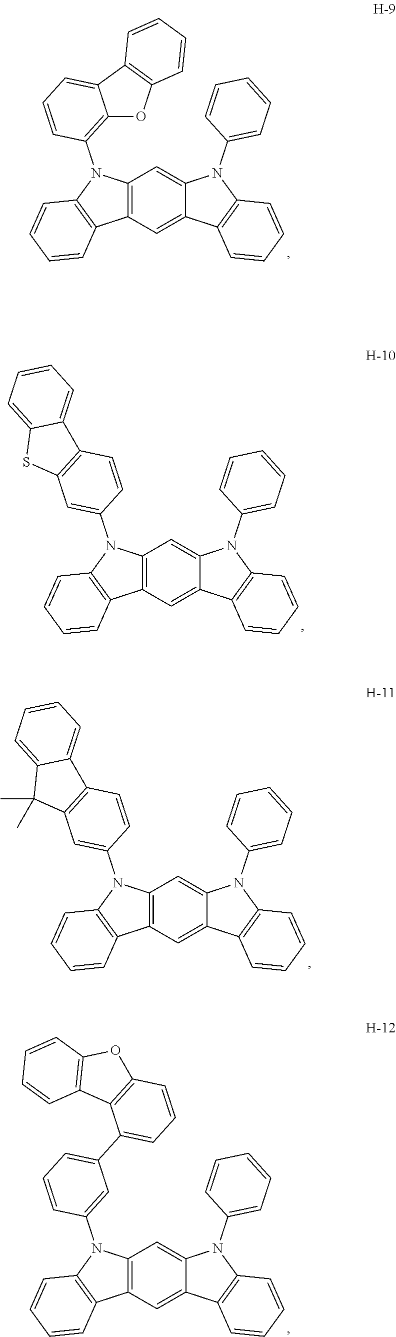 [SMILES: c1ccc(-n2c3ccccc3c3cc4c5ccccc5n(-c5cccc6c5oc5ccccc56)c4cc32)cc1 ; c1ccc(-n2c3ccccc3c3cc4c5ccccc5n(-c5ccc6c(c5)sc5ccccc56)c4cc32)cc1 ; CC1(C)c2ccccc2-c2ccc(-n3c4ccccc4c4cc5c6ccccc6n(-c6ccccc6)c5cc43)cc21 ; c1ccc(-n2c3ccccc3c3cc4c5ccccc5n(-c5cccc(-c6cccc7oc8ccccc8c67)c5)c4cc32)cc1]

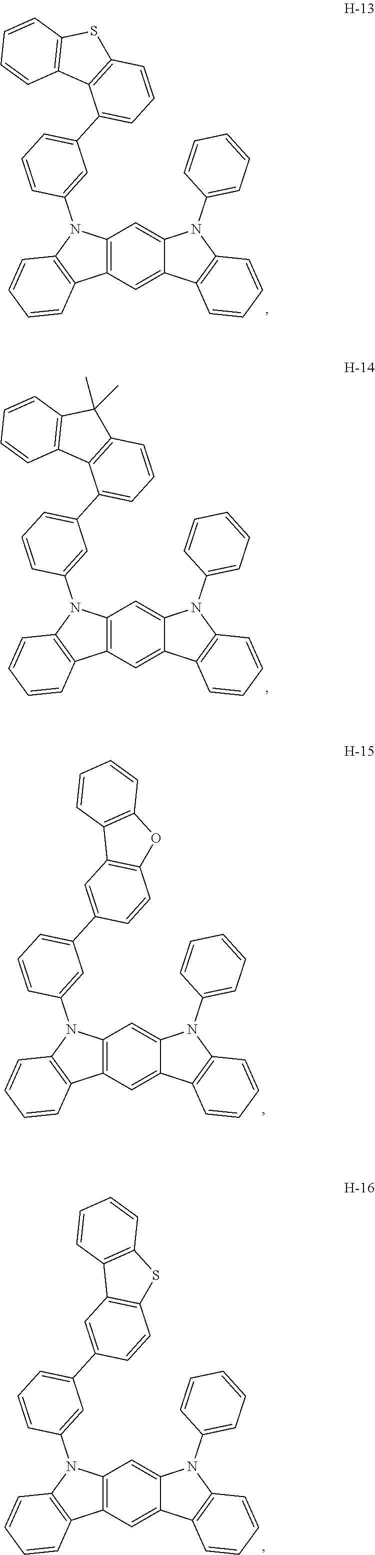 [SMILES: c1ccc(-n2c3ccccc3c3cc4c5ccccc5n(-c5cccc(-c6cccc7sc8ccccc8c67)c5)c4cc32)cc1 ; CC1(C)c2ccccc2-c2c(-c3cccc(-n4c5ccccc5c5cc6c7ccccc7n(-c7ccccc7)c6cc54)c3)cccc21 ; c1ccc(-n2c3ccccc3c3cc4c5ccccc5n(-c5cccc(-c6ccc7oc8ccccc8c7c6)c5)c4cc32)cc1 ; c1ccc(-n2c3ccccc3c3cc4c5ccccc5n(-c5cccc(-c6ccc7sc8ccccc8c7c6)c5)c4cc32)cc1]

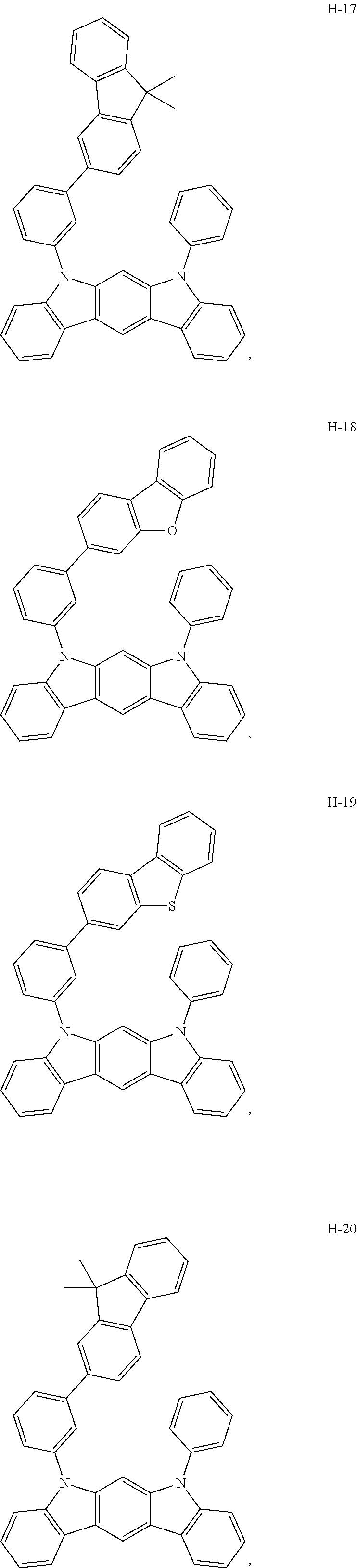 [SMILES: CC1(C)c2ccccc2-c2cc(-c3cccc(-n4c5ccccc5c5cc6c7ccccc7n(-c7ccccc7)c6cc54)c3)ccc21 ; c1ccc(-n2c3ccccc3c3cc4c5ccccc5n(-c5cccc(-c6ccc7c(c6)oc6ccccc67)c5)c4cc32)cc1 ; c1ccc(-n2c3ccccc3c3cc4c5ccccc5n(-c5cccc(-c6ccc7c(c6)sc6ccccc67)c5)c4cc32)cc1 ; CC1(C)c2ccccc2-c2ccc(-c3cccc(-n4c5ccccc5c5cc6c7ccccc7n(-c7ccccc7)c6cc54)c3)cc21]

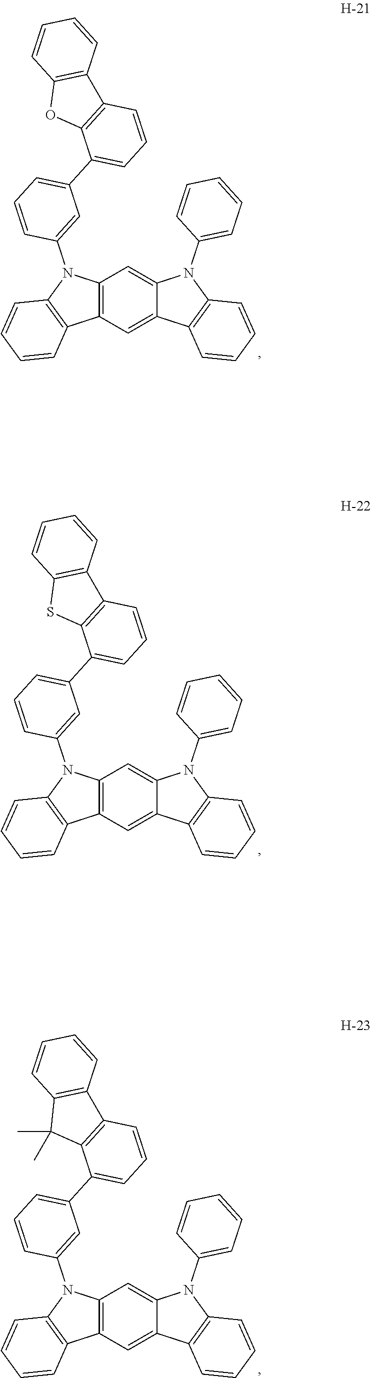 [SMILES: c1ccc(-n2c3ccccc3c3cc4c5ccccc5n(-c5cccc(-c6cccc7c6oc6ccccc67)c5)c4cc32)cc1 ; c1ccc(-n2c3ccccc3c3cc4c5ccccc5n(-c5cccc(-c6cccc7c6sc6ccccc67)c5)c4cc32)cc1 ; CC1(C)c2ccccc2-c2cccc(-c3cccc(-n4c5ccccc5c5cc6c7ccccc7n(-c7ccccc7)c6cc54)c3)c21]

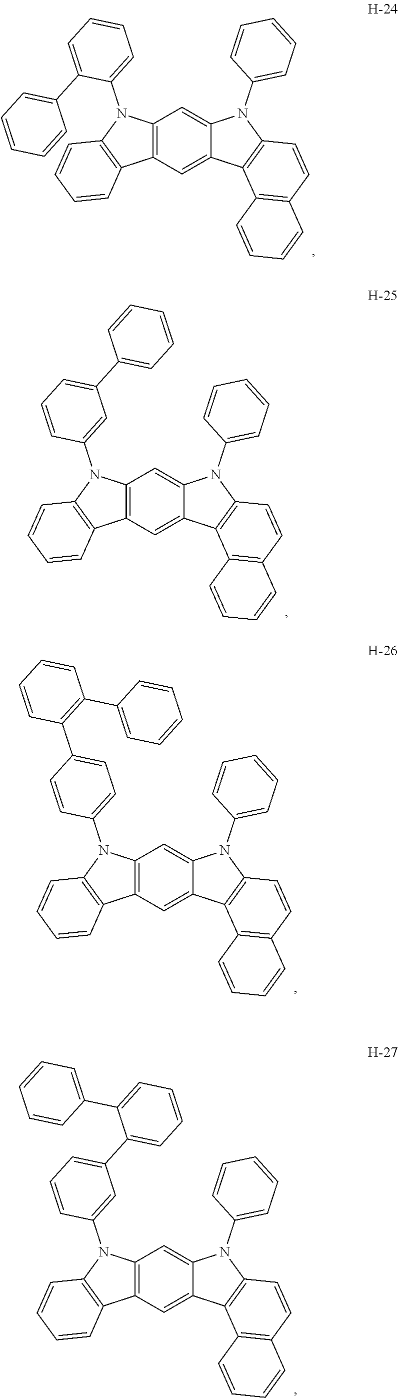 [SMILES: c1ccc(-c2ccccc2-n2c3ccccc3c3cc4c5c6ccccc6ccc5n(-c5ccccc5)c4cc32)cc1 ; c1ccc(-c2cccc(-n3c4ccccc4c4cc5c6c7ccccc7ccc6n(-c6ccccc6)c5cc43)c2)cc1 ; c1ccc(-c2ccccc2-c2ccc(-n3c4ccccc4c4cc5c6c7ccccc7ccc6n(-c6ccccc6)c5cc43)cc2)cc1 ; c1ccc(-c2ccccc2-c2cccc(-n3c4ccccc4c4cc5c6c7ccccc7ccc6n(-c6ccccc6)c5cc43)c2)cc1]

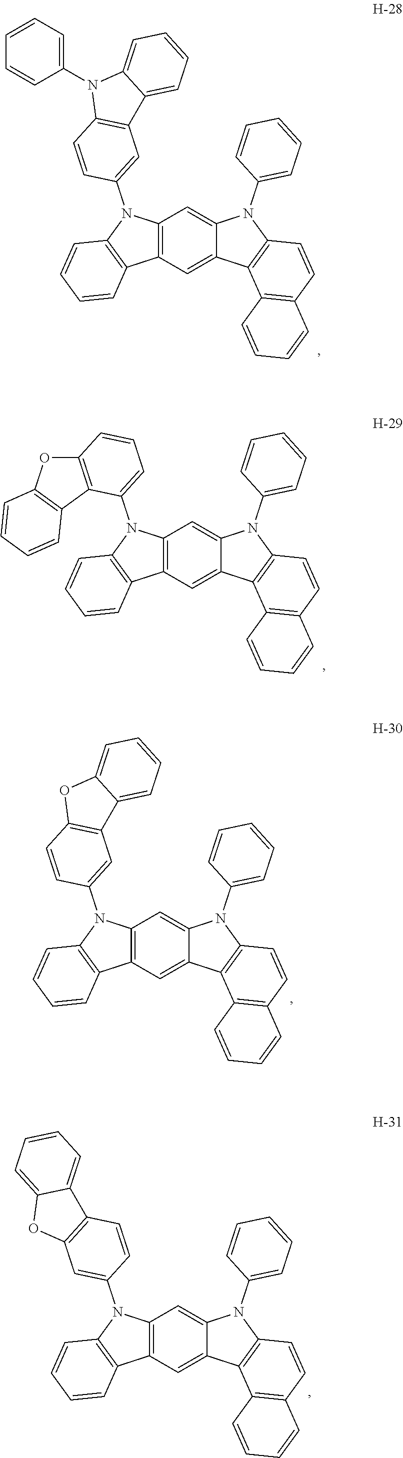 [SMILES: c1ccc(-n2c3ccccc3c3cc(-n4c5ccccc5c5cc6c7c8ccccc8ccc7n(-c7ccccc7)c6cc54)ccc32)cc1 ; c1ccc(-n2c3cc4c(cc3c3c5ccccc5ccc32)c2ccccc2n4-c2cccc3oc4ccccc4c23)cc1 ; c1ccc(-n2c3cc4c(cc3c3c5ccccc5ccc32)c2ccccc2n4-c2ccc3oc4ccccc4c3c2)cc1 ; c1ccc(-n2c3cc4c(cc3c3c5ccccc5ccc32)c2ccccc2n4-c2ccc3c(c2)oc2ccccc23)cc1]

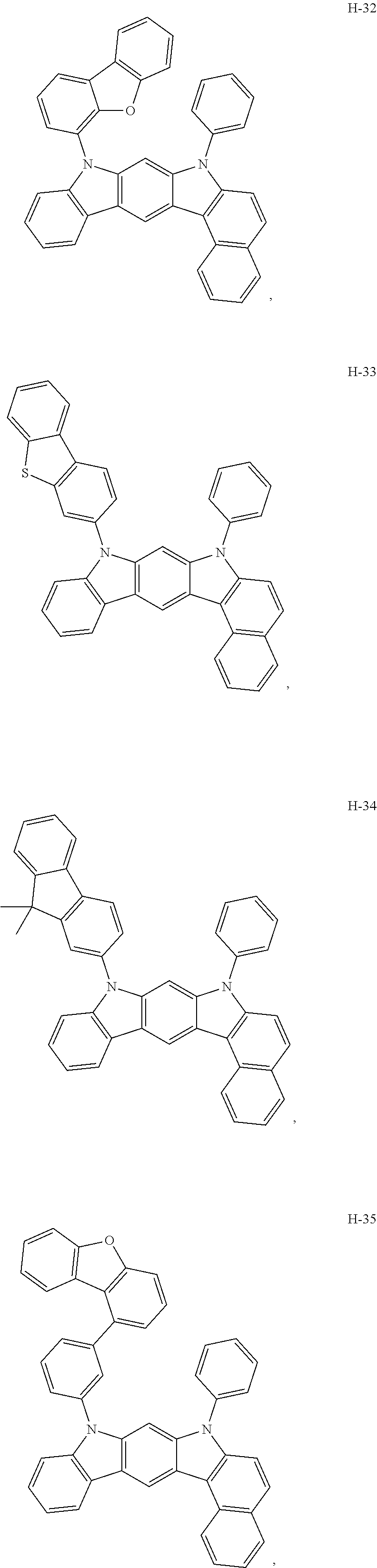 [SMILES: c1ccc(-n2c3cc4c(cc3c3c5ccccc5ccc32)c2ccccc2n4-c2cccc3c2oc2ccccc23)cc1 ; c1ccc(-n2c3cc4c(cc3c3c5ccccc5ccc32)c2ccccc2n4-c2ccc3c(c2)sc2ccccc23)cc1 ; CC1(C)c2ccccc2-c2ccc(-n3c4ccccc4c4cc5c6c7ccccc7ccc6n(-c6ccccc6)c5cc43)cc21 ; c1ccc(-n2c3cc4c(cc3c3c5ccccc5ccc32)c2ccccc2n4-c2cccc(-c3cccc4oc5ccccc5c34)c2)cc1]

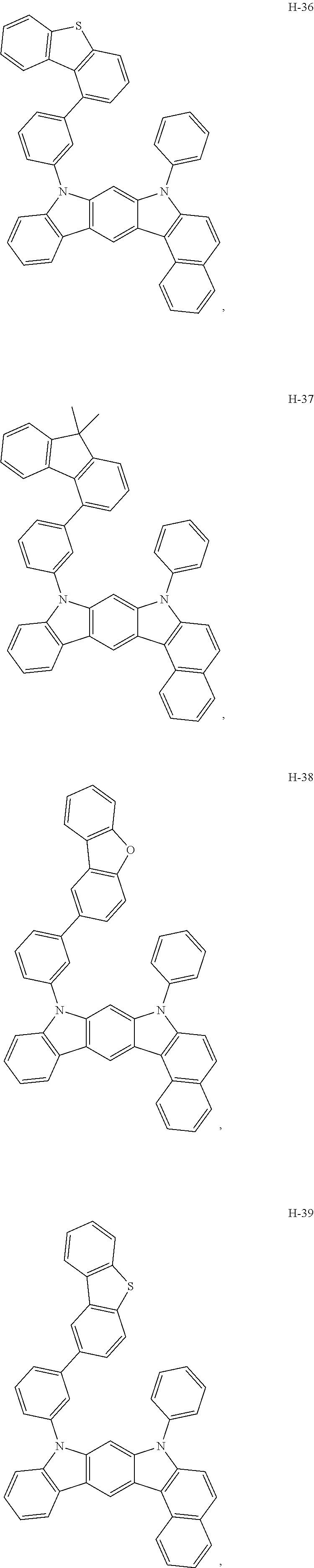 [SMILES: c1ccc(-n2c3cc4c(cc3c3c5ccccc5ccc32)c2ccccc2n4-c2cccc(-c3cccc4sc5ccccc5c34)c2)cc1 ; CC1(C)c2ccccc2-c2c(-c3cccc(-n4c5ccccc5c5cc6c7c8ccccc8ccc7n(-c7ccccc7)c6cc54)c3)cccc21 ; c1ccc(-n2c3cc4c(cc3c3c5ccccc5ccc32)c2ccccc2n4-c2cccc(-c3ccc4oc5ccccc5c4c3)c2)cc1 ; c1ccc(-n2c3cc4c(cc3c3c5ccccc5ccc32)c2ccccc2n4-c2cccc(-c3ccc4sc5ccccc5c4c3)c2)cc1]

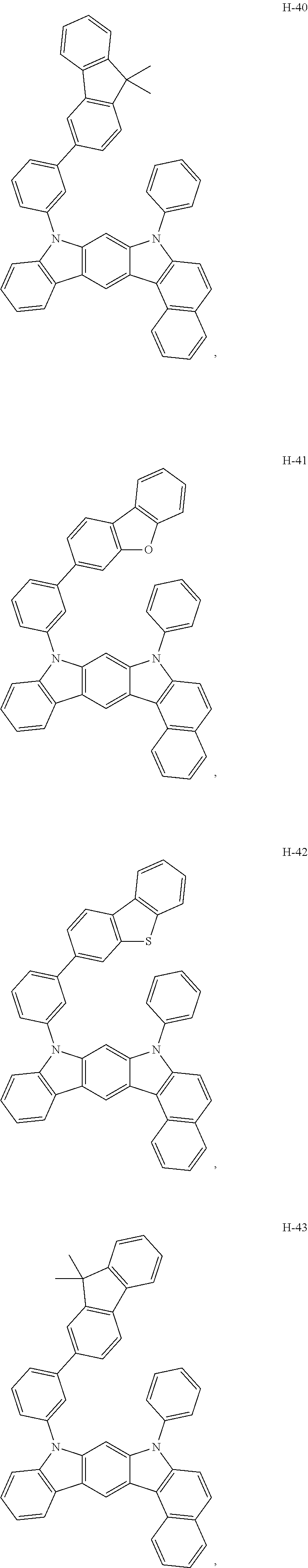 [SMILES: CC1(C)c2ccccc2-c2cc(-c3cccc(-n4c5ccccc5c5cc6c7c8ccccc8ccc7n(-c7ccccc7)c6cc54)c3)ccc21 ; c1ccc(-n2c3cc4c(cc3c3c5ccccc5ccc32)c2ccccc2n4-c2cccc(-c3ccc4c(c3)oc3ccccc34)c2)cc1 ; c1ccc(-n2c3cc4c(cc3c3c5ccccc5ccc32)c2ccccc2n4-c2cccc(-c3ccc4c(c3)sc3ccccc34)c2)cc1 ; CC1(C)c2ccccc2-c2ccc(-c3cccc(-n4c5ccccc5c5cc6c7c8ccccc8ccc7n(-c7ccccc7)c6cc54)c3)cc21]

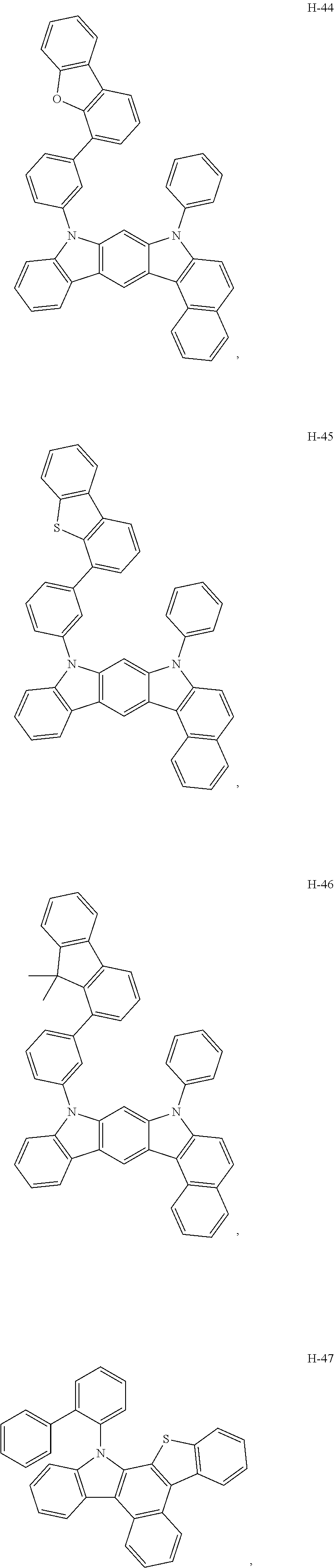 [SMILES: c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2cccc(-c3cccc4c3sc3ccccc34)c2)cc1 ; CC1(C)c2ccccc2-c2cccc(-c3cccc(-n4c5ccccc5c5cc6c7c8ccccc8ccc7n(-c7ccccc7)c6cc54)c3)c21 ; c1ccc(-c2ccccc2-n2c3ccccc3c3c4ccccc4c4c5ccccc5sc4c32)cc1]

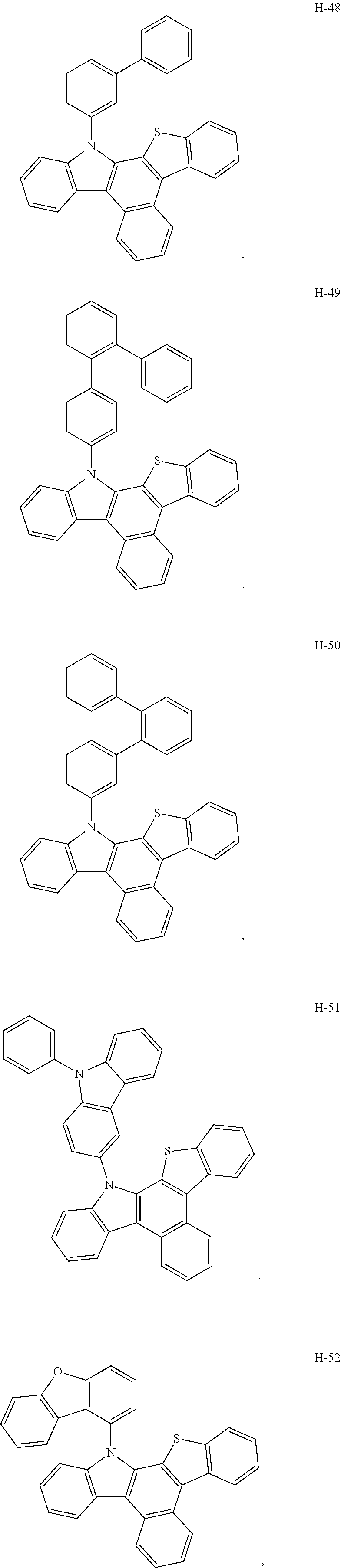 [SMILES: c1ccc(-c2cccc(-n3c4ccccc4c4c5ccccc5c5c6ccccc6sc5c43)c2)cc1 ; c1ccc(-c2ccccc2-c2ccc(-n3c4ccccc4c4c5ccccc5c5c6ccccc6sc5c43)cc2)cc1 ; c1ccc(-c2ccccc2-c2cccc(-n3c4ccccc4c4c5ccccc5c5c6ccccc6sc5c43)c2)cc1 ; c1ccc(-n2c3ccccc3c3cc(-n4c5ccccc5c5c6ccccc6c6c7ccccc7sc6c54)ccc32)cc1 ; c1ccc2c(c1)oc1cccc(-n3c4ccccc4c4c5ccccc5c5c6ccccc6sc5c43)c12]

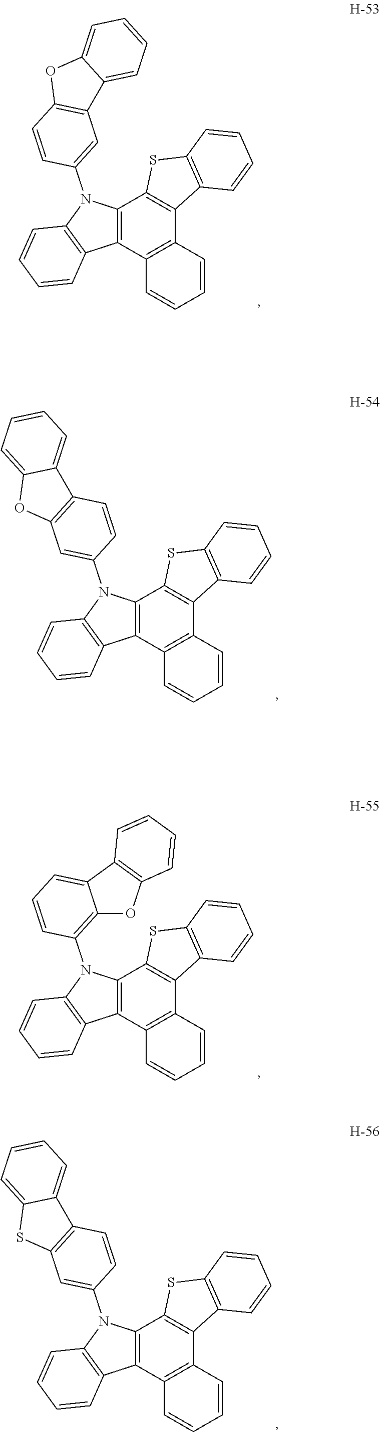 [SMILES: c1ccc2c(c1)oc1ccc(-n3c4ccccc4c4c5ccccc5c5c6ccccc6sc5c43)cc12 ; c1ccc2c(c1)oc1cc(-n3c4ccccc4c4c5ccccc5c5c6ccccc6sc5c43)ccc12 ; c1ccc2c(c1)oc1c(-n3c4ccccc4c4c5ccccc5c5c6ccccc6sc5c43)cccc12 ; c1ccc2c(c1)sc1cc(-n3c4ccccc4c4c5ccccc5c5c6ccccc6sc5c43)ccc12]

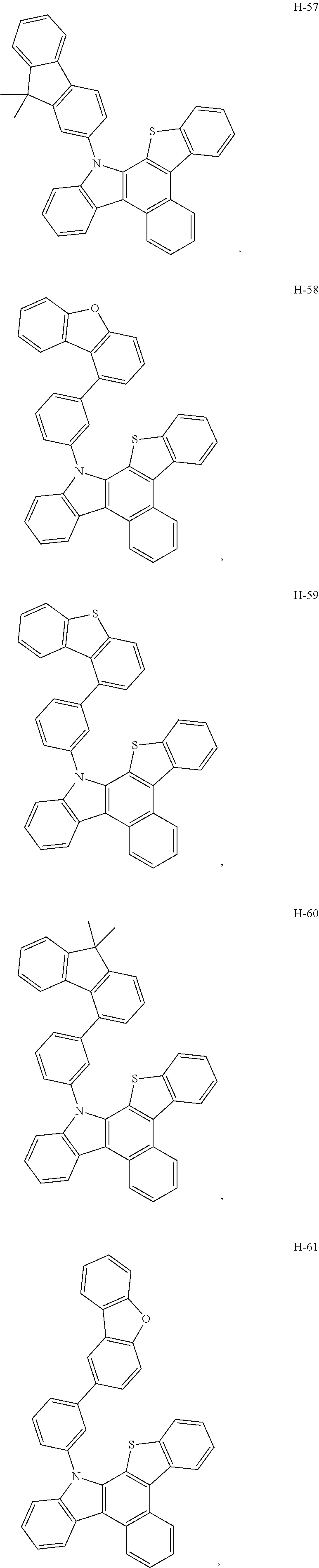 [SMILES: CC1(C)c2ccccc2-c2ccc(-n3c4ccccc4c4c5ccccc5c5c6ccccc6sc5c43)cc21 ; c1cc(-c2cccc3oc4ccccc4c23)cc(-n2c3ccccc3c3c4ccccc4c4c5ccccc5sc4c32)c1 ; c1cc(-c2cccc3sc4ccccc4c23)cc(-n2c3ccccc3c3c4ccccc4c4c5ccccc5sc4c32)c1 ; CC1(C)c2ccccc2-c2c(-c3cccc(-n4c5ccccc5c5c6ccccc6c6c7ccccc7sc6c54)c3)cccc21 ; c1cc(-c2ccc3oc4ccccc4c3c2)cc(-n2c3ccccc3c3c4ccccc4c4c5ccccc5sc4c32)c1]

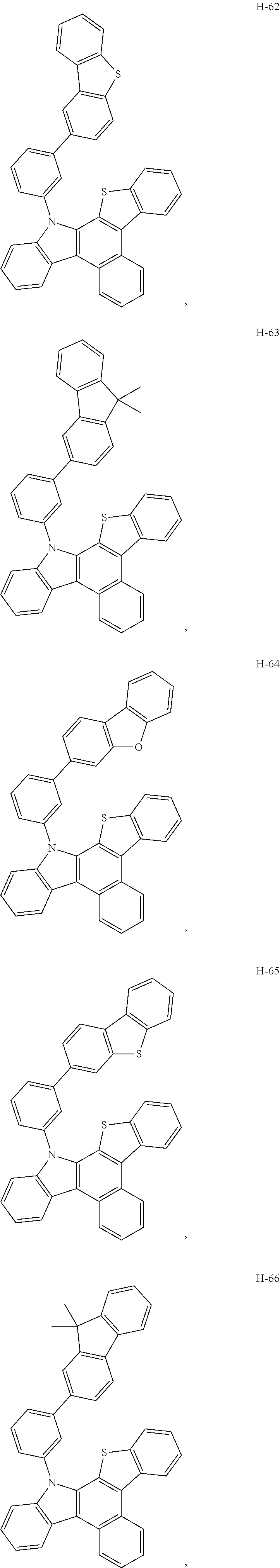 [SMILES: c1cc(-c2ccc3sc4ccccc4c3c2)cc(-n2c3ccccc3c3c4ccccc4c4c5ccccc5sc4c32)c1 ; CC1(C)c2ccccc2-c2cc(-c3cccc(-n4c5ccccc5c5c6ccccc6c6c7ccccc7sc6c54)c3)ccc21 ; c1cc(-c2ccc3c(c2)oc2ccccc23)cc(-n2c3ccccc3c3c4ccccc4c4c5ccccc5sc4c32)c1 ; c1cc(-c2ccc3c(c2)sc2ccccc23)cc(-n2c3ccccc3c3c4ccccc4c4c5ccccc5sc4c32)c1 ; CC1(C)c2ccccc2-c2ccc(-c3cccc(-n4c5ccccc5c5c6ccccc6c6c7ccccc7sc6c54)c3)cc21]

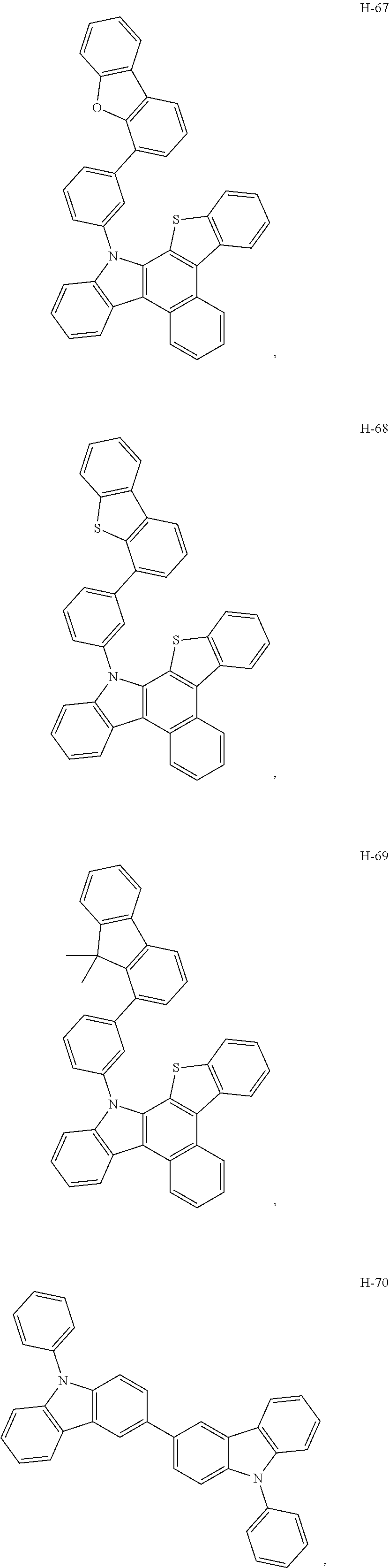 [SMILES: c1cc(-c2cccc3c2oc2ccccc23)cc(-n2c3ccccc3c3c4ccccc4c4c5ccccc5sc4c32)c1 ; c1cc(-c2cccc3c2sc2ccccc23)cc(-n2c3ccccc3c3c4ccccc4c4c5ccccc5sc4c32)c1 ; CC1(C)c2ccccc2-c2cccc(-c3cccc(-n4c5ccccc5c5c6ccccc6c6c7ccccc7sc6c54)c3)c21 ; c1ccc(-n2c3ccccc3c3cc(-c4ccc5c(c4)c4ccccc4n5-c4ccccc4)ccc32)cc1]

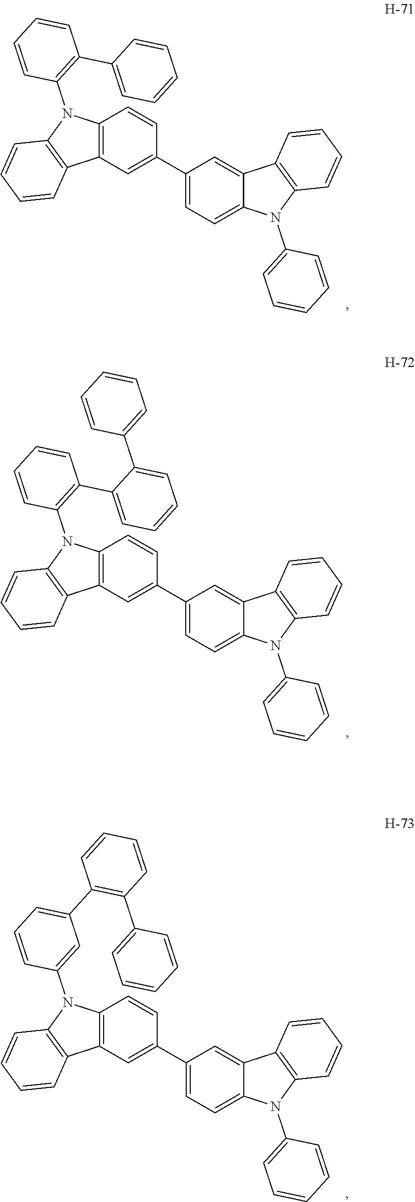 [SMILES: c1ccc(-c2ccccc2-n2c3ccccc3c3cc(-c4ccc5c(c4)c4ccccc4n5-c4ccccc4)ccc32)cc1 ; c1ccc(-c2ccccc2-c2ccccc2-n2c3ccccc3c3cc(-c4ccc5c(c4)c4ccccc4n5-c4ccccc4)ccc32)cc1 ; c1ccc(-c2ccccc2-c2cccc(-n3c4ccccc4c4cc(-c5ccc6c(c5)c5ccccc5n6-c5ccccc5)ccc43)c2)cc1]

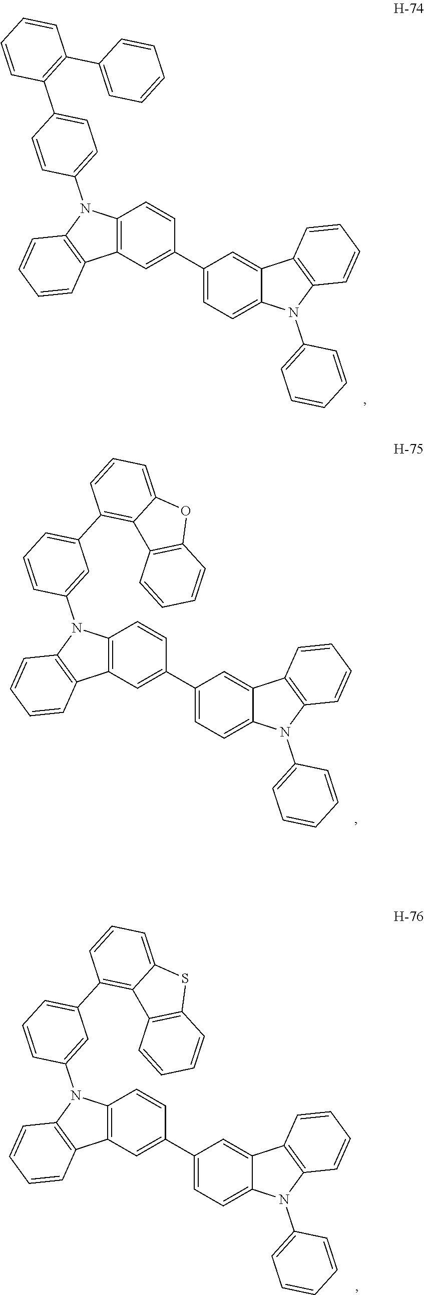 [SMILES: c1ccc(-c2ccccc2-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1]

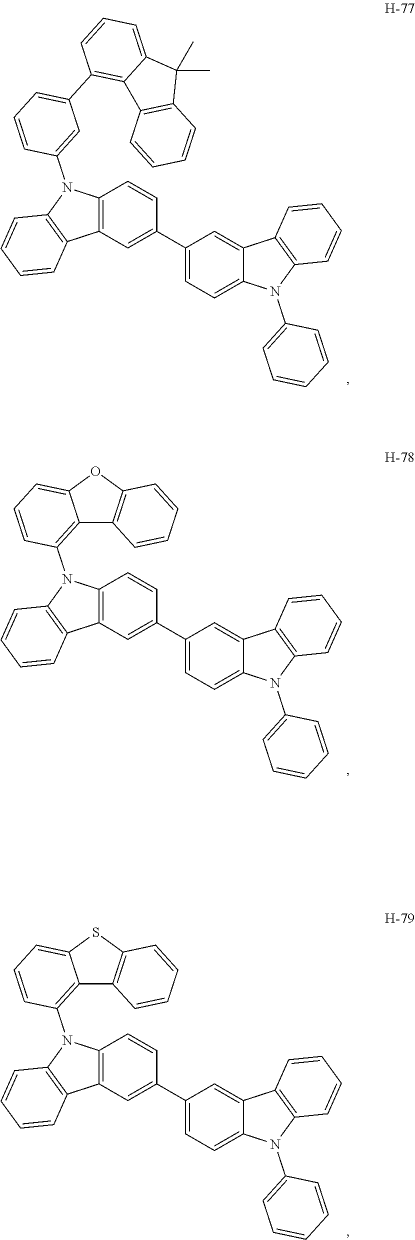 [SMILES: CC1(C)c2ccccc2-c2c(-c3cccc(-n4c5ccccc5c5cc(-c6ccc7c(c6)c6ccccc6n7-c6ccccc6)ccc54)c3)cccc21 ; c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5sc6ccccc6c45)ccc32)cc1]

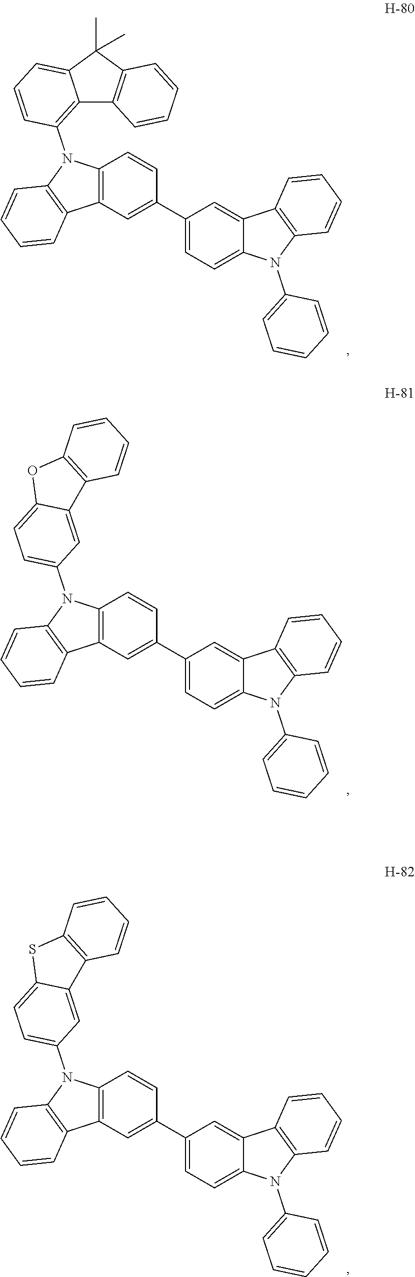 [SMILES: CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

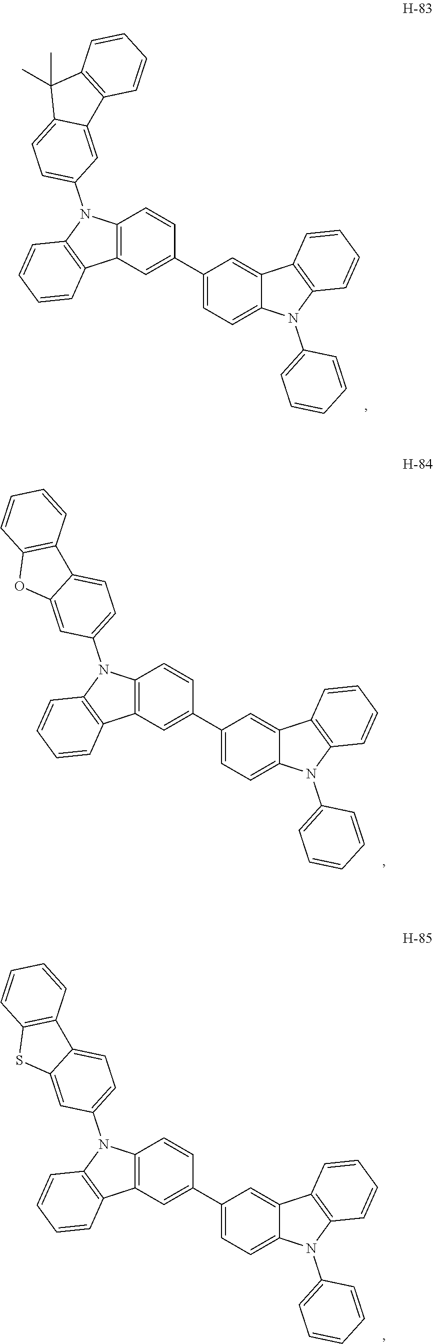 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5c(c4)sc4ccccc45)ccc32)cc1]

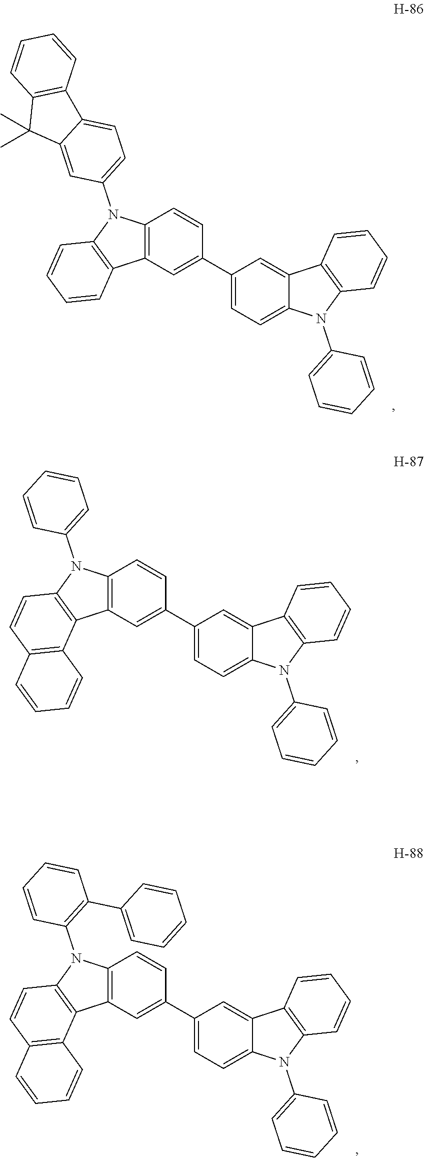 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4c6ccccc6ccc4n5-c4ccccc4)ccc32)cc1 ; c1ccc(-c2ccccc2-n2c3ccc(-c4ccc5c(c4)c4ccccc4n5-c4ccccc4)cc3c3c4ccccc4ccc32)cc1]

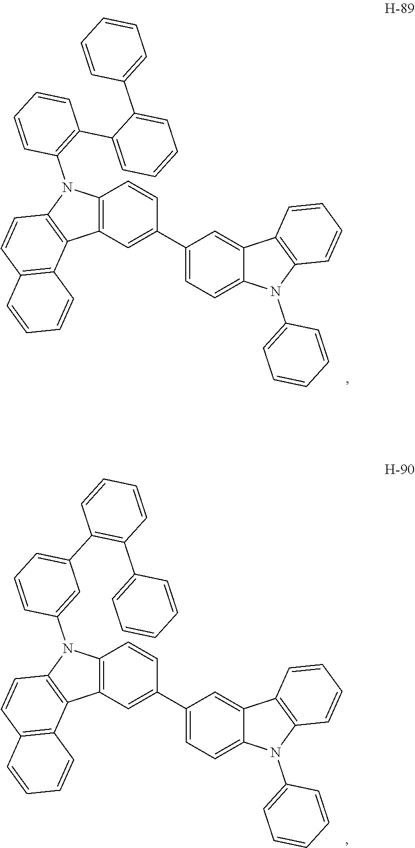 [SMILES: c1ccc(-c2ccccc2-c2ccccc2-n2c3ccc(-c4ccc5c(c4)c4ccccc4n5-c4ccccc4)cc3c3c4ccccc4ccc32)cc1 ; c1ccc(-c2ccccc2-c2cccc(-n3c4ccc(-c5ccc6c(c5)c5ccccc5n6-c5ccccc5)cc4c4c5ccccc5ccc43)c2)cc1]

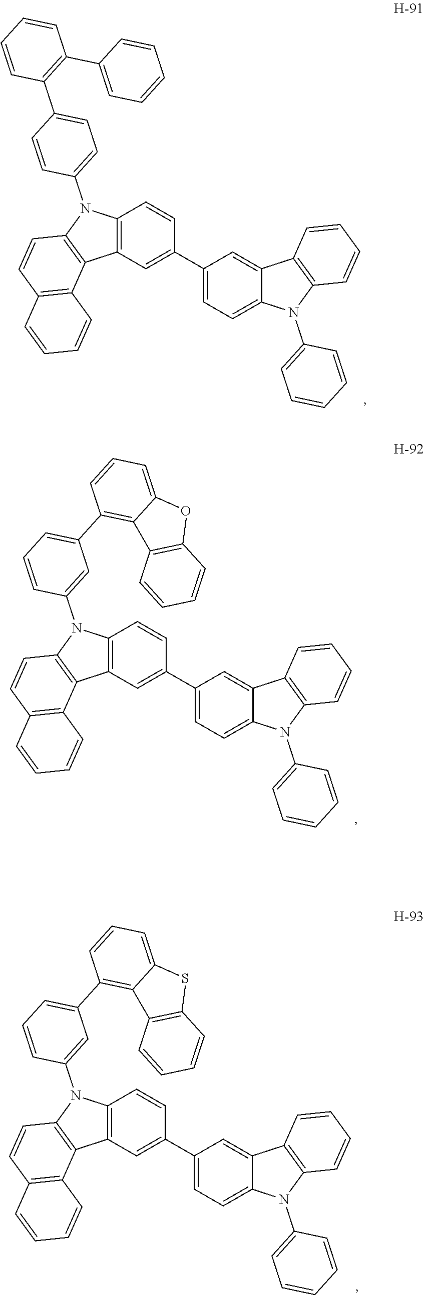 [SMILES: c1ccc(-c2ccccc2-c2ccc(-n3c4ccc(-c5ccc6c(c5)c5ccccc5n6-c5ccccc5)cc4c4c5ccccc5ccc43)cc2)cc1 ; c1ccc(-n2c3ccccc3c3cc(-c4ccc5c(c4)c4c6ccccc6ccc4n5-c4cccc(-c5cccc6oc7ccccc7c56)c4)ccc32)cc1 ; c1ccc(-n2c3ccccc3c3cc(-c4ccc5c(c4)c4c6ccccc6ccc4n5-c4cccc(-c5cccc6sc7ccccc7c56)c4)ccc32)cc1]

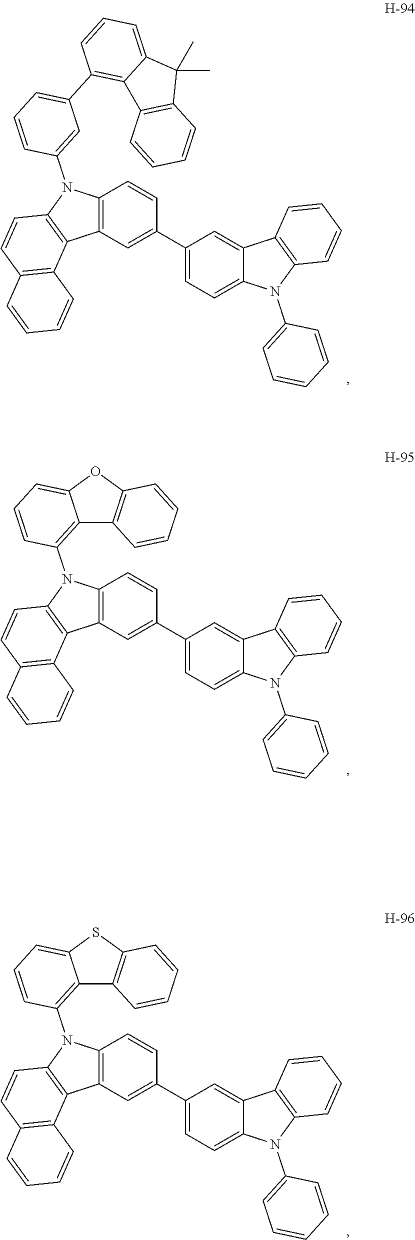 [SMILES: CC1(C)c2ccccc2-c2c(-c3cccc(-n4c5ccc(-c6ccc7c(c6)c6ccccc6n7-c6ccccc6)cc5c5c6ccccc6ccc54)c3)cccc21 ; c1ccc(-n2c3ccccc3c3cc(-c4ccc5c(c4)c4c6ccccc6ccc4n5-c4cccc5oc6ccccc6c45)ccc32)cc1 ; c1ccc(-n2c3ccccc3c3cc(-c4ccc5c(c4)c4c6ccccc6ccc4n5-c4cccc5sc6ccccc6c45)ccc32)cc1]

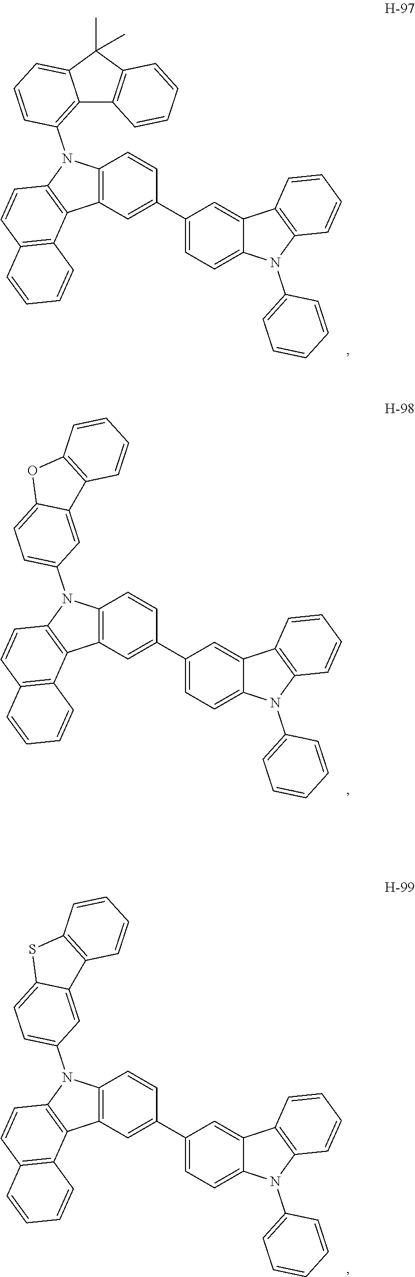 [SMILES: CC1(C)c2ccccc2-c2c(-n3c4ccc(-c5ccc6c(c5)c5ccccc5n6-c5ccccc5)cc4c4c5ccccc5ccc43)cccc21 ; c1ccc(-n2c3ccccc3c3cc(-c4ccc5c(c4)c4c6ccccc6ccc4n5-c4ccc5oc6ccccc6c5c4)ccc32)cc1 ; c1ccc(-n2c3ccccc3c3cc(-c4ccc5c(c4)c4c6ccccc6ccc4n5-c4ccc5sc6ccccc6c5c4)ccc32)cc1]

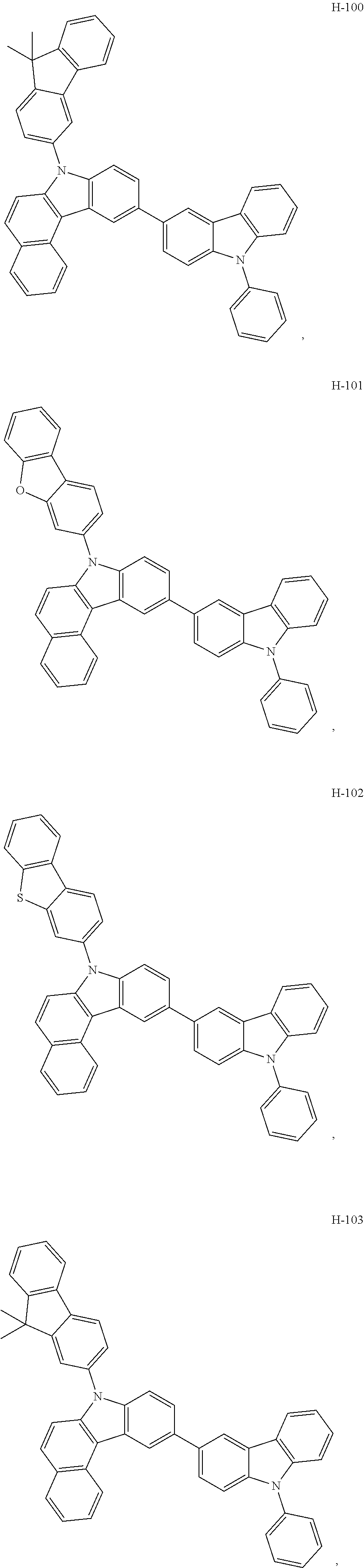 [SMILES: CC1(C)c2ccccc2-c2cc(-n3c4ccc(-c5ccc6c(c5)c5ccccc5n6-c5ccccc5)cc4c4c5ccccc5ccc43)ccc21 ; c1ccc(-n2c3ccccc3c3cc(-c4ccc5c(c4)c4c6ccccc6ccc4n5-c4ccc5c(c4)oc4ccccc45)ccc32)cc1 ; c1ccc(-n2c3ccccc3c3cc(-c4ccc5c(c4)c4c6ccccc6ccc4n5-c4ccc5c(c4)sc4ccccc45)ccc32)cc1 ; CC1(C)c2ccccc2-c2ccc(-n3c4ccc(-c5ccc6c(c5)c5ccccc5n6-c5ccccc5)cc4c4c5ccccc5ccc43)cc21]

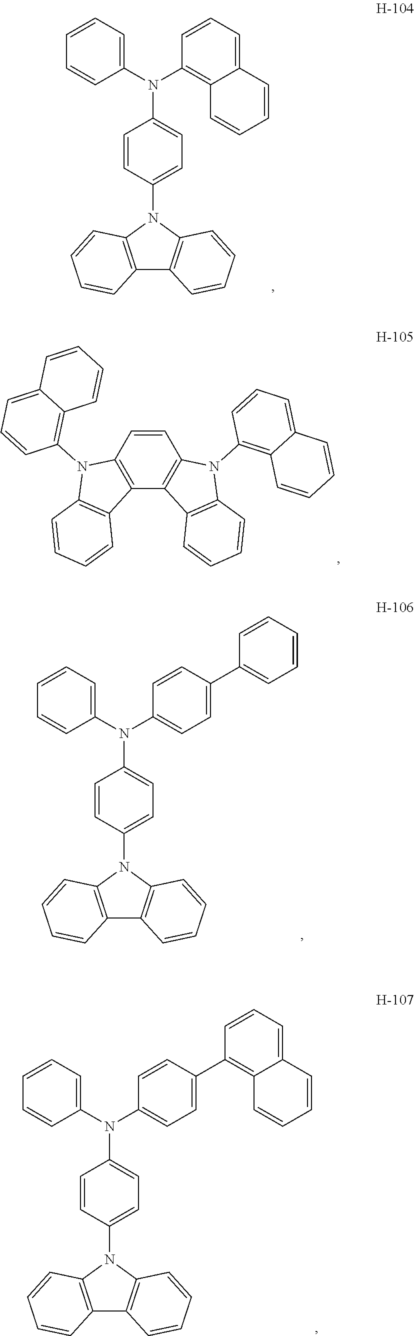 [SMILES: c1ccc(N(c2ccc(-n3c4ccccc4c4ccccc43)cc2)c2cccc3ccccc23)cc1 ; c1ccc2c(-n3c4ccccc4c4c5c6ccccc6n(-c6cccc7ccccc67)c5ccc43)cccc2c1 ; c1ccc(-c2ccc(N(c3ccccc3)c3ccc(-n4c5ccccc5c5ccccc54)cc3)cc2)cc1 ; c1ccc(N(c2ccc(-c3cccc4ccccc34)cc2)c2ccc(-n3c4ccccc4c4ccccc43)cc2)cc1]

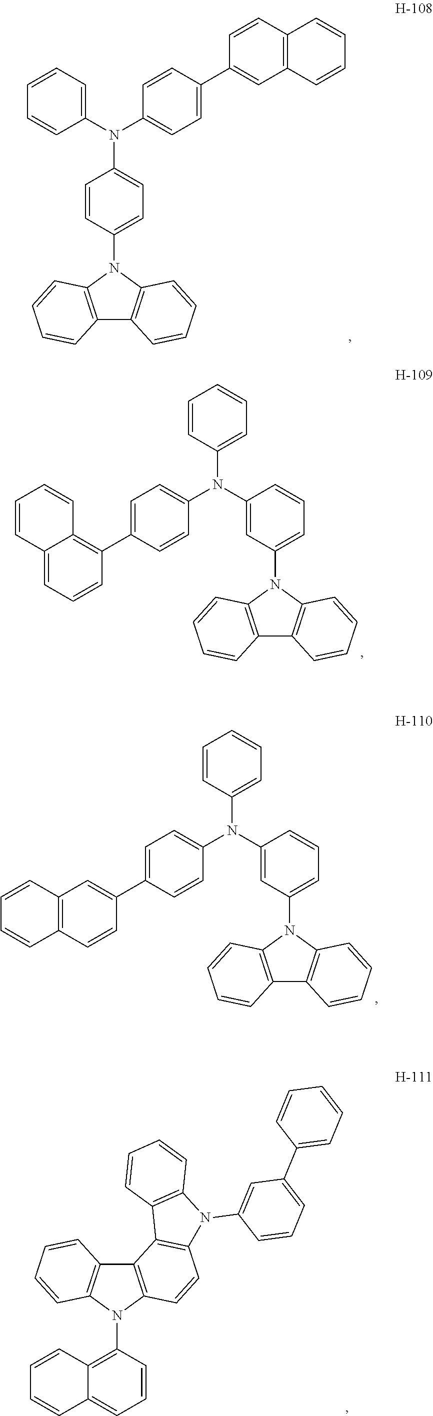 [SMILES: c1ccc(N(c2ccc(-c3ccc4ccccc4c3)cc2)c2ccc(-n3c4ccccc4c4ccccc43)cc2)cc1 ; c1ccc(N(c2ccc(-c3cccc4ccccc34)cc2)c2cccc(-n3c4ccccc4c4ccccc43)c2)cc1 ; c1ccc(N(c2ccc(-c3ccc4ccccc4c3)cc2)c2cccc(-n3c4ccccc4c4ccccc43)c2)cc1 ; c1ccc(-c2cccc(-n3c4ccccc4c4c5c6ccccc6n(-c6cccc7ccccc67)c5ccc43)c2)cc1]

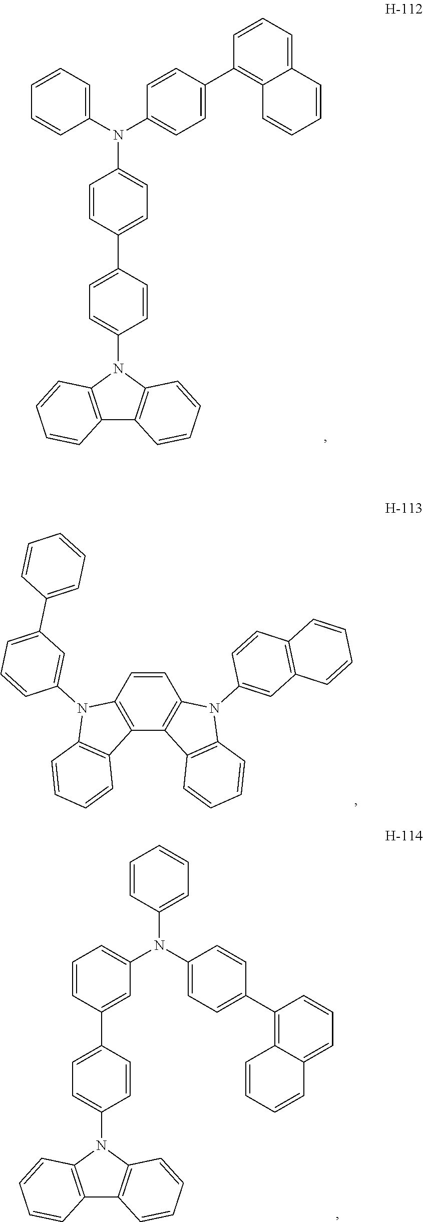 [SMILES: c1ccc(N(c2ccc(-c3ccc(-n4c5ccccc5c5ccccc54)cc3)cc2)c2ccc(-c3cccc4ccccc34)cc2)cc1 ; c1ccc(-c2cccc(-n3c4ccccc4c4c5c6ccccc6n(-c6ccc7ccccc7c6)c5ccc43)c2)cc1 ; c1ccc(N(c2ccc(-c3cccc4ccccc34)cc2)c2cccc(-c3ccc(-n4c5ccccc5c5ccccc54)cc3)c2)cc1]

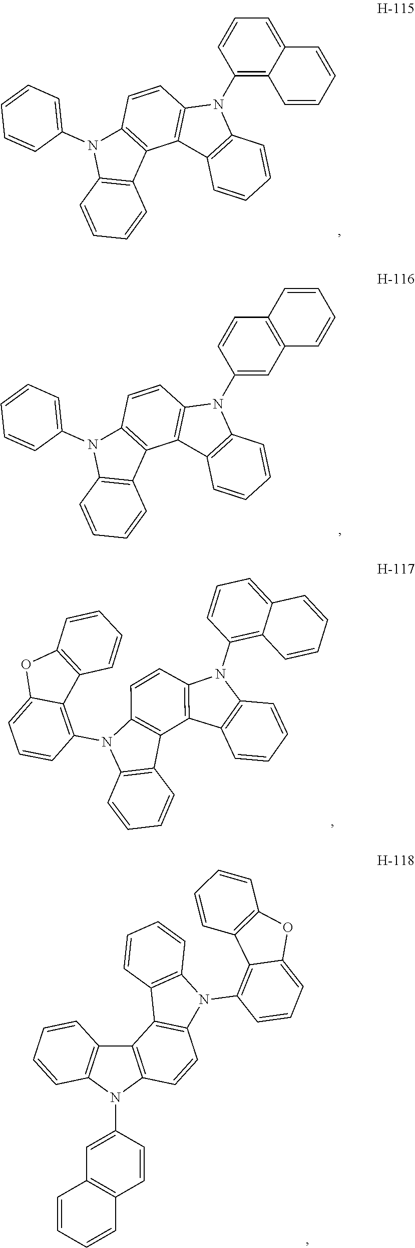 [SMILES: c1ccc(-n2c3ccccc3c3c4c5ccccc5n(-c5cccc6ccccc56)c4ccc32)cc1 ; c1ccc(-n2c3ccccc3c3c4c5ccccc5n(-c5ccc6ccccc6c5)c4ccc32)cc1 ; c1ccc2c(-n3c4ccccc4c4c5c6ccccc6n(-c6cccc7oc8ccccc8c67)c5ccc43)cccc2c1 ; c1ccc2cc(-n3c4ccccc4c4c5c6ccccc6n(-c6cccc7oc8ccccc8c67)c5ccc43)ccc2c1]

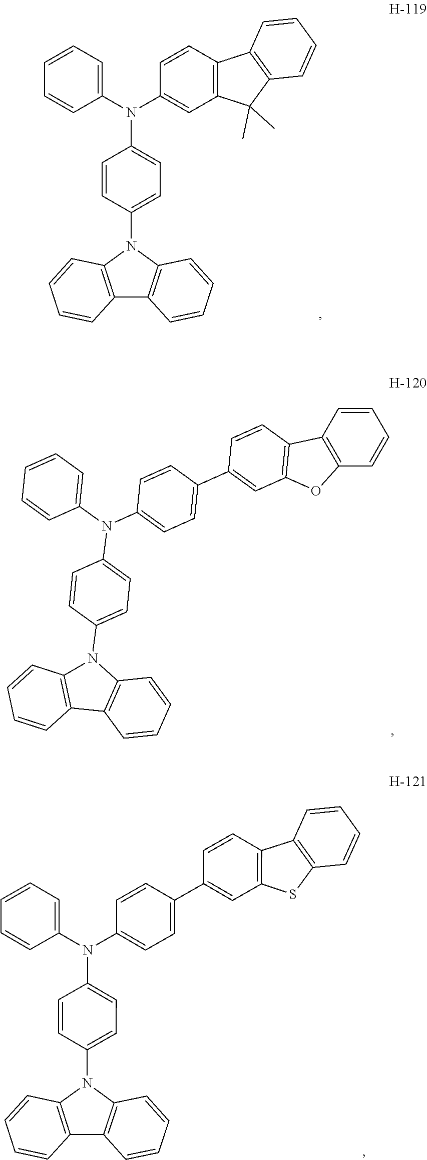 [SMILES: CC1(C)c2ccccc2-c2ccc(N(c3ccccc3)c3ccc(-n4c5ccccc5c5ccccc54)cc3)cc21 ; c1ccc(N(c2ccc(-c3ccc4c(c3)oc3ccccc34)cc2)c2ccc(-n3c4ccccc4c4ccccc43)cc2)cc1 ; c1ccc(N(c2ccc(-c3ccc4c(c3)sc3ccccc34)cc2)c2ccc(-n3c4ccccc4c4ccccc43)cc2)cc1]

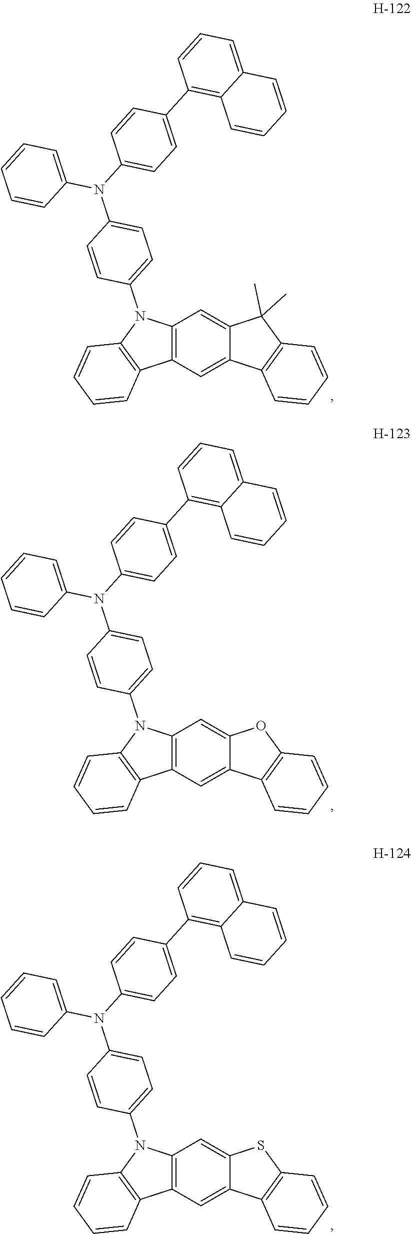 [SMILES: CC1(C)c2ccccc2-c2cc3c4ccccc4n(-c4ccc(N(c5ccccc5)c5ccc(-c6cccc7ccccc67)cc5)cc4)c3cc21 ; c1ccc(N(c2ccc(-c3cccc4ccccc34)cc2)c2ccc(-n3c4ccccc4c4cc5c(cc43)oc3ccccc35)cc2)cc1 ; c1ccc(N(c2ccc(-c3cccc4ccccc34)cc2)c2ccc(-n3c4ccccc4c4cc5c(cc43)sc3ccccc35)cc2)cc1]

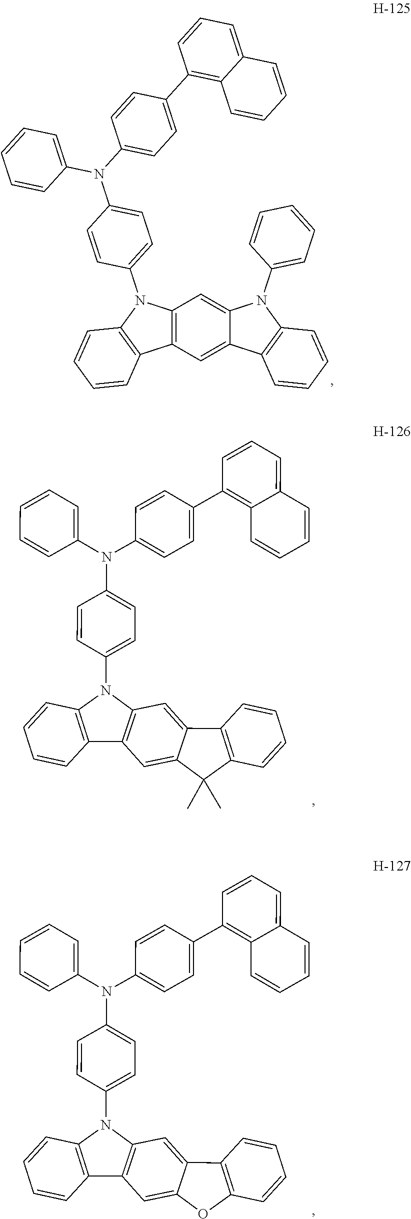 [SMILES: c1ccc(N(c2ccc(-c3cccc4ccccc34)cc2)c2ccc(-n3c4ccccc4c4cc5c6ccccc6n(-c6ccccc6)c5cc43)cc2)cc1 ; CC1(C)c2ccccc2-c2cc3c(cc21)c1ccccc1n3-c1ccc(N(c2ccccc2)c2ccc(-c3cccc4ccccc34)cc2)cc1 ; c1ccc(N(c2ccc(-c3cccc4ccccc34)cc2)c2ccc(-n3c4ccccc4c4cc5oc6ccccc6c5cc43)cc2)cc1]

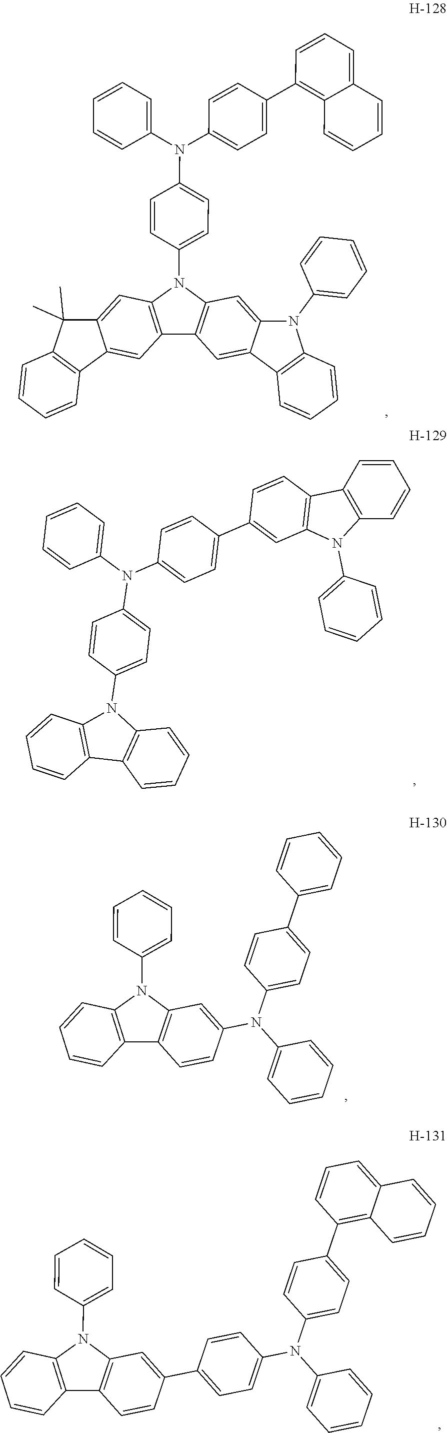 [SMILES: CC1(C)c2ccccc2-c2cc3c4cc5c6ccccc6n(-c6ccccc6)c5cc4n(-c4ccc(N(c5ccccc5)c5ccc(-c6cccc7ccccc67)cc5)cc4)c3cc21 ; c1ccc(N(c2ccc(-c3ccc4c5ccccc5n(-c5ccccc5)c4c3)cc2)c2ccc(-n3c4ccccc4c4ccccc43)cc2)cc1 ; c1ccc(-c2ccc(N(c3ccccc3)c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cccc4ccccc34)cc2)cc1]

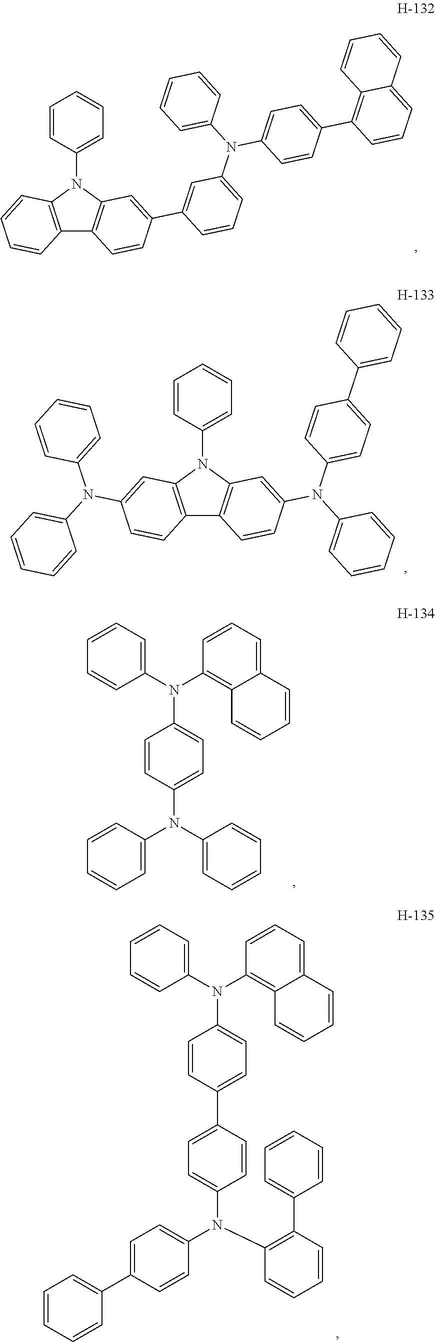 [SMILES: c1ccc(N(c2ccc(-c3cccc4ccccc34)cc2)c2cccc(-c3ccc4c5ccccc5n(-c5ccccc5)c4c3)c2)cc1 ; c1ccc(-c2ccc(N(c3ccccc3)c3ccc4c5ccc(N(c6ccccc6)c6ccccc6)cc5n(-c5ccccc5)c4c3)cc2)cc1 ; c1ccc(N(c2ccccc2)c2ccc(N(c3ccccc3)c3cccc4ccccc34)cc2)cc1 ; c1ccc(-c2ccc(N(c3ccc(-c4ccc(N(c5ccccc5)c5cccc6ccccc56)cc4)cc3)c3ccccc3-c3ccccc3)cc2)cc1]

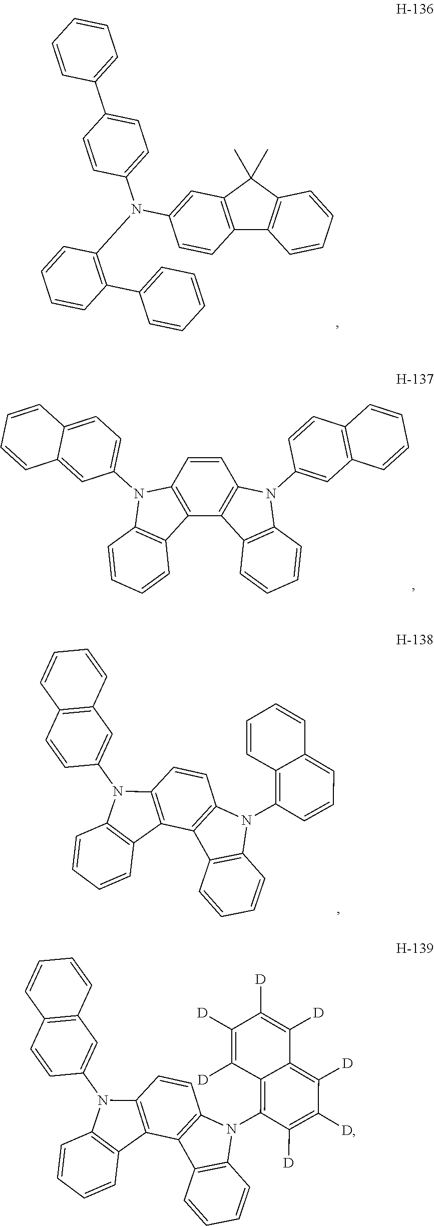

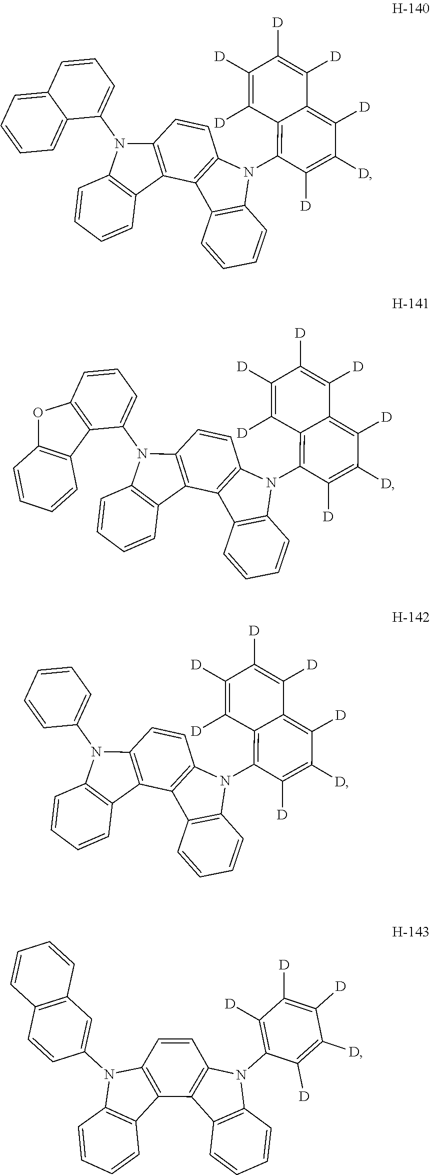

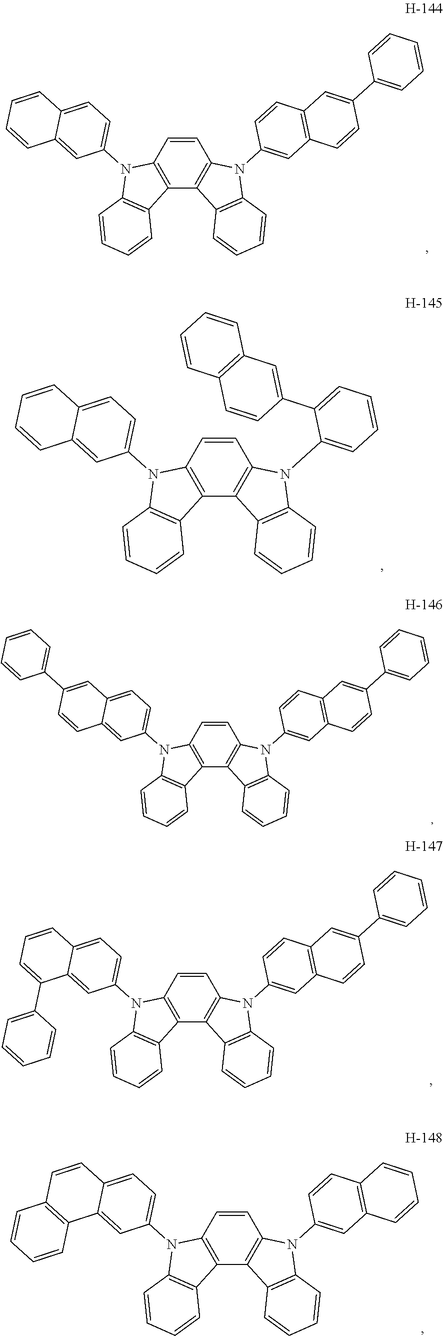 [SMILES: c1ccc(-c2ccc3cc(-n4c5ccccc5c5c6c7ccccc7n(-c7ccc8ccccc8c7)c6ccc54)ccc3c2)cc1 ; c1ccc(-n2c3ccccc3c3c4c5ccccc5n(-c5ccc6ccccc6c5)c4ccc32)c(-c2ccc3ccccc3c2)c1 ; c1ccc(-c2ccc3cc(-n4c5ccccc5c5c6c7ccccc7n(-c7ccc8cc(-c9ccccc9)ccc8c7)c6ccc54)ccc3c2)cc1 ; c1ccc(-c2ccc3cc(-n4c5ccccc5c5c6c7ccccc7n(-c7ccc8cccc(-c9ccccc9)c8c7)c6ccc54)ccc3c2)cc1 ; c1ccc2cc(-n3c4ccccc4c4c5c6ccccc6n(-c6ccc7ccc8ccccc8c7c6)c5ccc43)ccc2c1]

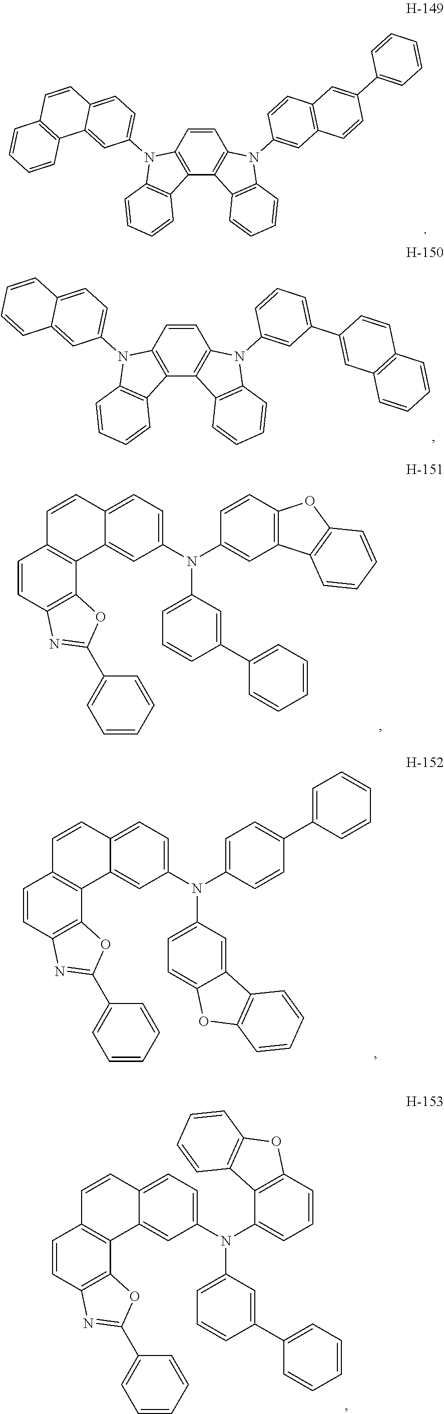 [SMILES: c1ccc(-c2ccc3cc(-n4c5ccccc5c5c6c7ccccc7n(-c7ccc8ccc9ccccc9c8c7)c6ccc54)ccc3c2)cc1 ; c1cc(-c2ccc3ccccc3c2)cc(-n2c3ccccc3c3c4c5ccccc5n(-c5ccc6ccccc6c5)c4ccc32)c1 ; c1ccc(-c2cccc(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2cccc(N(c3ccc4ccc5ccc6nc(-c7ccccc7)oc6c5c4c3)c3cccc4oc5ccccc5c34)c2)cc1]

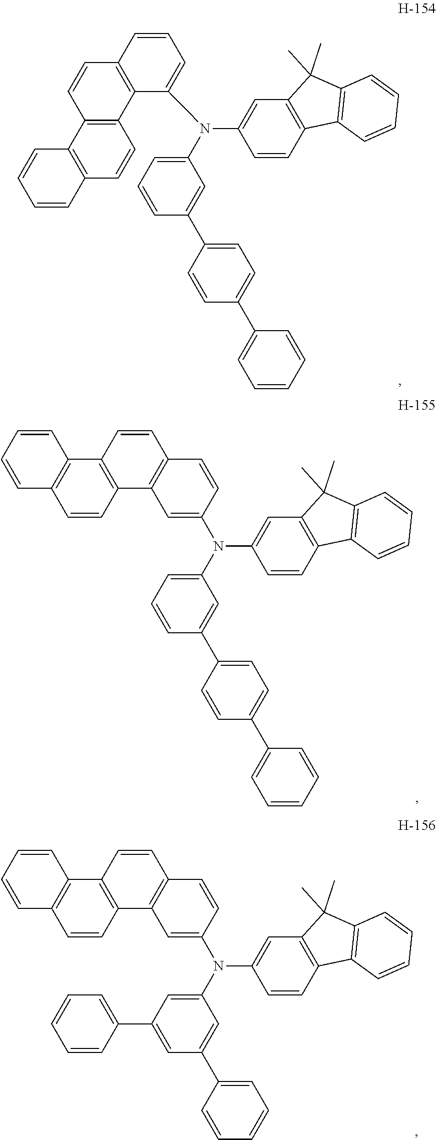 [SMILES: CC1(C)c2ccccc2-c2ccc(N(c3cccc(-c4ccc(-c5ccccc5)cc4)c3)c3cccc4ccc5c6ccccc6ccc5c34)cc21 ; CC1(C)c2ccccc2-c2ccc(N(c3cccc(-c4ccc(-c5ccccc5)cc4)c3)c3ccc4ccc5c6ccccc6ccc5c4c3)cc21 ; CC1(C)c2ccccc2-c2ccc(N(c3cc(-c4ccccc4)cc(-c4ccccc4)c3)c3ccc4ccc5c6ccccc6ccc5c4c3)cc21]

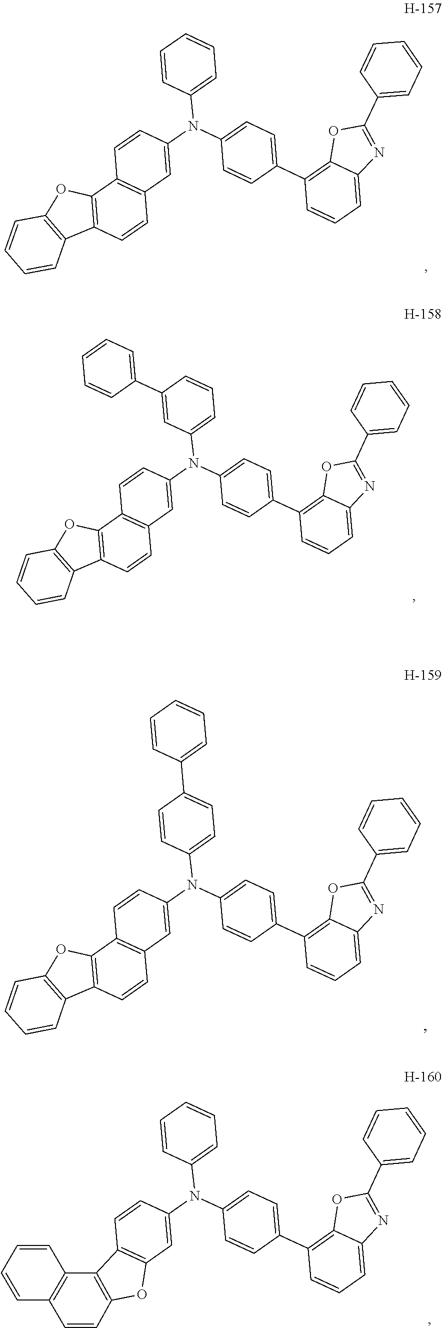 [SMILES: c1ccc(-c2nc3cccc(-c4ccc(N(c5ccccc5)c5ccc6c(ccc7c8ccccc8oc67)c5)cc4)c3o2)cc1 ; c1ccc(-c2cccc(N(c3ccc(-c4cccc5nc(-c6ccccc6)oc45)cc3)c3ccc4c(ccc5c6ccccc6oc45)c3)c2)cc1 ; c1ccc(-c2ccc(N(c3ccc(-c4cccc5nc(-c6ccccc6)oc45)cc3)c3ccc4c(ccc5c6ccccc6oc45)c3)cc2)cc1 ; c1ccc(-c2nc3cccc(-c4ccc(N(c5ccccc5)c5ccc6c(c5)oc5ccc7ccccc7c56)cc4)c3o2)cc1]

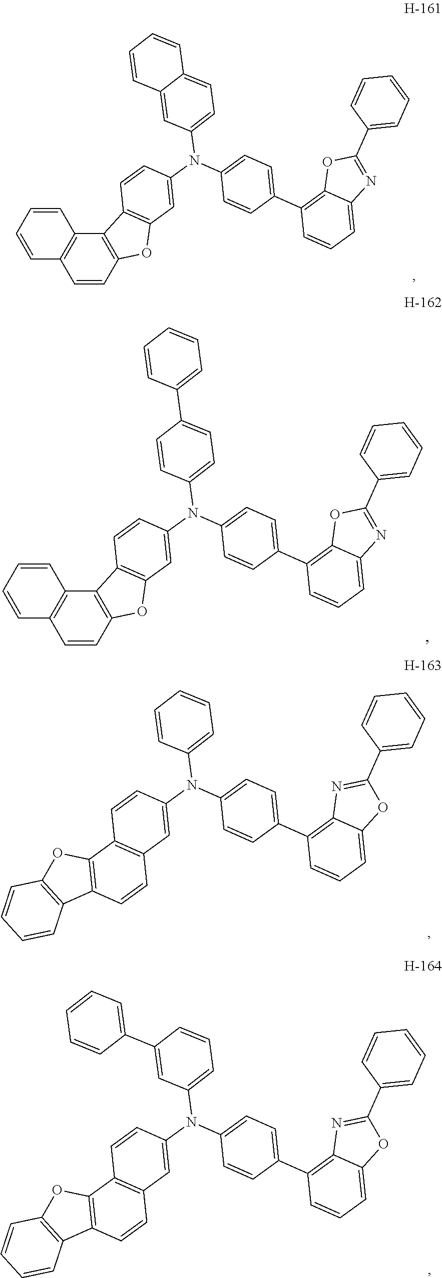 [SMILES: c1ccc(-c2nc3cccc(-c4ccc(N(c5ccc6ccccc6c5)c5ccc6c(c5)oc5ccc7ccccc7c56)cc4)c3o2)cc1 ; c1ccc(-c2ccc(N(c3ccc(-c4cccc5nc(-c6ccccc6)oc45)cc3)c3ccc4c(c3)oc3ccc5ccccc5c34)cc2)cc1 ; c1ccc(-c2nc3c(-c4ccc(N(c5ccccc5)c5ccc6c(ccc7c8ccccc8oc67)c5)cc4)cccc3o2)cc1 ; c1ccc(-c2cccc(N(c3ccc(-c4cccc5oc(-c6ccccc6)nc45)cc3)c3ccc4c(ccc5c6ccccc6oc45)c3)c2)cc1]

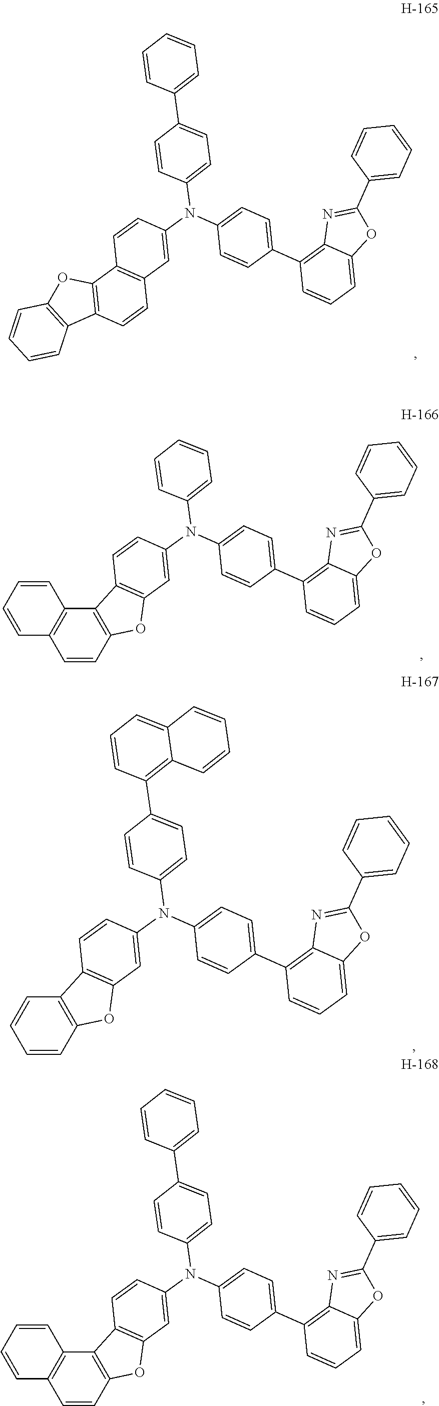 [SMILES: c1ccc(-c2ccc(N(c3ccc(-c4cccc5oc(-c6ccccc6)nc45)cc3)c3ccc4c(ccc5c6ccccc6oc45)c3)cc2)cc1 ; c1ccc(-c2nc3c(-c4ccc(N(c5ccccc5)c5ccc6c(c5)oc5ccc7ccccc7c56)cc4)cccc3o2)cc1 ; c1ccc(-c2nc3c(-c4ccc(N(c5ccc(-c6cccc7ccccc67)cc5)c5ccc6c(c5)oc5ccccc56)cc4)cccc3o2)cc1 ; c1ccc(-c2ccc(N(c3ccc(-c4cccc5oc(-c6ccccc6)nc45)cc3)c3ccc4c(c3)oc3ccc5ccccc5c34)cc2)cc1]

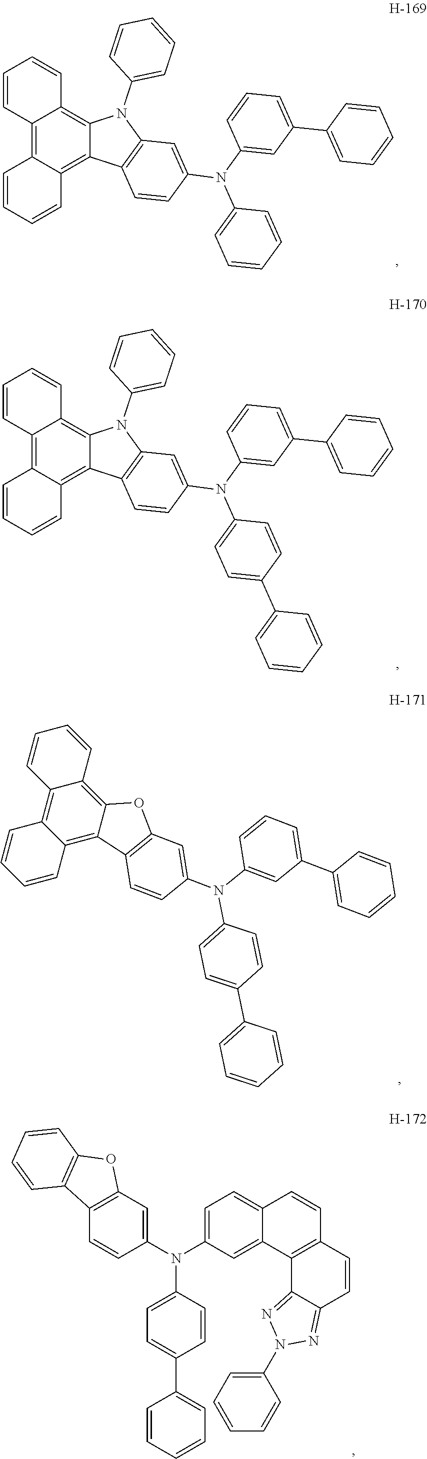 [SMILES: c1ccc(-c2cccc(N(c3ccccc3)c3ccc4c5c6ccccc6c6ccccc6c5n(-c5ccccc5)c4c3)c2)cc1 ; c1ccc(-c2ccc(N(c3cccc(-c4ccccc4)c3)c3ccc4c5c6ccccc6c6ccccc6c5n(-c5ccccc5)c4c3)cc2)cc1 ; c1ccc(-c2ccc(N(c3cccc(-c4ccccc4)c3)c3ccc4c(c3)oc3c5ccccc5c5ccccc5c43)cc2)cc1 ; c1ccc(-c2ccc(N(c3ccc4c(c3)oc3ccccc34)c3ccc4ccc5ccc6nn(-c7ccccc7)nc6c5c4c3)cc2)cc1]

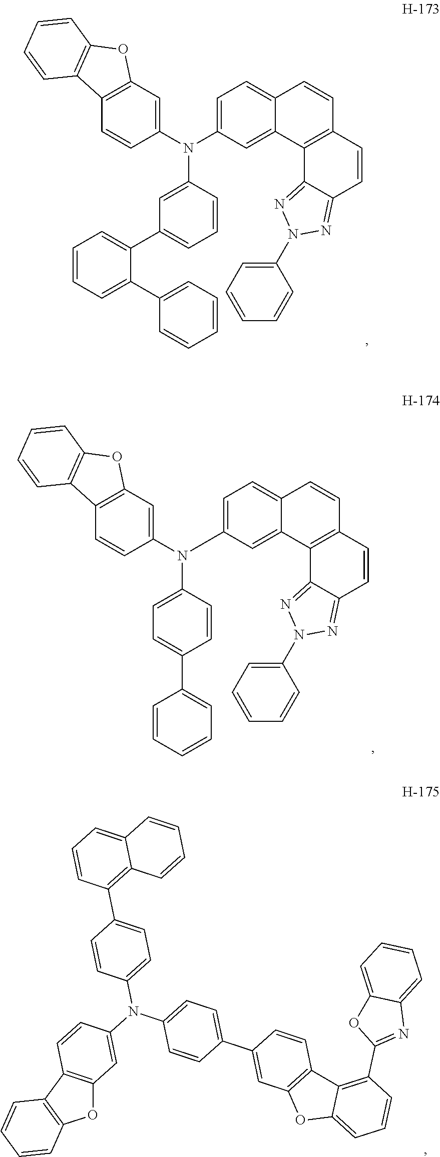 [SMILES: c1ccc(-c2ccccc2-c2cccc(N(c3ccc4c(c3)oc3ccccc34)c3ccc4ccc5ccc6nn(-c7ccccc7)nc6c5c4c3)c2)cc1 ; c1ccc(-c2ccc(N(c3ccc4c(c3)oc3ccccc34)c3ccc4ccc5ccc6nn(-c7ccccc7)nc6c5c4c3)cc2)cc1 ; c1ccc2c(-c3ccc(N(c4ccc(-c5ccc6c(c5)oc5cccc(-c7nc8ccccc8o7)c56)cc4)c4ccc5c(c4)oc4ccccc45)cc3)cccc2c1]

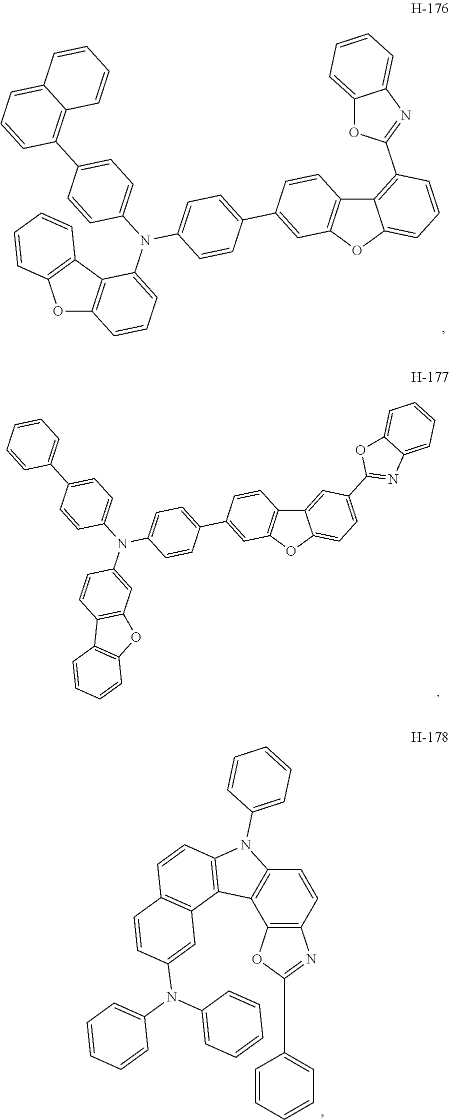 [SMILES: c1ccc2c(-c3ccc(N(c4ccc(-c5ccc6c(c5)oc5cccc(-c7nc8ccccc8o7)c56)cc4)c4cccc5oc6ccccc6c45)cc3)cccc2c1 ; c1ccc(-c2ccc(N(c3ccc(-c4ccc5c(c4)oc4ccc(-c6nc7ccccc7o6)cc45)cc3)c3ccc4c(c3)oc3ccccc34)cc2)cc1 ; c1ccc(-c2nc3ccc4c(c3o2)c2c3cc(N(c5ccccc5)c5ccccc5)ccc3ccc2n4-c2ccccc2)cc1]

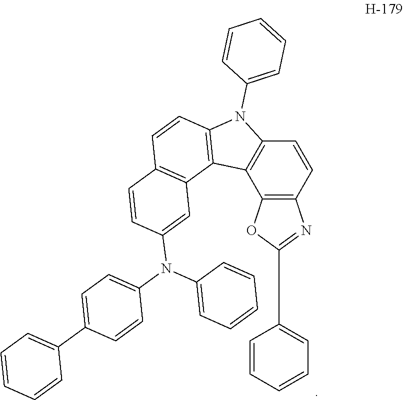 [SMILES: c1ccc(-c2ccc(N(c3ccccc3)c3ccc4ccc5c(c4c3)c3c4oc(-c6ccccc6)nc4ccc3n5-c3ccccc3)cc2)cc1]

13. The organic electroluminescent device of claim 11, wherein the organic layer is the emissive layer, and the emissive layer comprises at least one phosphorescent material.

14. The organic electroluminescent device of claim 13, wherein the phosphorescent material is a metal complex having a general formula of M(Lₐ)ₘ(L_{b})ₙ(L_{c})_{q};
M is selected from a metal with a relative atomic mass greater than 40;
Lₐ, L_{b}, and L_{c} are a first ligand, a second ligand, and a third ligand coordinated to M, respectively;
Lₐ, L_{b}, and L_{c} can be optionally joined to form a multidentate ligand;
Lₐ, L_{b}, and L_{c} may be identical or different; m is 1, 2, or 3, n is 0, 1, or 2, q is 0, 1, or 2, and m+n+q is equal to the oxidation state of M; when m is greater than or equal to 2, multiple Lₐ may be identical or different; when n is 2, two L_{b} may be identical or different; when q is 2, two L_{c} may be identical or different;
Lₐ is, at each occurrence identically or differently, selected from a structure represented by Formula 3: 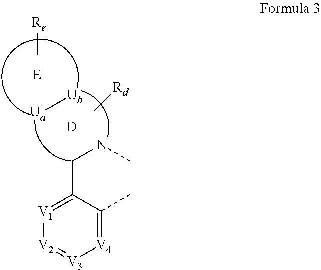
wherein
the ring D is selected from a five-membered heteroaromatic ring or a six-membered heteroaromatic ring;
the ring E is selected from a five-membered unsaturated carbocyclic ring, a benzene ring, a five-membered heteroaromatic ring, or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions, or non-substitution;
V₁ to V₄ are, at each occurrence identically or differently, selected from CRᵥ or N;
R_{d}, Rₑ, and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ, and Rᵥ can be optionally joined to form a ring;
L_{b} and L_{c} are, at each occurrence identically or differently, selected from any one of the following structures: 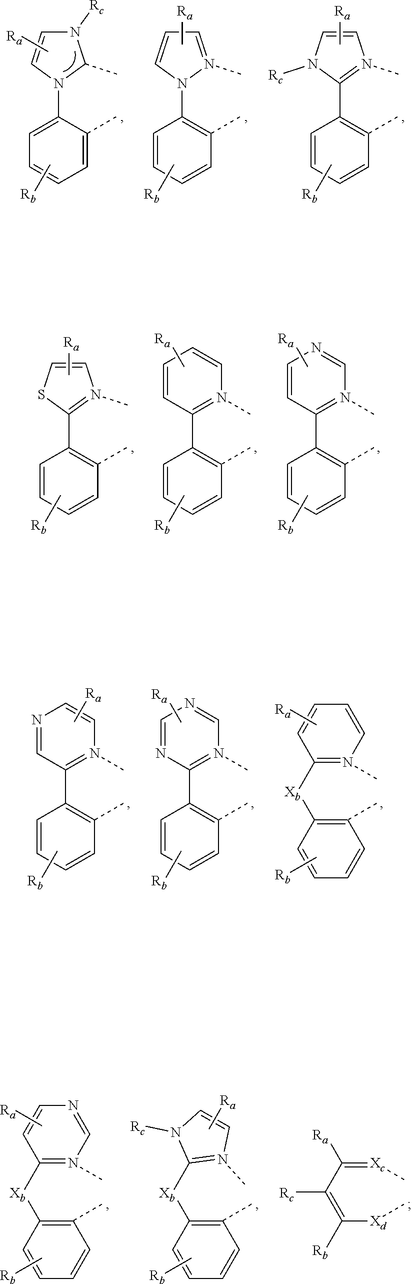
wherein
Rₐ, R_{b}, and R_{c} represent, at each occurrence identically or differently, mono-substitution, multiple substitutions, or non-substitution;
X_{b} is, at each occurrence identically or differently, selected from the group consisting of: 0, S, Se, NR_{N1}, and CR_{C1}R_{C2};
X_{c} and X_{d} are, at each occurrence identically or differently, selected from the group consisting of: O, S, Se, and NR_{N2};
Rₐ, R_{b}, Rₑ, R_{N1}, R_{N2}, R_{C1}, and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₐ, R_{b}, R_{c}, R_{N1}, R_{N2}, R_{C1}, and R_{C2} can be optionally joined to form a ring.

15. A compound composition, comprising the compound of claim 1.

16. The compound composition of claim 15, comprising a second compound having a structure represented by any one of Formula 2-1 to Formula 2-3: 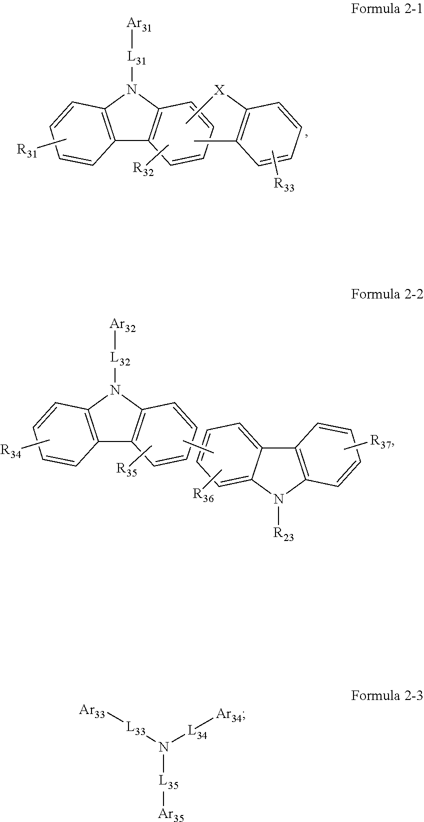
wherein
in Formula 2-1 to Formula 2-3, Ar₃₁ to Ar₃₅ are, at each occurrence identically or differently, selected from substituted or unsubstituted aryl having 6 to 30 carbon atoms or substituted or unsubstituted heteroaryl having 3 to 30 carbon atoms;
L₃₁ to L₃₅ are, at each occurrence identically or differently, selected from a single bond, substituted or unsubstituted arylene having 6 to 30 carbon atoms, substituted or unsubstituted heteroarylene having 3 to 30 carbon atoms, or a combination thereof;
X is, at each occurrence identically or differently, selected from O, S, CR₂₁R₂₂, or NR₂₃;
R₃₁ to R₃₇ represent, at each occurrence identically or differently, mono-substitution, multiple substitutions, or non-substitution;
R₃₁ to R₃₇ and R₂₁ to R₂₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₃₁ to R₃₇ can be optionally joined to form a ring.

17. An electronic apparatus, comprising the organic electroluminescent device of claim 10.
